(19) United States
(12) Patent Application Publication
(10) Pub. No.: US 2023/0365990 A1
(43) Pub. Date: Nov. 16, 2023

(54) NUCLEIC ACID CONSTRUCT FOR INCREASING ADENO-ASSOCIATED VIRUS YIELD, AND CONSTRUCTION METHOD THEREFOR

(71) Applicant: HANGZHOU GENEWAY BIOTECHNOLOGY CO., LTD., HAngzhou, Zhejiang (CN)

(72) Inventors: Yu TAO, Hangzhou, Zhejiang (CN); Haoquan WU, Hangzhou, Zhejiang (CN); Lingling SU, Hangzhou, Zhejiang (CN); Ying DANG, Hangzhou, Zhejiang (CN)

(73) Assignee: HANGZHOU GENEWAY BIOTECHNOLOGY CO., LTD., Hangzhou, Zhejiang (CN)

(21) Appl. No.: 18/022,001

(22) PCT Filed: Sep. 28, 2021
(86) PCT No.: PCT/CN2021/121350

§ 371 Date: Mar. 13, 2023

(30) Foreign Application Priority Data
Aug. 17, 2020 (CN) 202010826899.8
Sep. 22, 2020 (CN) 202011004197.8

(51) Int. Cl. C12N 15/86

(52) U.S. Cl. CPC C12N 15/86; C12N 2710/14143; C12N 2710/14144; C12N 2750/14143; C12N 2750/14151

(57) ABSTRACT

A nucleic acid construct for improving an adeno-associated virus yield, and a construction method therefor. The nucleic acid construct includes: an adeno-associated virus (AAV) element, and a polynucleotide encoding an IE protein. Said AAV element includes a polynucleotide encoding a Cap protein, a polynucleotide encoding a Rep protein, and an AAV cis-regulatory element. The construction method includes integrating an AAV element carrying an exogenous target gene and a polynucleotide that encodes the IE protein into to a baculovirus vector backbone. The obtained recombinant adeno-associated virus (rAAV) has a low empty capsid rate, while the rAAV yield of a single cell and a unit volume culture is increased, the production cost is reduced, and the production is easy to scale up.

### TECHNICAL FIELD

The present invention relates to the field of gene transfer vector technology, in particular to a nucleic acid construct for increasing adeno-associated virus (AAV) yield and construction method therefor.

### TECHNICAL BACKGROUND

Recombinant adeno-associated virus (rAAV) is a type of gene transfer vector widely used in basic research and clinical gene therapy. Because of its clinical safety, broad host range, low immunogenicity, and long-lasting expression of exogenous genes in vivo, rAAV has been considered one of the most promising gene transfer vectors and widely used in gene therapy and vaccine research around the world. In recent years, more and more investigators start using rAAV for preclinical studies in large animals and clinical trials. AAV genome comprises three key elements: ITR sequences, coding genes for nonstructural protein Rep, and coding genes for structural protein Cap.

Baculovirus/insect cell system is often used for production of recombinant proteins. Compared with the production using the standard triple-transfection method in HEK293 cells, insect cells can be cultured at high density, serum-free suspension culture, and can be easily scaled up. And, the production method using baculovirus-infected cells is more stable batch to batch. Since 2002, investigators have begun to use baculovirus/insect cell system for the production of rAAVs. However, due to various reasons associated with the production in insect cells, e.g., the ratio of Cap, Rep, and AAV vector genomic DNAs is not easy to reach the ideal ratio, the packaging efficiency of AAV is not high. And, the rAAV vectors that are packaged by traditional methods have a higher empty capsid rate (11 %-34%) (Benskey et al., 2016). Empty AAV capsid is an impurity that must be removed in the preparation of clinical-grade AAV vectors. Because the physical and chemical properties of empty AAV and AAV carrying target gene are very similar, purification is very difficult.

### DESCRIPTION OF THE INVENTION

In view of the above-mentioned shortcomings of the prior art, the purpose of the present invention is to provide a nucleic acid construct and its construction method for improving the yield and quality of the rAAVs, so as to solve the problems in the prior art.

In order to achieve the above purpose and other related purposes, the nucleic acid construct provided by the first aspect of the present invention includes: AAV elements, polynucleotides encoding IE protein, and polynucleotides encoding recombination homologous region of baculovirus, and the AAV elements include a polynucleotide encoding Cap proteins, a polynucleotide encoding Rep proteins, and an AAV cis-acting element.

In some embodiments, the IE protein is encoded by one or more genes of Acie0, Acie01, Acie02, and/or, the recombination homologous region of baculovirus is selected from one or more of hr1, hr2, hr3, hr4, and hr5.

In some embodiments, the nucleic acid construct further includes a promoter of the IE protein gene. In some embodiments, the promoter of the IE protein gene is selected from one or more of Gp64, pH, p6.9, or p 10.

In some embodiments, the nucleic acid construct further includes a baculovirus promoter. In some embodiments, the baculovirus promoter is linked to a recombination homologous region of baculovirus. In some embodiments, the baculovirus promoter is preferably one or more of pH, Gp64, p6.9, or p 10.

In some embodiments, the AAV cis-acting element is selected from ITR sequences.

In some embodiments, the nucleic acid construct further includes an exogenous gene of interest. In some embodiments, the exogenous gene of interest is embedded in between AAV elements.

In some embodiments, the structure of the nucleic acid construct includes: IE gene expression cassette-Cap gene expression cassette-hr1-ITR-exogenous target gene expression cassette-ITR-Rep gene expression cassette. In some embodiments, the structure of the nucleic acid construct includes: IE gene expression cassette-Cap gene expression cassette-ITR-exogenous target gene expression cassette-ITR-Rep gene expression cassette.

In some embodiments, the nucleotide sequence of the nucleic acid construct comprises SEQ ID NO. 1, or comprises nucleotide sequences that share at least 75%, 80%, 85%, 90%, 95%, 96 %, 97%, 98%, or 99% homology with SEQ ID NO.1. In some embodiments, the nucleotide sequence of the nucleic acid construct is shown in SEQ ID NO.1.

In some embodiments, the nucleic acid construct is an AAV vector or a recombinant baculovirus vector. In some embodiments, the nucleic acid construct is an AAV vector. In some embodiments, the nucleic acid construct is a recombinant baculovirus vector. In some embodiments, the ecombinant baculovirus vector is preferably a recombinant baculovirus shuttle vector.

The second aspect of the present invention provides a recombinant baculovirus, and the recombinant baculovirus is obtained by constructing any one of the nucleic acid constructs through the baculovirus system, or is obtained by constructing nucleic acid constructs by including any element of the nucleic acid constructs through the baculovirus system.

The third aspect of the present invention provides an adeno-associated virus, and said AAV is obtained after infection of cells with any one of the recombinant baculoviruses and packaging.

The fourth aspect of the present invention provides a cell line, which is a cell line infected by any one of the recombinant baculoviruses.

The fifth aspect of the present invention provides an AAV vector system, which includes a baculovirus system and the nucleic acid construct.

The sixth aspect of the present invention provides the construction method of the nucleic acid construct, and the construction method includes integrating the AAV elements carrying the exogenous gene of interest, the polynucleotide encoding the IE protein, and the polynucleotide encoding the recombination homologous region of baculovirus into the backbone of the baculovirus vector.

The construction method includes one or more (e.g., two or three) of the following features:
1) The AAV elements include the polynucleotide encoding Cap proteins, the polynucleotide encoding Rep proteins, and AAV cis-acting elements, e.g., the AAV cis-acting elements are preferably ITR sequences;
2) The polynucleotide encoding the IE protein is selected from one or more of Acie0, Acie01, or Acie02;
3) The backbone of the baculovirus vector is selected from one of pFastBacdual, pFastBac1, pFastBacHTA, pFastBacHTB, or pFastBacHTC; or
4) The recombination homologous region of the baculovirus is selected from one or more of hr1, hr2, hr3, hr4, or hr5.

The seventh aspect of the present invention provides a production method for increasing the yield of AAV, and the production method comprises the following steps: infecting insect cell lines with the recombinant baculovirus.

As mentioned above, the nucleic acid construct and its construction method of the present invention for improving the yield of AAV have at least the following beneficial effects:
1) The present invention significantly reduces the expression of the target gene inserted in the baculovirus vector in insect cells, reduces the impact of target gene expression on insect cells; improving the growth index of production cells makes it more conducive to the packaging and production of rAAV.
2) Compared with the production using traditional adherent 293 and 293T cells, as well as conventional production methods using baculovirus, this optimized vector construction method significantly improves rAAV production of single cells and unit volume cultures, and reduces production costs, and the production using this method can be scaled up.
3) A wide range of applications, including the production of AAV gene therapy vectors of various scales and types.

### DESCRIPTION OF DRAWINGS AND FIGURES

FIG. 1 is a schematic diagram showing the structure of the pFBd-Cap-ITR-Rep vector of the present invention.

FIG. 2 is a schematic diagram showing the map of the pFBd-Cap-ITR-Rep vector of the present invention.

FIG. 3 is a schematic diagram showing the structure of the pFBd-IE-hr1Cap-ITR-Rep vector of the present invention.

FIG. 4 is a schematic diagram showing the map of the pFBd-IE-hr1Cap-ITR-Rep vector of the present invention.

FIG. 5 shows the comparison of EGFP expression levels in Sf9 cells 2-4 days after infection with BV-Cap-ITR-Rep and BV-IE-hr1Cap-ITR-Rep of the present invention.

FIG. 6 shows Western blotting detection of lysates of cells infected with two baculoviruses of the present invention using AAV Cap monoclonal antibody.

FIG. 7 shows the electron microscopy examination of rAAV2 of the present invention (titer 2.0E+13 VG/mL).

FIG. 8 shows the fluorescence observation of 293T cells 2 days after transduction with rAAV2 packaged with BV-Cap-ITR-Rep and BV-IE-hr1Cap-ITR-Rep of the present invention.

### DETAILED DESCRIPTION

The present invention is based, at least in part, on the discovery that a nucleic acid construct comprising AAV elements, a polynucleotide encoding an IE protein, and a polynucleotide encoding a recombination homologous region of baculovirus can significantly control the expression of a gene of interest inserted in the baculovirus in its host insect cells, improve the state of cells and the packaging efficiency of AAV, thereby greatly increasing the yield of AAV. The present invention can unexpectedly reduce the empty capsid rate of rAAV (for example, even if the expression level of Cap is increased, it will not lead to an increase in empty capsid), produce rAAV with better infectivity, and significantly improve the yield of rAAV of single cells and unit volume cultures, therefore is suitable for large-scale production of various AAV gene therapy vectors.

Unless otherwise defined below, all technical and scientific terms mentioned herein have the meanings commonly understood by those skilled in the art to which this invention belongs.

The term “nucleic acid construct” refers to an artificially constructed nucleic acid segment that can be introduced into target cells or tissues, and the nucleic acid construct can be a lentiviral vector or an AAV vector, which includes a vector backbone, i.e., the empty vector and expression framework.

The term “vector” refers to a nucleic acid fragment or polynucleotide fragment used to introduce or transfer one or more nucleic acids or one or more polynucleotides into target cells or tissues. Typically, vectors are used to introduce foreign DNA into another cell or tissue. The vector may contain a bacterial resistance gene for growth in bacteria and a promoter for expressing a protein of interest in an organism. DNA can be produced in vitro by PCR or any other suitable technique or techniques known to those skilled in the art.

The first aspect of the present invention provides a nucleic acid construct, said nucleic acid construct comprising: AAV elements and a polynucleotide encoding an IE protein, and the AAV elements comprising a polynucleotide encoding Cap proteins, a polynucleotide encoding Rep proteins, and AAV cis-acting elements.

In one embodiment, the nucleic acid construct further includes a polynucleotide encoding a recombination homologous region of baculovirus.

In some embodiments, the nucleotide sequence of the nucleic acid construct comprises SEQ ID NO.1, or comprises nucleotide sequences that share at least 75%, 80%, 85%, 90%, 95%, 96 %, 97%, 98%, or 99% homology to SEQ ID NO.1. In some embodiments, the nucleotide sequence of the nucleic acid construct is shown in SEQ ID NO.1.

SEQ ID NO: 1:TTCTCTGTCACAGAATGAAAATTTTTCTGTCATCTCT
TCGTTATTAATGTTTGTAATTGACTGAATATCAACGCTTATTTGCAGCCT
GAATGGCGAATGGGACGCGCCCTGTAGCGGCGCATTAAGCGCGGCGGGTG
TGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGT
GGGCCATCGCCCTGATAGACGGTTTTTCGCCCTTTGACGTTGGAGTCCAC
GTTCTTTAATAGTGGACTCTTGTTCCAAACTGGAACAACACTCAACCCTA
TCTCGGTCTATTCTTTTGATTTATAAGGGATTTTGCCGATTTCGGCCTAT
TGGTTAAAAAATGAGCTGATTTAACAAAAATTTAACGCGAATTTTAACAA
AATATTAACGTTTACAATTTCAGGTGGCACTTTTCGGGGAAATGTGCGCG
GAACCCCTATTTGTTTATTTTTCTAAATACATTCAAATATGTATCCGCTC
ATGAGACAATAACCCTGATAAATGCTTCAATAATATTGAAAAAGGAAGAG
TATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAA
CAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGAC
GCCGGGCAAGAGCAACTCGGTCGCCGCATACACTATTCTCAGAATGACTT
GGTTGAGTACTCACCAGTCACAGAAAAGCATCTTACGGATGGCATGACAG
TAAGAGAATTATGCAGTGCTGCCATAACCATGAGTGATAACACTGCGGCC
AACTTACTTCTGACAACGATCGGAGGACCGAAGGAGCTAACCGCTTTTTT
GCACAACATGGGGGATCATGTAACTCGCCTTGATCGTTGGGAACCGGAGC
TGAATGAAGCCATACCAAACGACGAGCGTGACACCACGATGCCTGTAGCA
ATGGCAACAACGTTGCGCAAACTATTAACTGGCGAACTACTTACTCTAGC
TTCCCGGCAACAATTAATAGACTGGATGGAGGCGGATAAAGTTGCAGGAC
CACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAA
CTATGGATGAACGAAATAGACAGATCGCTGAGATAGGTGCCTCACTGATT
AAGCATTGGTAACTGTCAGACCAAGTTTACTCATATATACTTTAGATTGA
TTTAAAACTTCATTTTTAATTTAAAAGGATCTAGGTGAAGATCCTTTTTG
ATAATCTCATGACCAAAATCCCTTAACGTGAGTTTTCGTTCCACTGAGCG
TCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTT
AGGCCACCACTTCAAGAACTCTGTAGCACCGCCTACATACCTCGCTCTGC
TAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTG
AACGGGGGGTTCGTGCACACAGCCCAGCTTGGAGCGAACGACCTACACCG
AACTGAGATACCTACAGCGTGAGCATTGAGAAAGCGCCACGCTTCCCGAA
GGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCC
CTGATTCTGTGGATAACCGTATTACCGCCTTTGAGTGAGCTGATACCGCT
CGCCGCAGCCGAACGACCGAGCGCAGCGAGTCAGTGAGCGAGGAAGCGGA
AGAGCGCCTGATGCGGTATTTTCTCCTTACGCATCTGTGCGGTATTTCAC
ACCGCAGACCAGCCGCGTAACCTGGCAAAATCGGTTACGGTTGAGTAATA
AATGGATGCCCTGCGTAAGCGGGTGTGGGCGGACAATAAAGTCTTAAACT
GAACAAAATAGATCTAAACTATGACAATAAAGTCTTAAACTAGACAGAAT
AGTTGTAAACTGAAATCAGTCCAGTTATGCTGTGAAAAAGCATACTGGAC
TTTTGTTATGGCTAAAGCAAACTCTTCATTTTCTGAAGTGCAAATTGCCC
GTCGTATTAAAGAGGGGCGTGGCCAAGGGCATGGTAAAGACTATATTCGC
GGCGTTGTGACAATTTACCGAACAACTCCGCGGCCGGGAAGCCGATCTCG
GCTTGAACGAATTGTTAGGTGGCGGTACTTGGGTCGATATCAAAGTGCAT
CACTTCTTCCCGTATGCCCAACTTTGTATAGAGAGCCACTGCGGGATCGT
CACCGTAATCTGCTTGCACGTAGATCACATAAGCACCAAGCGCGTTGGCC
TCATGCTTGAGGAGATTGATGAGCGCGGTGGCAATGCCCTGCCTCCGGTG
CTCGCCGGAGACTGCGAGATCATAGATATAGATCTCACTACGCGGCTGCT
CAAACCTGGGCAGAACGTAAGCCGCGAGAGCGCCAACAACCGCTTCTTGG
TCGAAGGCAGCAAGCGCGATGAATGTCTTACTACGGAGCAAGTTCCCGAG
GTAATCGGAGTCCGGCTGATGTTGGGAGTAGGTGGCTACGTCTCCGAACT
CACGACCGAAAAGATCAAGAGCAGCCCGCATGGATTTGACTTGGTCAGGG
CCGAGCCTACATGTGCGAATGATGCCCATACTTGAGCCACCTAACTTTGT
TTTAGGGCGACTGCCCTGCTGCGTAACATCGTTGCTGCTGCGTAACATCG
TTGCTGCTCCATAACATCAAACATCGACCCACGGCGTAACGCGCTTGCTG
CTTGGATGCCCGAGGCATAGACTGTACAAAAAAACAGTCATAACAAGCCA
TGAAAACCGCCACTGCGCCGTTACCACCGCTGCGTTCGGTCAAGGTTCTG
GACCAGTTGCGTGAGCGCATACGCTACTTGCATTACAGTTTACGAACCGA
ACAGGCTTATGTCAACTGGGTTCGTGCCTTCATCCGTTTCCACGGTGTGC
GTCACCCGGCAACCTTGGGCAGCAGCGAAGTCGAGGCATTTCTGTCCTGG
CTGGCGAACGAGCGCAAGGTTTCGGTCTCCACGCATCGTCAGGCATTGGC
GGCCTTGCTGTTCTTCTACGGCAAGGTGCTGTGCACGGATCTGCCCTGGC
TTCAGGAGATCGGTAGACCTCGGCCGTCGCGGCGCTTGCCGGTGGTGCTG
ACCCCGGATGAAGTGGTTCGCATCCTCGGTTTTCTGGAAGGCGAGCATCG
TTTGTTCGCCCAGGACTCTAGCTATAGTTCTAGTGGTTGGCCTACGTACC
CGTAGTGGCTATGGCAGGGCTTGCCGCCCCGACGTTGGCTGCGAGCCCTG
GGCCTTCACCCGAACTTGGGGGTTGGGGTGGGGAAAAGGAAGAAACGCGG
GCGTATTGGTCCCAATGGGGTCTCGGTGGGGTATCGACAGAGTGCCAGCC
CTGGGACCGAACCCCGCGTTTATGAACAAACGACCCAACACCCGTGCGTT
TTATTCTGTCTTTTTATTGCCGTCATAGCGCGGGTTCCTTCCGGTATTGT
CTCCTTCCGTGTTTCAGTTAGCCTCCCCCATCTCCCGGTACCGCATGCCT
TAGTTGAACTCGAACTTCTTGTACTTGCAGTTGAGCTTTTGCTCGGCGAA
TGTGATGGCGTCGGAGAGTGGCACCAGTCCCTGCAGGATCAAGGCCAAGA
GCTTCAGCAAGTTGTTGTGCAGTGTAGTTGACTCGCGACGGTTGACCTTG
CCGATCACGAACATGTGGTGCTTGAATCTGTTGTACTTTTGGATGACCTG
GCTCACATCAACGTCCTTGATTTCGCCAGAGATCCAGTAGAACTCCTTGT
TCTTCTTAGCGATGGTCAGCCTTTCCTCGTTCTTGAAGGACAACACGATG
AAGTTGTGACCCTTAACGTTATCGACGTTTTGAGTCAAGTACTGCTCAAC
GATGTGCATGCTTCCGTCTTCCTTCTTGACCTTCTTGAGGTTCTCGGCGT
TGTTGTTAGAAGCGATGTTGTCGTGGTACTTGTAGTTGTTGAAGAGCAGG
TTAGCGACGGACGAGTACTTGTATGTCAGAGTGCTCTTCTTGTTCACGAT
GAGGTTCAGGTTATCAACGACGTACTTGTTGGGAGGGTTGTCCGGGAATT
GGACGGACTCCGAGTACTTGAGGATCTGGGAAACGTATGGCGAGACGAAG
AAGTTGTTAGAGGCGGTTTCGATCTCGTTTGATTCCTTGCGGCTCAGCAT
GATGGGCAAAGTGAAGAGGTTCTTGTCCTGGTACATCTCGTACAACTTCG
ACAAGAGGAATCCACACTTACGCTCGCCGAGTGATTGCAGCAAGGTCACG
AAGGTTGTCTGAGCGTAGTACATATCGAGGTTGAAGTAGGAGGTCAGAGC
GGCCTTGAATGTGTGGTGGACGTCCACGAAGTGACACTTCTTGCAGTTTT
GAGCGGCGGTCTCATCGTTGCAGACGTCCTGTGAGTGTGGGATTTCGATG
CCAGTCTCCTTAACCAGGTTGTAGCTGATCATGAAGCGGATCTTATCGAA
GGTCACAACGAACACACGGTTGTCAACCATGTAGTAGTTGTTTGTGTACT
CGTAGACCACGTTAGAGACGTACTTGGCGAAGATGATTTCGAAAGGCTTC
ACCTCGGACTTCTTAACGACGAACATGTAGTAACCGGTTTCAGACATGTG
ATCGGAGAATCTGTTCGAGTTGTAGTCGTTATCGTCGAACCTCATCAGGT
AGGGGGCGAAGTCGTTTGTGAAGTAGTGAGTGATCTCCTGGGTGGAAGCC
ACTGTACAGATGTTAGTGTTGTGGTTGATGGTTTGTTCCAGTGTAGCGCA
TGACTGGATGGTGCTCTTCTTGTACTTAGGTCTCAACTTGATCTTGTTGA
ATTGACCCACAACTCCCTGGGAGTTATCCAGGTACTCGTCCAACTTCCTC
TTGGTGCCTGTGGCCGACGGCTGGTTGACACCAGCAGAGTGTTCGAAGGA
CTCGGCGTGGTAAGCTGAGCTAGGAGAGGGTTGTTCGACCACTGGCTGTT
CGAGTGACTCGCTGTAGTAGGCAGAGGACACAGCTTCCTCCAGGTTGTCA
GTGGTCTTGAGCAGACACTCCACCAAATCGTTGTCAGTGAGCGAGTTAAC
TGAGGCGAGGAAGTTGCTAGCGGCAGCGGTTTCAGAGTCGGAGATGACAG
TATCAGCTCCGTCCGGGGTTGGGTGGTTGTAGTAAGACAAGTAATCGTTA
GGTTGCTTGTCGCAGAACTCCGAGTATGAGTTGTCGAAGCTAGCACGAGA
GGGAGTGCTGGCAGAGGTGTAGGAAGCGTTGAAGTTGATTTGAGTCATGG
TGACCTGGTTGTTCACGATCTTATCGCCACCTGTGTCCACCTGCAGTTGC
TGGGCCTCAGCGCAGGCTGAAGTGGCCTCACAGGAGTAGGGGCTGGAAGC
ACAGTTGGAAGTCATGATGTTTTCTTGGACGTTCAGGACGTGGCTGGATG
TACGGATCATGCGCCCGATGGTGGGACGGTATGAATAATCCGGAATATTT
ATAGGTTTTTTTATTACAAAACTGTTACGAAAACAGTAAAATACTTATTT
ATTTGCGAGATGGTTATCATTTTAATTATCTCCATGATCGGATCTCCTAG
GCTCAAGCAGTGATCAGATCCAGACATGATAAGATACATTGATGAGTTTG
GACAAACCACAACTAGAATGCAGTGAAAAAAATGCTTTATTTGTGAAATT
TGTGATGCTATTGCTTTATTTGTAACCATTATAAGCTGCAATAAACAAGT
TAACAACAACAATTGCATTTGTCTAGTACTTCTCGACGGTACCGCATGCT
ATGCATCAGCTGCTAGCACCATGGCTCGAGTTACAGATTACGAGTCAGGT
ATCTGGTGCCAATGGGGCGAGGCTCTGAATACACGCCATTAGTGTCCACA
GTAAAGTCCACATTAACAGACTTGTTGTAGTTGGAAGTGTACTGAATTTC
GGGATTCCAGCGTTTGCTGTTTTCCTTCTGCAGCTCCCACTCGATCTCCA
CGCTGACCTGTCCCGTGGAGTACTGTGTGATGAAGGAAGCAAACTTTGCC
GCACTGAAGGTGGTCGAAGGATTCGCAGGTACCGGGGTGTTCTTGATGAG
AATCTGTGGAGGAGGGTGTTTAAGTCCGAATCCACCCATGAGGGGAGAGG
GGTGAAAATGTCCGTCCGTGTGTGGAATCTTTGCCCAGATGGGCCCCTGA
AGGTACACATCTCTGTCCTGCCAGACCATGCCTGGAAGAACGCCTTGTGT
GTTGACATCTGCGGTAGCTGCTTGTCTGTTGCCTCTCTGGAGGTTGGTAG
ATACAGAACCATACTGCTCCGTAGCCACGGGATTGGTTGTCCTGATTTCC
TCTTCGTCTGTAATCATGACCTTTTCAATGTCCACATTTGTTTTCTCTGA
GCCTTGCTTCCCAAAGATGAGAACCCCGCTCTGAGGAAAAAACTTTTCTT
CATCGTCCTTGTGGCTTGCCATCGCCGGGCCCGGATTCACCAGAGAGTCT
CTGCCATTGAGGTGGTACTTGGTAGCTCCAGTCCACGAGTATTCACTGTT
GTTGTTATCCGCAGATGTCTTTGATACTCGCTGCTGGCGGTAACAGGGTC
CAGGAAGCCAGTTCCTAGACTGGTCCCGAATGTCACTCGCTCCGGCCTGA
GAAAACTGAAGCCTTGACTGCGTGGTGGTTCCACTTGGAGTGTTTGTTCT
GCTCAAGTAATACAGGTACTGGTCGATGAGAGGATTCATGAGACGGTCCA
GACTCTGGCTGTGAGCGTAGCTGCTGTGGAAAGGAACGTCCTCAAAAGTG
TAGCTGAAGGTAAAGTTGTTTCCGGTACGCAGCATCTGAGAAGGAAAGTA
CTCCAGGCAGTAAAATGAAGAGCGTCCTACTGCCTGACTCCCGTTGTTCA
GGGTGAGGTATCCATACTGTGGCACCATGAAGACGTCTGCTGGGAACGGC
GGGAGGCATCCTTGATGCGCCGAGCCGAGGACGTACGGGAGCTGGTACTC
CGAGTCAGTAAACACCTGAACCGTGCTGGTAAGGTTATTGGCAATCGTCG
TCGTACCGTCATTCTGCGTGACCTCTTTGACTTGAATGTTAAAGAGCTTG
AAGTTGAGTCTCTTGGGTCGGAATCCCCAGTTGTTGTTGATGAGTCTTTG
CCAGTCACGTGGTGAAAAGTGGCAGTGGAATCTGTTGAAGTCAAAATACC
CCCAAGGGGTGCTGTAGCCAAAGTAGTGATTGTCGTTCGAGGCTCCTGAT
TGGCTGGAAATTTGTTTGTAGAGGTGGTTGTTGTAGGTGGGCAGGGCCCA
GGTTCGGGTGCTGGTGGTGATGACTCTGTCGCCCATCCATGTGGAATCGC
AATGCCAATTTCCCGAGGAATTACCCACTCCGTCGGCGCCCTCGTTATTG
TCTGCCATTGGTGCGCCACTGCCTGTAGCCATCGTATTAGTTCCCAGACC
AGAGGGGGCTGCTGGTGGCTGTCCGAGAGGCTGGGGGTCAGGTACTGAGT
CTGCGTCTCCAGTCTGACCAAAATTCAATCTTTTTCTTGCAGGCTGCTGG
CCCGCCTTTCCGGTTCCCGAGGAGGAGTCTGGCTCCACAGGAGAGTGCTC
TACCGGCCTCTTTTTTCCCGGAGCCGTCTTAACAGGTTCCTCAACCAGGC
CCAGAGGTTCAAGAACCCTCTTTTTCGCCTGGAAGACTGCTCGTCCGAGG
TTGCCCCCAAAAGACGTATCTTCTTTAAGGCGCTCCTGGAACTCCGCGTC
GGCGTGGTTGTACTTGAGGTAAGGGTTGTCTCCGCTGTCGAGCTGCCGGT
CGTAGGCTTTGTCGTGCTCGAGGGCAGCGGCGTCTGCCTCGTTCACCGGC
TCTCCCTTGTCCAGTCCGTTGAAGGGTCCGAGGTACTTGTACCCGGGAAG
CACAAGGCCCCTGCTGTCGTCCTTATGCCGCTCTGCGGGCTTTGGTGGTG
GTGGGCCGGGTTTGAGCTTCCACCACTGTCTTATTCCTTCAGAGAGAGTG
TCCTCGAGCCAATCGGGTAGATAACCGTCGGCAGCCGTGGCGGCTCAAGT
CTTCGTCGAGTGATTGTAAATAAAATGTAATTTACAGTATAGTATTTTAA
TTAATATACAAATGATTTGATAATAATTCTTATTTAACTATAATATATTG
TGTTGGGTTGAATTAAAGGTCCGTATCTCCAGACTGCAGTTTAAATTGTG
TAATTTATGTAGCTGTAATTTTTACCTTATTAATATTTTTTACGCTTTGC
ATTCGACGACTGAACTCCCAAATATATGTTTAACTCGTCTTGGTCGTTTG
AATTTTTGTTGCTGTGTTTCCTAATATTTTCCATCACCTTAAATATGTTA
TTGTAATCCTCAATGTTGAACTTGCAATTGGACACGGCATAGTTTTCCAT
AGTCGTGTAAAACATGGTATTGGCTGCATTGTAATACATCCGACTGAGCG
GGTACGGATCTATGTGTTTGAGCAGCCTGTTCAAAAACTCTGCATCGTCG
CAAAACGGAATTTGGTCACGATCCCGGTCCGAAGCGCGCGGAATTCAAAG
CGCTACGTCAGCGACGTCATCGATTATTGCTCCAATACTAGGGTTTTGTT
TTACTATCTGTTCTGCGGTCCACACATCTTCTTCGCTAGTCACGTACGCA
AACAACTCGGGCCGCAAAGGTGTTTTAAAACATATCAGATTAGAATCTTT
TATAACTTGGCCGCATTGTAAATAGTTGTGCCAACGCGCGGGAAACATTT
TACACGTGCTTTCAATCTTATCTCATAAACGCAACTAAATATGATCCTTC
AACTCGCTTTACGAGTAGAATTCTACTTGTAACGCACGATTAATTATGAC
TCATAAGCTGATGTCATGTTTTGCACACGGCTCATAACCGAACTCGCTTT
ACGAGTAGAATTCTACGCGTAAAACACAATCAAGTATGAGTCATAAGCTG
ATGTCATGTTTTGCACACGGCTCATAACCGAACTGGCTTTACGAGTAGAA
TTCTACTTGTAACGCACGATCAGTGGATGATGTCATAAACTTATCTCATG
TTTTGCACACGGCTCATAACTTGAGTCATAAGCTGATATCAGAAGATTAT
TAACGTGTTTAGGTATGACTCATTTGTTTTCAAAACTGAACTCGCTTTAC
GGATAGAATTCTACTTGTAAAACACAATCAGGGGATGATGTCATTATACA
AATGATGTCATTTGTTTTTCAAAACTAAACTCGCTTTACGGGTAGAATTC
TACTTGTAAAACACAATCGAGGGATAATGTCATTTGTTATTCTCAAATAT
GATGTCATTTAAATTTTTATTATGCAAGAATTGTATGTTGCTGTGTCATT
TATGAATGTTTTATCGACAACTTGTTTATTTGTTTCATTGACTTGTTGTT
GTTCGTGATTATGATTAATTATAAATCTTTTGTTGGGGTCAACATCGATG
TATACGCCGCTACAGGGCGCGTACTATGGTTGCTTTGACGTATGCGGTGT
GAAATACCGCACAGATGCGTAAGGAGAAAATACCGCATCAGGCGCCCCTG
CAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGC
AGAGAGGGAGTGGCCAACTCCATCACTAGGGGTTCCTACGCGTTAGTTAT
TAATAGTAATCAATTACGGGGTCATTAGTTCATAGCCCATATATGGAGTT
CCGCGTTACATAACTTACGGTAAATGGCCCGCCTGGCTGACCGCCCAACG
ACCCCCGCCCATTGACGTCAATAATGACGTATGTTCCCATAGTAACGCCA
ATAGGGACTTTCCATTGACGTCAATGGGTGGAGTATTTACGGTAAACTGC
CCACTTGGCAGTACATCAAGTGTATCATATGCCAAGTACGCCCCCTATTG
ACGTCAATGACGGTAAATGGCCCGCCTGGCATTATGCCCAGTACATGACC
TTATGGGACTTTCCTACTTGGCAGTACATCTACGTATTAGTCATCGCTAT
TACCATGGTGATGCGGTTTTGGCAGTACATCAATGGGCGTGGATAGCGGT
TTGACTCACGGGGATTTCCAAGTCTCCACCCCATTGACGTCAATGGGAGT
TTGTTTTGGCACCAAAATCAACGGGACTTTCCAAAATGTCGTAACAACTC
CGCCCCATTGACGCAAATGGGCGGTAGGCGTGTACGGTGGGAGGTCTATA
TAAGCAGAGCTGGTTTAGTGAACCGTCAGATCCGCTAGCGCTACCGGTCG
CCACCGTCGACGTTTATTTGTGAAATTTGTGATGCTATTGCTTTATTTGT
AACCATCTAGCTTTATTTGTGAAATTTGTGATGCTATTGCTTTATTTGTA
ACCATTATAAGCTGCAATAAACAAGTTAACAACAACAATTGCATTCATTT
TATGTTTCAGGTTCAGGGGGAGATGTGGGAGGTTTTTTAAAGTTTAAACA
GGAACCCCTAGTGATGGAGTTGGCCACTCCCTCTCTGCGCGCTCGCTCGC
TCACTGAGGCCGGGCGACCAAAGGTCGCCCGACGCCCGGGCGGCCTCAGT
GAGCGAGCGAGCGCGCAGCTGCCTGCAGGACATGTGAGCAAAAGGCCAGC
AAAAGGCCAGGAACCGTAAAAAGGCCGCGTTGCTGGCGTTTTTCCATAGG
CTCCGCCCCCCTGACGAGCATCACAAAAATCGACGCTCGCGGCCGCTCTA
GAGATCATGGAGATAATTAAAATGATAACCATCTCGCAAATAAATAAGTA
TTTTACTGTTTTCGTAACAGTTTTGTAATAAAAAAACCTATAAATATTCC
GGATTATTCATACCGTCCCACCATCGGGCGCGGATCCGCCGCCCTGGCGG
GGTTTTACGAGATTGTGATTAAGGTCCCCAGCGACCTTGACGAGCATCTG
CCCGGCATTTCTGACAGCTTTGTGAACTGGGTGGCCGAGAAGGAGTGGGA
GTTGCCGCCAGATTCTGACTTGGATCTGAATCTGATTGAGCAGGCACCCC
TGACCGTGGCCGAGAAGCTGCAGCGCGACTTTCTGACGGAGTGGCGCCGT
GTGAGTAAGGCCCCGGAGGCCCTTTTCTTTGTGCAATTTGAGAAGGGAGA
GAGCTACTTCCACTTACACGTGCTCGTGGAAACCACCGGGGTGAAATCCT
TAGTTTTGGGACGTTTCCTGAGTCAGATTCGCGAAAAACTGATTCAGAGA
ATTTACCGCGGGATCGAGCCGACTTTGCCAAACTGGTTCGCGGTCACAAA
GACCAGAAACGGCGCCGGAGGCGGGAACAAGGTGGTGGACGAGTGCTACA
TCCCCAATTACTTGCTCCCCAAAACCCAGCCTGAGCTCCAGTGGGCGTGG
ACTAATTTAGAACAGTATTTAAGCGCCTGTTTGAATCTCACGGAGCGTAA
ACGGTTGGTGGCGCAGCATCTGACGCACGTGTCGCAGACGCAGGAGCAGA
ACAAAGAGAATCAGAATCCCAATTCTGACGCGCCGGTGATCAGATCAAAA
ACTTCAGCCAGGTACATGGAGCTGGTCGGGTGGCTCGTGGACAAGGGGAT
TACCTCGGAGAAGCAGTGGATACAGGAGGACCAGGCCTCATACATCTCCT
TCAATGCGGCCTCCAACTCGCGGTCCCAAATCAAGGCTGCCTTGGACAAT
GCGGGAAAGATTATGAGCCTGACTAAAACCGCCCCCGACTACCTGGTGGG
CCAGCAGCCCGTGGAGGACATTTCCAGCAATCGGATTTATAAAATTTTGG
AACTAAACGGGTACGATCCCCAATATGCGGCTTCCGTCTTTCTGGGATGG
GCCACGAAAAAGTTCGGCAAGAGGAACACCATCTGGCTGTTTGGGCCTGC
AACTACCGGGAAGACCAACATCGCGGAGGCCATAGCCCACACTGTGCCCT
TCTACGGGTGCGTAAACTGGACCAATGAGAACTTTCCCTTCAACGACTGT
GTGGACAAGATGGTGATCTGGTGGGAGGAGGGGAAGATGACCGCCAAGGT
CGTGGAGTCGGCCAAAGCCATTCTCGGAGGAAGCAAGGTGCGCGTGGACC
AGAAATGCAAGTCCTCGGCCCAGATAGACCCGACTCCCGTGATCGTCACC
TCCAACACCAACATGTGCGCCGTGATTGACGGGAACTCAACGACCTTCGA
ACACCAGCAGCCGTTGCAAGACCGGATGTTCAAATTTGAACTCACCCGCC
GTCTGGATCATGACTTTGGGAAGGTCACCAAGCAGGAAGTCAAAGACTTT
TTCCGGTGGGCAAAGGATCACGTGGTTGAGGTGGAGCATGAATTCTACGT
CAAAAAGGGTGGAGCCAAGAAAAGACCCGCCCCCAGTGACGCAGATATAA
GTGAGCCCAAACGGGTGCGCGAGTCAGTTGCGCAGCCATCGACGTCAGAC
GCGGAAGCATCGATCAACTACGCAGACAGGTACCAAAACAAATGTTCTCG
TCACGTGGGCATGAATCTGATGCTGTTTCCCTGCAGACAATGCGAGAGAA
TGAATCAGAATTCAAATATCTGCTTCACTCACGGACAGAAAGACTGTTTA
GAGTGCTTTCCCGTGTCAGAATCTCAACCCGTTTCTGTCGTCAAAAAGGC
GTATCAGAAACTGTGCTACATTCATCATATCATGGGAAAGGTGCCAGACG
CTTGCACTGCCTGCGATCTGGTCAATGTGGATTTGGATGACTGCATCTTT
GAACAATAATCTAGAGCCTGCAGTCTCGACAAGCTTGTCGAGAAGTACTA
GAGGATCATAATCAGCCATACCACATTTGTAGAGGTTTTACTTGCTTTAA
AAAACCTCCCACACCTCCCCCTGAACCTGAAACATAAAATGAATGCAATT
GTTGTTGTTAACTTGTTTATTGCAGCTTATAATGGTTACAAATAAAGCAA
TAGCATCACAAATTTCACAAATAAAGCATTTTTTTCACTGCATTCTAGTT
GTGGTTTGTCCAAACTCATCAATGTATCTTATCATGTCTGGATCTGATCA
CTGCTTGAGCCTAGGAGATCCGAACCAGATAAGTGAAATCTAGTTCCAAA
CTATTTTGTCATTTTTAATTTTCGTATTAGCTTACGACGCTACACCCAGT
TCCCATCTATTTTGTCACTCTTCCCTAAATAATCCTTAAAAACTCCATTT
CCACCCCTCCCAGTTCCCAACTATTTTGTCCGCCCACAGCGGGGCATTTT
TCTTCCTGTTATGTTTTTAATCAAACATCCTGCCAACTCCATGTGACAAA
CCGTCATCTTCGGCTACTTT

In some embodiments, the AAV elements are derived from AAV of different serotypes, such as AAV1, AAV2, AAV3, AAV4, AAV5, AAV6, AAV7, AAV8, AAV9, AAV DJ, AAV DJ/8, AAV rh10, AAV Retro, AAV PHP.eB, AAV PHP.B, or AAV PHP. S. The capsid proteins of different serotypes of AAV recognize different receptors on the cell surface, so the infection efficiency to different tissue cells may vary greatly, showing certain organ-targeting specificity. The appropriate serotype of AAV can be selected according to the specific experimental purpose, so as to achieve specific high-efficiency transduction of a specific serotype of AAV to a specific type of cell and tissue.

In some embodiments, the AAV elements are derived from AAV1. In some embodiments, the AAV elements are derived from AAV2. In some embodiments, the AAV elements are derived from AAV3. In some embodiments, the AAV elements are derived from AAV4. In some embodiments, the AAV elements are derived from AAV5. In some embodiments, the AAV elements are derived from AAV6. In some embodiments, the AAV elements are derived from AAV7. In some embodiments, the AAV elements are derived from AAV8. In some embodiments, the AAV elements are derived from AAV9. In some embodiments, the AAV elements are derived from AAV DJ. In some embodiments, the AAV elements are derived from AAV DJ/8. In some embodiments, the AAV elements are derived from AAV rh10. In some embodiments, the AAV elements are derived from AAV Retro. In some embodiments, the AAV elements are derived from AAV PHP.eB/PHP.B/PHP.S.

The Cap proteins are structural proteins, usually including VP1, VP2, and VP3 structural proteins. It is believed that in some embodiments, three structural proteins can be assembled into the capsid protein of AAV.

The Rep proteins are non-structural proteins, usually including Rep78 and 52 proteins. It is believed that in some embodiments, Rep proteins regulate the replication and packaging of DNA.

In one embodiment, the AAV cis-acting elements are selected from inverted terminal repeat sequences (ITR sequences). It is believed that in some embodiments, the ITR sequences fold into a hairpin structure and are the only known cis-acting elements required for replication initiation of AAV DNA and packaging of recombinant AAV virions.

In some embodiments, the nucleic acid construct further includes an exogenous gene of interest. In some embodiments, the exogenous target gene is carried by AAV elements, that is, the exogenous target gene is embedded in between the AAV elements.

Specifically, in some embodiments, the exogenous target gene is embedded between two ITR sequences.

Preferably, in some embodiments, the size of the exogenous target gene is ≤3.5 kb. It is believed that in some embodiments, due to the size limitation between the two ITR sequences, the size of the exogenous target gene should generally not be too large, otherwise it may lead to poor packaging effect.

The type of the exogenous gene varies according to the purpose of the experiment. The exogenous gene can be a gene related to the treatment of various diseases, such as AADC, FVIII, FIX, and can also be a tool gene commonly used in laboratories, such as EGFP, mCherry gene, and the like.

It is believed that in some embodiments, the nucleic acid construct also includes a promoter for the IE protein-coding gene. It is believed that in some embodiments, the promoter of the IE protein-coding gene is selected from one or more of Gp64, pH, p6.9, and p 10. In some embodiments, the promoter of the IE protein-coding gene includes Gp64. In some embodiments, the promoter of the IE protein-coding gene comprises pH. In some embodiments, the promoter of the IE protein-coding gene includes p6.9. In some embodiments, the promoter of the IE protein-coding gene includes p 10.

Preferably, in some embodiments, the promoter of the IE protein-coding gene is selected from strong pH promoters. It is believed that in some embodiments, incorporation of an additional copy of ie gene into the nucleic acid construct and expressed under the control of a strong promoter pH above the endogenous IE levels of the virus increases the survival of cells at late stage following baculovirus infection .

In some embodiments, the IE protein is selected from proteins encoded by one or more (e.g., two or three) genes of Acie0, Acie01, and Acie02. In some embodiments, the IE protein is selected from a protein encoded by Acie0, i.e., IE0. The nucleotide sequence of Acie0 is shown in SEQ ID NO.10, and the amino acid sequence of the IE0 protein is shown in SEQ ID NO.11. In some embodiments, the said IE protein is selected from the protein encoded by Acie01, i.e., IE1. The nucleotide sequence of Acie01 is shown in SEQ ID NO.12, and the amino acid sequence of IE1 protein is shown in SEQ ID NO.13. In some embodiments, the IE protein is selected from the protein encoded by Acie02, namely IE2 protein. The nucleotide sequence of Acie02 is shown in SEQ ID NO.14, and the amino acid sequence of IE2 protein is shown in SEQ ID NO.15.

The sequences of SEQ ID NO.10-15 are as follows:

SEQ ID NO.10:ATGATAAGAACCAGCAGTCACGTGCTGAACGTCCAGG
AAAATATAATGACGTCAAACTGTGCGTCATCGCCATATTCGTGCGAGGCA
ACGTCCGCTTGCGCAGAAGCTCAGCAGGTAATGATCGATAACTTTGTTTT
CTTTCACATGTACAACGCCGACATACAAATTGACGCAAAGCTGCAATGCG
GCGTGCGCTCGGCCGCGTTTGCAATGATCGACGATAAACATTTGGAAATG
TACAAGCATAGAATAGAGAATAAATTTTTTTATTACTATGATCAATGTGC
CGACATTGCCAAACCCGACCGTCTGCCCGATGACGACGGCGCGTGCTGTC
ACCATTTTATTTTTGATGCCCAACGTATTATTCAATGTATTAAAGAGATT
GAAAGCGCGTACGGCGTGCGTGATCGCGGCAATGTAATAGTGTTTTATCC
GTACTTGAAACAGTTGCGAGACGCGTTGAAGCTAATTAAAAACTCTTTTG
CGTGTTGTTTTAAAATTATAAATTCTATGCAAATGTACGTGAACGAGTTA
ATATCAAATTGCCTGTTGTTTATTGAAAAGCTGGAAACTATTAATAAAAC
TGTTAAAGTTATGAATTTGTTTGTAGACAATTTGGTTTTGTACGAATGCA
ATGTTTGTAAAGAAATATCTACGGATGAAAGATTTTTAAAGCCAAAAGAA
TGTTGCGAATACGCTATATGCAACGCGTGCTGCGTTAACATGTGGAAGAC
GGCCACCACGCACGCAAAATGTCCAGCGTGCAGGACATCGTATAAATAA

SEQ ID NO.11:MIRTSSHVLNVQENIMTSNCASSPYSCEATSACAEAQ
QVMIDNFVFFHMYNADIQIDAKLQCGVRSAAFAMIDDKHLEMYKHRIENK
FFYYYDQCADIAKPDRLPDDDGACCHHFIFDAQRIIQCIKEIESAYGVRD
RGNVIVFYPYLKQLRDALKLIKNSFACCFKIINSMQMYVNELISNCLLFI
EKLETINKTVKVMNLFVDNLVLYECNVCKEISTDERFLKPKECCEYAICN
ACCVNMWKTATTHAKCPACRTSYK

SEQ ID NO.12:ATGATCCGTACATCCAGCCACGTCCTGAACGTCCAAG
AAAACATCATGACTTCCAACTGTGCTTCCAGCCCCTACTCCTGTGAGGCC
ACTTCAGCCTGCGCTGAGGCCCAGCAACTGCAGGTGGACACAGGTGGCGA
TAAGATCGTGAACAACCAGGTCACCATGACTCAAATCAACTTCAACGCTT
CCTACACCTCTGCCAGCACTCCCTCTCGTGCTAGCTTCGACAACTCATAC
TCGGAGTTCTGCGACAAGCAACCTAACGATTACTTGTCTTACTACAACCA
CCCAACCCCGGACGGAGCTGATACTGTCATCTCCGACTCTGAAACCGCTG
CCGCTAGCAACTTCCTCGCCTCAGTTAACTCGCTCACTGACAACGATTTG
GTGGAGTGTCTGCTCAAGACCACTGACAACCTGGAGGAAGCTGTGTCCTC
TGCCTACTACAGCGAGTCACTCGAACAGCCAGTGGTCGAACAACCCTCTC
CTAGCTCAGCTTACCACGCCGAGTCCTTCGAACACTCTGCTGGTGTCAAC
CAGCCGTCGGCCACAGGCACCAAGAGGAAGTTGGACGAGTACCTGGATAA
CTCCCAGGGAGTTGTGGGTCAATTCAACAAGATCAAGTTGAGACCTAAGT
ACAAGAAGAGCACCATCCAGTCATGCGCTACACTGGAACAAACCATCAAC
CACAACACTAACATCTGTACAGTGGCTTCCACCCAGGAGATCACTCACTA
CTTCACAAACGACTTCGCCCCCTACCTGATGAGGTTCGACGATAACGACT
ACAACTCGAACAGATTCTCCGATCACATGTCTGAAACCGGTTACTACATG
TTCGTCGTTAAGAAGTCCGAGGTGAAGCCTTTCGAAATCATCTTCGCCAA
GTACGTCTCTAACGTGGTCTACGAGTACACAAACAACTACTACATGGTTG
ACAACCGTGTGTTCGTTGTGACCTTCGATAAGATCCGCTTCATGATCAGC
TACAACCTGGTTAAGGAGACTGGCATCGAAATCCCACACTCACAGGACGT
CTGCAACGATGAGACCGCCGCTCAAAACTGCAAGAAGTGTCACTTCGTGG
ACGTCCACCACACATTCAAGGCCGCTCTGACCTCCTACTTCAACCTCGAT
ATGTACTACGCTCAGACAACCTTCGTGACCTTGCTGCAATCACTCGGCGA
GCGTAAGTGTGGATTCCTCTTGTCGAAGTTGTACGAGATGTACCAGGACA
AGAACCTCTTCACTTTGCCCATCATGCTGAGCCGCAAGGAATCAAACGAG
ATCGAAACCGCCTCTAACAACTTCTTCGTCTCGCCATACGTTTCCCAGAT
CCTCAAGTACTCGGAGTCCGTCCAATTCCCGGACAACCCTCCCAACAAGT
ACGTCGTTGATAACCTGAACCTCATCGTGAACAAGAAGAGCACTCTGACA
TACAAGTACTCGTCCGTCGCTAACCTGCTCTTCAACAACTACAAGTACCA
CGACAACATCGCTTCTAACAACAACGCCGAGAACCTCAAGAAGGTCAAGA
AGGAAGACGGAAGCATGCACATCGTTGAGCAGTACTTGACTCAAAACGTC
GATAACGTTAAGGGTCACAACTTCATCGTGTTGTCCTTCAAGAACGAGGA
AAGGCTGACCATCGCTAAGAAGAACAAGGAGTTCTACTGGATCTCTGGCG
AAATCAAGGACGTTGATGTGAGCCAGGTCATCCAAAAGTACAACAGATTC
AAGCACCACATGTTCGTGATCGGCAAGGTCAACCGTCGCGAGTCAACTAC
ACTGCACAACAACTTGCTGAAGCTCTTGGCCTTGATCCTGCAGGGACTGG
TGCCACTCTCCGACGCCATCACATTCGCCGAGCAAAAGCTCAACTGCAAG
TACAAGAAGTTCGAGTTCAACTAA

SEQ ID NO.13:MIRTSSHVLNVQENIMTSNCASSPYSCEATSACAEAQ
QLQVDTGGDKIVNNQVTMTQINFNASYTSASTPSRASFDNSYSEFCDKQP
NDYLSYYNHPTPDGADTVISDSETAAASNFLASVNSLTDNDLVECLLKTT
DNLEEAVSSAYYSESLEQPVVEQPSPSSAYHAESFEHSAGVNQPSATGTK
RKLDEYLDNSQGVVGQFNKIKLRPKYKKSTIQSCATLEQTINHNTNICTV
ASTQEITHYFTNDFAPYLMRFDDNDYNSNRFSDHMSETGYYMFVVKKSEV
KPFEIIFAKYVSNVVYEYTNNYYMVDNRVFVVTFDKIRFMISYNLVKETG
IEIPHSQDVCNDETAAQNCKKCHFVDVHHTFKAALTSYFNLDMYYAQTTF
VTLLQSLGERKCGFLLSKLYEMYQDKNLFTLPIMLSRKESNEIETASNNF
FVSPYVSQILKYSESVQFPDNPPNKYVVDNLNLIVNKKSTLTYKYSSVAN
LLFNNYKYHDNIASNNNAENLKKVKKEDGSMHIVEQYLTQNVDNVKGHNF
IVLSFKNEERLTIAKKNKEFYWISGEIKDVDVSQVIQKYNRFKHHMFVIG
KVNRRESTTLHNNLLKLLALILQGLVPLSDAITFAEQKLNCKYKKFEFN

SEQ ID NO.14:ATGAGTCGCCAAATCAACGCCGCCACTCCCAGCAGCA
GCCGCCGCCACAGGCTGTCTCTCAGCCGTCGCCGCATCAACTTTACAACA
TCTCCCGAAGCCCAGCCGTCTTCAAGCAGTCGCAGCCAGCCGTCTTCAAG
CAGTCGCAGCCATCGCCGTCAGGAGCGGCGTCAGGAGCAGCGTGTCAGCG
AAGAAAACGTGCAGATTATCGGGAACGTCAACGAGCCGTTGACGCGCACC
TACCATCGTCAGGGTGTCACGTATTACGTGCACGGTCAGGTTAACATTAG
CAATGACGATCCGCTATTAAGTCAAGAGGATGACGTCATACTAATTAATA
GTGAAAATGTGGATCGTGAACGGTTTCCCGACATCACTGCCCAGCAGTAC
CAGGATAACATTGCGTCGGAGACAGCTGCGCAGAGGGCTCTGCAACGAGG
TTTAGATCTTGAGGCTCAGCTGATGAATGAGATTGCCCCAAGGTCTCCCA
CTTATAGTCCATCTTATTCGCCGAATTACGTAATACCACAGTCGCCAGAT
TTGTTTGCCTCGCCGCAGTCTCCGCAGCCGCAGCAGCAGCAGCAGCAGCA
ATCAGAACCCGAAGAAGAAGTAGAGGTTTCGTGTAATATTTGTTTTACTA
CTTTTAAAGACACTAAAAACGTAAATTCCTCGTTTGTGACTTCGATTCAT
TGTAACCATGCTGTGTGTTTCAAGTGTTATGTCAAGATAATTATGGACAA
TTCTGTGTACAAATGTTTTTGCAGCGCTACTTCATCAGATTGTCGCGTGT
ACAATAAGCACGGGTATGTAGAATTTATGCCCATTAACGTCACTCGTAAC
CAGGATTCCATCAAACAGCATTGGCGCGAGCTTTTAGAAAATAACACGGT
CAACAATCACACCACGGACTTGAACTATGTGGAGCAATTGCAAAAAGAAC
TGTCCGAGCTGCGAGCCAAGACCAGCCAAGTTGAACATAAAATGACCATG
TTAAACAGCGACTACATTATGCTTAAACACAAGCATGCTGTCGCCGAATT
AGATTTACAAAAGGCAAACTATGACTTGCAAGAATCTACCAAGAAATCAG
AAGAGTTGCAATCGACTGTGAATAATCTGCAAGAACAATTGCGTAAGCAG
GTGGCCGAGTCTCAAGCCAAATTTTCAGAGTTTGAGCGCAGTAACTCTGA
TTTAGTTTCTAAGTTACAAACTGTTATGTCTAGACGTTAA

SEQ ID NO.15:MSRQINAATPSSSRRHRLSLSRRRINFTTSPEAQPSS
SSRSQPSSSSRSHRRQERRQEQRVSEENVQIIGNVNEPLTRTYHRQGVTY
YVHGQVNISNDDPLLSQEDDVILINSENVDRERFPDITAQQYQDNIASET
AAQRALQRGLDLEAQLMNEIAPRSPTYSPSYSPNYVIPQSPDLFASPQSP
QPQQQQQQQSEPEEEVEVSCNICFTTFKDTKNVNSSFVTSIHCNHAVCFK
CYVKIIMDNSVYKCFCSATSSDCRVYNKHGYVEFMPINVTRNQDSIKQHW
RELLENNTVNNHTTDLNYVEQLQKELSELRAKTSQVEHKMTMLNSDYIML
KHKHAVAELDLQKANYDLQESTKKSEELQSTVNNLQEQLRKQVAESQAKF
SEFERSNSDLVSKLQTVMSRR

It is believed that in some embodiments, the IE1 protein is a product of the immediate early gene 1 (ie1) of baculovirus, and the IE protein may also be referred to as a transcriptional regulator protein of baculovirus. It is a multifunctional protein with the ability to activate early and late viral genes and participate in viral genome replication, and participates in the regulation of the viral cycle.

In some embodiments, the recombination homologous region of baculovirus (hr) is selected from one or more (e.g., two, three, four or five) of hr1 (its nucleotide sequence shown in SEQ ID NO.16), hr2 (its nucleotide sequence shown in SEQ ID NO.17), hr3 (its nucleotide sequence is shown in SEQ ID NO.18), hr4 (the nucleotide sequence of hr4 left is shown in SEQ ID NO.19, and the nucleotide sequence of hr4 right is shown in SEQ ID NO.20), and hr5 (its nucleotide sequence is shown in SEQ ID NO.21).

The sequences of SEQ ID NO.16-21 are as follows:

SEQ ID NO.16:ATCGATGTTGACCCCAACAAAAGATTTATAATTAATC
ATAATCACGAACAACAACAAGTCAATGAAACAAATAAACAAGTTGTCGAT
AAAACATTCATAAATGACACAGCAACATACAATTCTTGCATAATAAAAAT
TTAAATGACATCATATTTGAGAATAACAAATGACATTATCCCTCGATTGT
GTTTTACAAGTAGAATTCTACCCGTAAAGCGAGTTTAGTTTTGAAAAACA
AATGACATCATTTGTATAATGACATCATCCCCTGATTGTGTTTTACAAGT
AGAATTCTATCCGTAAAGCGAGTTCAGTTTTGAAAACAAATGAGTCATAC
CTAAACACGTTAATAATCTTCTGATATCAGCTTATGACTCAAGTTATGAG
CCGTGTGCAAAACATGAGATAAGTTTATGACATCATCCACTGATCGTGCG
TTACAAGTAGAATTCTACTCGTAAAGCCAGTTCGGTTATGAGCCGTGTGC
AAAACATGACATCAGCTTATGACTCATACTTGATTGTGTTTTACGCGTAG
AATTCTACTCGTAAAGCGAGTTCGGTTATGAGCCGTGTGCAAAACATGAC
ATCAGCTTATGAGTCATAATTAATCGTGCGTTACAAGTAGAATTCTACTC
GTAAAGCGAGTTGAAGGATCATATTTAGTTGCGTTTATGAGATAAGATTG
AAAGCACGTGTAAAATGTTTCCCGCGCGTTGGCACAACTATTTACAATGC
GGCCAAGTTATAAAAGATTCTAATCTGATATGTTTTAAAACACCTTTGCG
GCCCGAGTTGTTTGCGTACGTGACTAGCGAAGAAGATGTGTGGACCGCAG
AACAGATAGTAAAACAAAACCCTAGTATTGGAGCAATAATCGAT

SEQ ID NO.17:TGAGCAAAACACAACCGGCAAATTCTCGGCGGCCGTT
TGGGAATGCGGAATAATTGCCATATGTAAATGATGTCATCGGTTCTAACT
CGCTTTACGAGTAGAATTCTACGTGTAAAACATAATCAAGAGATGATGTC
ATTTGTTTTTCAAAACTGAACTCAAGAAATGATGTCATTTGTTTTTCAAA
ACTGAACTGGCTTTACGAGTAGAATTCTACTTGTAAAACACAATCGAGAG
ATGATGTCATATTTTGCACACGGCTCTAATTAAACTCGCTTTACGAGTAA
AATTCTACTTGTAACGCATGATCAAGGGATGATGTCATTGGATGAGTCAT
TTGTTTTTCAAAACTAAACTCGCTTTACGAGTAGAATTCTACTTGTAAAA
CACAATCAAGGGATGATGTCATTATACAAATGATGTCATTTGTTTTTCAA
AACTAAACTCGCTTTACGGGTAGAATTCTACTTGTAAAACAGCAACTCGA
GGGATGATGTCATCCTTTACTCGATGATTATAAACGTGTTTATGTATGAC
TCATTTGTTTTTCAAAACTAAACTCGCTTTACGAGTAGAATTCTACTTGT
AACGCACGATCAAGGGATGATGTCATTTATTTGTGCAAAGCTCGATGTCA
TCTTTTGCACACGATTATAAACACAATCCAAATAATGACTCATTTGTTTT
CAAAACTGAACTCGCTTTACGAGTAGAATTCTACTTGTAAAACACAATCA
AGGGATGATGTCATTTTCAAAATGATGTCATTTGTTTTTCAAAACTAAAC
TCGCTTTACGAGTAGAATTCTACTTGTAAAACACAATCAAGGGATGATGT
CATTTTAAAAATGATCATTTGTTTTTCAAAACTAAACTCGCTTTACGAGT
AGAATTCTACGTGTAAAACACAATCAAGGGATGATGTCATTTACTAAATA
AAATAATTATTTAAATAAAACTGTTTTTTATTGTCAAATACACATTGATT
CAC

SEQ ID NO.18:ACGCGTAGAATTCTACTTGTAAAGCAAGTTAAAATAA
GCCGTGTGCAAAAATGACATCAGACAAATGACATCATCTACCTATCATGA
TCATGTTAATAATCATGTTTTAAAATGACATCAGCTTATGACTAATAATT
GATCGTGCGTTACAAGTAGAATTCTACTCGTAAAGCGAGTTTAGTTTTGA
AAACAAATGAGTCATCATTAAACATGTTAATAATCGTGTATAAAGGATGA
CATCATCCACTAATCGTGCGTTACAAGTAGAATTCTACTCGTAAAGCGAG
TTCGGTTTTGAAAAACAAATGACATCATTTCTTGATTGTGTTTTACACGT
AGAATTCTACTCGTAAAGTATGTTCAGTTTAAAAAACAAATGACATCATT
TTACAGATGACATCATTTCTTGATTATGTTTTACAAGTAGAATTCTACTC
GTAAAGCGAGTTTAGTTTTAAAAAACAAATGACATCATCTCTTGATTATG
TTTTACAAGTAGAATTCTACTCGTAAAGCGAGTTTAGTTTTGAAAAACAA
ATGACATCATCTCTTGATTATGTTTTACAAGTAGAATTCTACTCGTAAAG
CGAGTTTAGTTTTGAAAAACAAATGACATCATCCCTTGATCATGCGTTAC
AAGTAGAATTCTACTCGTAAAGCGAGTTGAATTTTGATTACAATATT

SEQ ID NO.19:ATGCATATAATTGTGTACAAAATATGACTCATTAATC
GATCGTGCGTTACAAGTAGAATTCTACTGGTAAAGCAAGTTCGGTTGTGA
GCCGTGTGCAAAACATGACATCATAACTAATCATGTTTATAATCATGTGC
AAAATATGACATCATCCGACGATTGTGTTTTACAAGTAGAATTCTACTCG
TAAAGCGAGTTTAAAAATTTTGTGACGTCAATGAAACAACGTGTAATATT
TTTTACAATATTTAAGTGAAACATTATGACTTCCAATAATTTTGTGGATG
TGGATACGTTTGCAAGACAATTGATTACAGATAAATGTAGTGCTCTAATC
GAAAGATGCGGATCTGTTGCCGGCAAACATTTTAGAGATTAGTAGAGAAA
GGCCAGAGACAAGTATTTTGAGGTGCCAACTCAAAAAAACTATGAATACA
TTAAAAAATTATTTTTACGAACAAAATATATGGACGATTCGATAGATTAT
AAAGATTTTAACAGACGCATCCTATTGATAGTTTTTAAATTCGCTTTAAA
CAAGAGCACCAACTACTTTCCATCGTACTAAAGAGATCATCGAGGTGGCC
ATTAAACGTTTAAACAAAATTAACCCCGATTTAAAGAGTTCTCCGCGCAA
TGCTTCAGCATTACAAATGAATGTTTGGAAAATCTAGA

SEQ ID NO.20:AACTGGCTTTACGAGTAGAATTCTACTTGTAAAACAC
AATCAAGAAATGATGTCATTTTTGTACGTGATTATAAACATGTTTAAACA
TGGTACATTGAACTTAATTTTTGCAAGTTGATAAACTAGATTAATGTATG
ACTCATTTGTTTGTGCAAGTTGATAAACGTGATTAATATATGACTCATAT
GTTTGTGCAAAAATGGTGTCATCGTACAAACTCGCTTTACGAGTAGAATT
CTACTTGTAAAACACAATCGAGGGATGATGTCATTTGTAGAATGATGTCA
TTTGTTTTTTCAAAACCGAACTCGCTTTACGAGTAGAATTCTACTTGTAA
AACACAATCGAGGGATGATGTCATTTGTAGAATGATGTCATCGTACAAAC
TCGCTTTACGAGTAGAATTCTAGTAAAACAC

SEQ ID NO.21:TTGAAAATTTATTGCCTAATATTATTTTTGTCAGTTC
GTTGTCATTTATTAATTTGGATGATGTCCATTTGTTTTTAAAATTGAACT
GGCTTTACGAGTAGAATTCTACGCGTAAAACACAATCAAGTATGAGTCAT
AAGCTGATGTCATGTTTTGCACACGGCTCATAACCGAACTGGCTTTACGA
GTAGAATTCTACTTGTAACGCACGATCGAGTGGATGATGGTCATTTGTTT
TTCAAATCGAGATGATGTCATGTTTTGCACACGGGCTCATAAACTGCTTT
ACGAGTAGAATTCTACGTGTAACGCACGATCGATTGATGAGTCATTTGTT
TTGCAATATGATATCATACAATATGACTCATTTGTTTTTCAAAACCGAAC
TTGATTTACGGGTAGAATTCTACTCGTAAAGCACAATCAAAAAGATGATG
TCATTTGTTTTTCAAAACTGAACTCTCGGCTTTACGAGTAGAATTCTACG
TGTAAAACACAATCAAGAAATGATGTCATTTGTTATAAAAATAAAAGCTG
ATGTCATGTTTTGCACATGGCTCATAACTAAACTCGCTTTACGGGTAGAA
TTCTACGCGTAAAACATGATTGATAATTAAATAATTCATTTGCAAAGCTA
TACGTTAAATCAAACGGACGTTATGGAATTGTATAATATTAAATATGCAA
TTGATCCAACAAATAAAATTATAATAGAGCAAGTCGAC

Preferably, in some embodiments, the recombination homologous region of baculovirus is selected from hr1. It is believed that in some embodiments, hr1 is a repetitive sequence scattered throughout the baculovirus genome, and that hr1 is an origin of replication of baculovirus and also functions as an enhancer. Although the enhancement effect was not obvious in the early stage of infection, it became more significant in the late stage of infection. In some embodiments, the recombination homologous region of baculovirus (hr) includes hr2. In some embodiments, the recombination homologous region of baculovirus (hr) includes hr3. In some embodiments, the recombination homologous region of baculovirus (hr) includes hr4. In some embodiments, the recombination homologous region of baculovirus (hr) includes hr5.

In some embodiments, the nucleic acid construct further includes a baculovirus promoter. In some embodiments, the baculovirus promoter is selected from one or more (e.g., two, three, or four) of polyhedron promoter (pH), Gp64, p6.9, and p 10. It is believed that in some embodiments, hr is connected in cis to the baculovirus promoter to promote IE-mediated transactivation, and at the same time, IE will bind to hr1 in a dimer form to increase the expression of downstream proteins.

In some embodiments, the structure of the nucleic acid construct includes: IE gene expression cassette-Cap gene expression cassette-hr1-ITR-exogenous target gene expression cassette-ITR-Rep gene expression cassette. In some embodiments, the structure of the nucleic acid construct is: IE gene expression cassette-Cap gene expression cassette-ITR-exogenous target gene expression cassette-ITR-Rep gene expression cassette.

The gene expression cassette includes a gene and its promoter.

In a preferred embodiment, the structure of the nucleic acid construct includes: pA-IE1-pH-pA-Cap-p6.9p10-hr1-ITR-exogenous target gene and its promoter-pA-ITR-pH-Rep-pA. In a preferred embodiment, the structure of the nucleic acid construct is: pA-IE1-pH-pA-Cap-p6.9p10-hr1-ITR-exogenous target gene and its promoter-pA-ITR-pH-Rep-pA.

Specifically, in some embodiments, the nucleic acid construct is an AAV vector or a recombinant baculovirus vector. In some embodiments, the nucleic acid construct is an AAV vector. In some embodiments, the nucleic acid construct is a recombinant baculovirus vector. In some embodiments, the recombinant baculovirus vector is a recombinant baculovirus shuttle vector.

In some embodiments, the AAV vector or recombinant baculovirus vector further includes a vector backbone.

In some embodiments, the vector backbone can be selected from suitable vector backbones sold in the market, such as pFastBacdual, pFastBac1, pFastBacHTA, pFastBacHTB, pFastBacHTC, or the like.

In one embodiment, the vector backbone is pFastBacdual.

In some embodiments, the rAAV titer obtained by packaging the nucleic acid construct is at least 1×10¹² VG/mL (for example, at least 1.5×10¹², 2×10¹², 2.5×10¹², 3×10¹², 3.5×10^{12,} 4×10¹², 4.5×10¹², 5×10¹², 5.5×10¹², 6×10¹², 7×10¹², 7.5×10¹², 8×10¹², 8.5×10¹², 9×10¹², 9.5×10¹², 1×10¹³, 5×10¹³, or 1×10¹⁴ VG/mL), that is, the copy number of AAV genome contained in each mL of virus culture medium is at least 1×10¹² (for example, at least 1.5×10¹², 2×10¹², 2.5×10¹², 3×10¹², 3.5×10¹², 4×10¹², 4.5×10¹², 5×10¹², 5.5×10¹², 6×10¹², 7×10¹², 7.5×10¹², 8×10¹², 8.5×10¹², 9×10¹², 9.5×10¹², 1×10¹³, 5×10¹³, or 1×10¹⁴).

In some embodiments, the rAAV titer obtained by packaging the nucleic acid construct is at least 2.60E+12 VG/mL, that is, the copy number of the AAV genome contained in each mL of virus culture medium is at least 2.60E+12.

The yield of the rAAV is determined by qPCR method, and the detection steps are as follows: The standard used in quantitative PCR is pAAV-MCS plasmid linearized by single restriction enzyme digestion with PvuI-HF (NEB).

The primer sequences used in quantitative PCR are:
ITR Forward primer 5′-GGAACCCCTAGTGATGGAGTT-3′ (SEQ ID NO: 2)
ITR Reverse primer 5′-CGGCCTCAGTGAGCGA-3′ (SEQ ID NO: 3)

The running program of the quantitative PCR was: 95° C. for 60 s, (95° C. for 15 s, 60° C. for 30 s, 40 cycles). After plotting the standard curve according to the CT value obtained by quantitative PCR and the concentration of the standard, the titer of the sample was calculated.

The second aspect of the present invention provides a recombinant baculovirus, which is obtained by constructing any one of the nucleic acid constructs through the baculovirus system, or is obtained by constructing nucleic acid constructs by including any element of the said nucleic acid constructs through the baculovirus system.

The nucleic acid constructs comprising any element from any of said nucleic acid constructs mainly refer to: nucleic acid constructs comprising AAV elements, nucleic acid constructs comprising a polynucleotide encoding an IE protein, nucleic acid constructs comprising polynucleotides encoding recombination homologous region of baculovirus. The nucleic acid constructs comprising AAV elements can also be selected from nucleic acid constructs comprising a polynucleotide encoding Cap proteins, nucleic acid constructs comprising a polynucleotide encoding Rep proteins, and nucleic acid constructs comprising AAV cis-acting elements. The recombinant baculovirus can be constructed from the above nucleic acid constructs together through a baculovirus system.

The baculovirus system is selected from Bac-to-Bac system (derived from ThermoFisher/Invitrogen), flashBac/BacMagic system (derived from Mirus/EMD/OET/Nextgen), BaculoDirect system (derived from ThermoFisher/Invitrogen), and BacPAK6/Baculogold system (derived from BD Biosciences/Clonetech).

Specifically, the recombinant baculovirus is obtained by transforming competent cells with the nucleic acid construct, extracting bacmid, and transfecting Sf9 insect cells.

The competent cells can be selected from any applicable competent cells in the art, as long as the purpose of the present invention is not limited. For example, the competent cells can be DH10Bac.

The third aspect of the present invention provides an adeno-associated virus (AAV), which is obtained by packaging any one of the recombinant baculoviruses. The adeno-associated virus can be used to treat various diseases, such as hemophilia, spinal muscular atrophy, Duchenne muscular dystrophy, Parkinson’s disease, age-related macular degeneration, and the like.

The fourth aspect of the present invention provides a cell line, which is a cell line infected by any one of the recombinant baculoviruses.

In some embodiments, the cell line is an insect cell line, such as Sf9 cells, Sf21 cells, High5 cells.

The fifth aspect of the present invention provides an AAV vector system, which includes a baculovirus system and the nucleic acid construct.

The sixth aspect of the present invention provides the construction method of the nucleic acid construct, the construction method comprising integrating the AAV elements carrying the exogenous gene of interest and the polynucleotide encoding the IE protein into the backbone of the baculovirus vector.

The construction method also includes integrating the polynucleotide encoding the recombination homologous region of baculovirus into the backbone of the baculovirus vector.

In some embodiments, the AAV elements include a polynucleotide encoding Cap proteins, a polynucleotide encoding Rep proteins, and AAV cis-acting elements.

In some embodiments, the AAV cis-acting elements are selected from inverted terminal repeat sequences (ITR sequences).

Specifically, in some embodiments, the polynucleotide encoding the IE protein is linked to a strong promoter pH.

In some embodiments, the polynucleotide encoding the recombination homologous region of baculovirus is linked in cis to a baculovirus promoter. In some embodiments, the baculovirus promoter is p6.9 or p 10.

In some embodiments, the backbone of the baculovirus vector is selected from pFastBacdual, pFastBac1, pFastBacHTA, pFastBacHTB, pFastBacHTC, and etc.

In some embodiments, the recombination homologous region of baculovirus is selected from one or more (e.g., two, three, four, or five) of hr1, hr2, hr3, hr4, and hr5.

The seventh aspect of the present invention provides an AAV production method, the production method comprising the following steps: infecting insect cell lines with the recombinant baculovirus.

The production method can improve the yield of the AAV.

In some embodiments, the cell line is selected from an insect cell line. In one embodiment, the insect cell line is selected from Sf9 cells.

The eighth aspect of the present invention provides a method of treating diseases, said method comprising administering an effective amount of any one of the AAVs to patients.

In some embodiments, the diseases include, for example, hemophilia, spinal muscular atrophy, Duchenne muscular dystrophy, Parkinson’s disease, age-related macular degeneration, and the like.

The invention also includes any of the following numbered paragraphs:

1, A nucleic acid construct, characterized in that, the nucleic acid construct comprises: AAV elements, the polynucleotide encoding IE protein, and the AAV elements comprise the polynucleotide encoding Cap proteins, the polynucleotide encoding Rep proteins, and AAV cis-acting elements.

2. The nucleic acid construct according to paragraph 1, wherein the nucleic acid construct further comprises a polynucleotide encoding a recombination homologous region of baculovirus.

3. The nucleic acid construct according to paragraph 2, wherein the IE protein is selected from one or more encoded proteins of Acie0, Acie01, and Acie02, and/or, the recombination homologous region of baculovirus is selected from one or more of hr1, hr2, hr3, hr4, and hr5.

4. Any one of the nucleic acid constructs described in paragraphs 1-3, characterized in that the nucleic acid construct also includes the promoter of the IE protein-coding gene, and the promoter of the IE protein-coding gene is selected from one or more of Gp64, pH, p6.9, and p 10.

5. Any one of the nucleic acid constructs described in paragraphs 1-4, wherein the nucleic acid construct further comprises a baculovirus promoter connected to a recombination homologous region of baculovirus, and the baculovirus promoter is preferably one or more of pH, Gp64, p6.9, and p 10.

6. Any one of the nucleic acid constructs described in paragraphs 1-5, wherein the AAV cis-acting elements are selected from ITR sequences.

7. Any one of the nucleic acid constructs described in paragraphs 1-6, wherein the nucleic acid construct further comprises an exogenous gene of interest embedded in between AAV elements.

8. Any one of the nucleic acid constructs described in paragraphs 1-7, wherein the structure of the nucleic acid construct is: IE gene expression cassette-Cap gene expression cassette-ITR-exogenous target gene expression cassette-ITR- Rep gene expression cassette.

9. Any one of the nucleic acid constructs described in paragraphs 1-8, wherein the nucleotide sequence of the nucleic acid construct is as SEQ ID NO.1.

10. Any one of the nucleic acid constructs described in paragraphs 1-9, wherein the nucleic acid construct is an AAV vector or a recombinant baculovirus vector, and the recombinant baculovirus vector is preferably a recombinant baculovirus shuttle vector.

11. A recombinant baculovirus, characterized in that, the recombinant baculovirus is obtained by constructing the nucleic acid construct described in any one of paragraphs 1-10 through a baculovirus system, or by jointly constructing any of the nucleic acid constructs containing any element of the nucleic acid constructs described in paragraphs 1-10 through the baculovirus system.

12. An adeno-associated virus, characterized in that the adeno-associated virus is obtained after infection of cells with the recombinant baculovirus described in paragraph 11 and packaging.

13. A cell line, characterized in that the cell line is a cell line infected with the recombinant baculovirus described in paragraph 11.

14. An AAV vector system, characterized in that the AAV vector system comprises a baculovirus system and any one of the nucleic acid constructs described in paragraphs 1-10.

15. A method for constructing any one of the nucleic acid constructs described in paragraphs 1-10, characterized in that the construction method comprises integrating AAV elements carrying an exogenous gene of interest and the polynucleotide encoding an IE protein into the backbone of a baculovirus vector.

16. The construction method according to paragraph 15, wherein the construction method includes one or more of the following features:
1) The AAV elements include a polynucleotide encoding Cap proteins, a polynucleotide encoding Rep proteins, and AAV cis-acting elements, and the AAV cis-acting elements are preferably ITR sequences;
2) The IE protein is encoded by one or more of Acie0, Acie01, and Acie02 genes;
3) The backbone of the baculovirus vector is selected from one of pFastBacdual, pFastBac1, pFastBacHTA, pFastBacHTB, and pFastBacHTC;
4) The recombination homologous region of baculovirus is selected from one or more of hr1, hr2, hr3, hr4, and hr5;
5) Integrating the polynucleotide encoding the recombination homologous region of baculovirus into the backbone of the baculovirus vector.

17. A production method of adeno-associated virus, characterized in that the production method comprises the following steps: infecting insect cell lines with the recombinant baculovirus described in paragraph 11.

Embodiments of the present invention are described below through specific examples, and those skilled in the art can easily understand other advantages and effects of the present invention from the content disclosed in this specification. The present invention can also be implemented or applied through other different specific implementation modes, and various modifications or changes can be made to the details in this specification based on different viewpoints and applications without departing from the spirit of the present invention.

Before further describing the specific embodiments of the present invention, it should be understood that the protection scope of the present invention is not limited to the following specific embodiments; it should also be understood that the terms used in the examples of the present invention are to describe specific embodiments, not intended to limit the protection scope of the present invention; in the description and claims of the present invention, unless the context clearly indicates otherwise, the singular forms “a”, “an” and “the” include plural forms.

When the examples give numerical ranges, it should be understood that, unless otherwise stated in the present invention, the two endpoints of each numerical range and any value between the two endpoints can be selected. Unless defined otherwise, all technical and scientific terms used herein have the same meaning as commonly understood by one of ordinary skill in the art. In addition to the specific methods, equipment, and materials used in the embodiments, according to those skilled in the art’s grasp of the prior art and the description of the present invention, any methods, equipment, and materials of the prior art similar or equivalent to the practice of the present invention can also be used.

The following examples take EGFP as an exogenous target gene as an example.

### Example 1. Construction of Recombinant Baculovirus Shuttle Vector and Acquisition of Recombinant Baculovirus

Identify related genes: Both Cap and Rep protein-coding genes are derived from the AAV2 genome (GenBank: AF043303.1), according to the ribosome leak scanning mechanism reference (Smith RH, Levy JR, Kotin RM: A simplified baculovirus-AAV expression vector system coupled with one-step affinity purification yields high-titer rAAV stocks from insect cells. Mol Ther 2009, 17(11):1888-1896) point mutations of bases were generated; Acie01 (GenBank: NC_001623.1), recombination homologous region of baculovirus hr1 (GenBank: M14313.1).

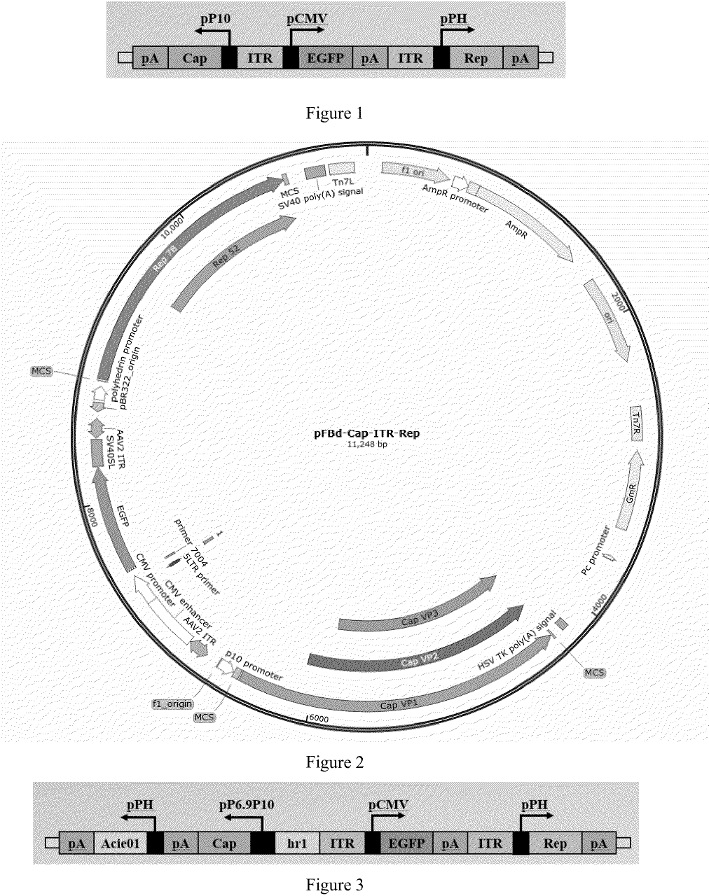

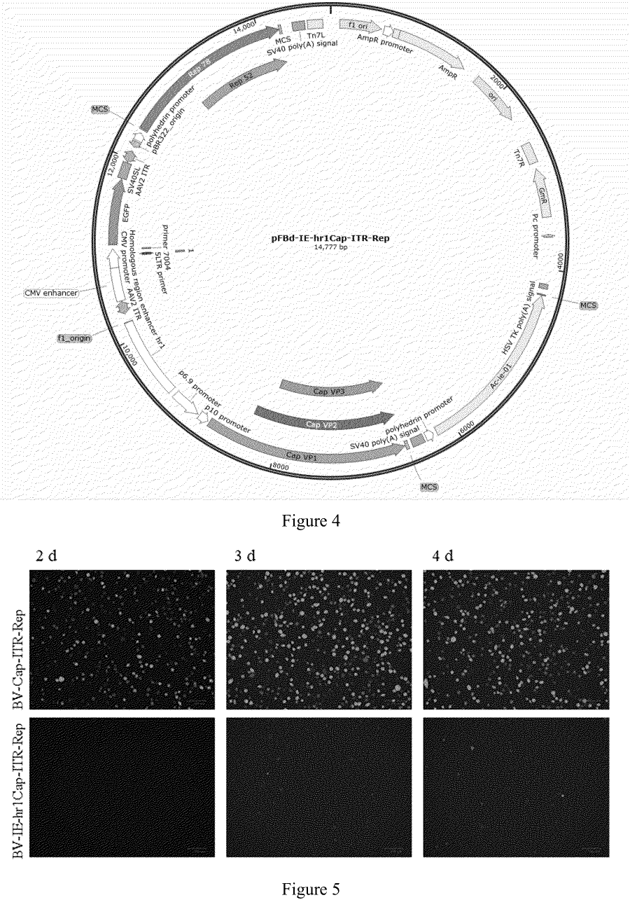

Entrust the gene company to synthesize the corresponding sequences: Cap gene sequence (nucleotide sequence SEQ ID NO: 4), Rep gene sequence (nucleotide sequence SEQ ID NO: 5), hr1 and p6.9, p10 promoter combination (hr1p6.9p10, nucleotide sequence of SEQ ID NO: 6), pH promoter and Acie01 combination (pH-Acie01, nucleotide sequence of SEQ ID NO: 7). The above sequences and the ITR-CMV-EGFP sequence in the pAAV-EGFP vector (modified by Kanglin Biotech (Hangzhou) Co., Ltd.) were cloned into pFastBacdual (Invitrogen) using the homologous recombination method well known in the art, and two recombinant baculovirus shuttle vectors pFBd-Cap-ITR-Rep (FIG. 1 and FIG. 2, nucleotide sequence SEQ ID NO: 8) and pFBd-IE-hr1Cap-ITR-Rep (FIG. 3 and FIG. 4, nucleotide sequence SEQ ID NO: 9) were obtained after identification by sequencing.

The sequences of SEQ ID NO.4-9 are as follows:

SEQ ID NO: 4:ACGGCTGCCGACGGTTATCTACCCGATTGGCTCGAGG
ACACTCTCTCTGAAGGAATAAGACAGTGGTGGAAGCTCAAACCCGGCCCA
CCACCACCAAAGCCCGCAGAGCGGCATAAGGACGACAGCAGGGGCCTTGT
GCTTCCCGGGTACAAGTACCTCGGACCCTTCAACGGACTGGACAAGGGAG
AGCCGGTGAACGAGGCAGACGCCGCTGCCCTCGAGCACGACAAAGCCTAC
GACCGGCAGCTCGACAGCGGAGACAACCCTTACCTCAAGTACAACCACGC
CGACGCGGAGTTCCAGGAGCGCCTTAAAGAAGATACGTCTTTTGGGGGCA
ACCTCGGACGAGCAGTCTTCCAGGCGAAAAAGAGGGTTCTTGAACCTCTG
GGCCTGGTTGAGGAACCTGTTAAGACGGCTCCGGGAAAAAAGAGGCCGGT
AGAGCACTCTCCTGTGGAGCCAGACTCCTCCTCGGGAACCGGAAAGGCGG
GCCAGCAGCCTGCAAGAAAAAGATTGAATTTTGGTCAGACTGGAGACGCA
GACTCAGTACCTGACCCCCAGCCTCTCGGACAGCCACCAGCAGCCCCCTC
TGGTCTGGGAACTAATACGATGGCTACAGGCAGTGGCGCACCAATGGCAG
ACAATAACGAGGGCGCCGACGGAGTGGGTAATTCCTCGGGAAATTGGCAT
TGCGATTCCACATGGATGGGCGACAGAGTCATCACCACCAGCACCCGAAC
CTGGGCCCTGCCCACCTACAACAACCACCTCTACAAACAAATTTCCAGCC
AATCAGGAGCCTCGAACGACAATCACTACTTTGGCTACAGCACCCCTTGG
GGGTATTTTGACTTCAACAGATTCCACTGCCACTTTTCACCACGTGACTG
GCAAAGACTCATCAACAACAACTGGGGATTCCGACCCAAGAGACTCAACT
TCAAGCTCTTTAACATTCAAGTCAAAGAGGTCACGCAGAATGACGGTACG
ACGACGATTGCCAATAACCTTACCAGCACGGTTCAGGTGTTTACTGACTC
GGAGTACCAGCTCCCGTACGTCCTCGGCTCGGCGCATCAAGGATGCCTCC
CGCCGTTCCCAGCAGACGTCTTCATGGTGCCACAGTATGGATACCTCACC
CTGAACAACGGGAGTCAGGCAGTAGGACGCTCTTCATTTTACTGCCTGGA
GTACTTTCCTTCTCAGATGCTGCGTACCGGAAACAACTTTACCTTCAGCT
ACACTTTTGAGGACGTTCCTTTCCACAGCAGCTACGCTCACAGCCAGAGT
CTGGACCGTCTCATGAATCCTCTCATCGACCAGTACCTGTATTACTTGAG
CAGAACAAACACTCCAAGTGGAACCACCACGCAGTCAAGGCTTCAGTTTT
CTCAGGCCGGAGCGAGTGACATTCGGGACCAGTCTAGGAACTGGCTTCCT
GGACCCTGTTACCGCCAGCAGCGAGTATCAAAGACATCTGCGGATAACAA
CAACAGTGAATACTCGTGGACTGGAGCTACCAAGTACCACCTCAATGGCA
GAGACTCTCTGGTGAATCCGGGCCCGGCGATGGCAAGCCACAAGGACGAT
GAAGAAAAGTTTTTTCCTCAGAGCGGGGTTCTCATCTTTGGGAAGCAAGG
CTCAGAGAAAACAAATGTGGACATTGAAAAGGTCATGATTACAGACGAAG
AGGAAATCAGGACAACCAATCCCGTGGCTACGGAGCAGTATGGTTCTGTA
TCTACCAACCTCCAGAGAGGCAACAGACAAGCAGCTACCGCAGATGTCAA
CACACAAGGCGTTCTTCCAGGCATGGTCTGGCAGGACAGAGATGTGTACC
TTCAGGGGCCCATCTGGGCAAAGATTCCACACACGGACGGACATTTTCAC
CCCTCTCCCCTCATGGGTGGATTCGGACTTAAACACCCTCCTCCACAGAT
TCTCATCAAGAACACCCCGGTACCTGCGAATCCTTCGACCACCTTCAGTG
CGGCAAAGTTTGCTTCCTTCATCACACAGTACTCCACGGGACAGGTCAGC
GTGGAGATCGAGTGGGAGCTGCAGAAGGAAAACAGCAAACGCTGGAATCC
CGAAATTCAGTACACTTCCAACTACAACAAGTCTGTTAATGTGGACTTTA
CTGTGGACACTAATGGCGTGTATTCAGAGCCTCGCCCCATTGGCACCAGA
TACCTGACTCGTAATCTGTAA

SEQ ID NO: 5:CTGGCGGGGTTTTACGAGATTGTGATTAAGGTCCCCA
GCGACCTTGACGAGCATCTGCCCGGCATTTCTGACAGCTTTGTGAACTGG
GTGGCCGAGAAGGAGTGGGAGTTGCCGCCAGATTCTGACTTGGATCTGAA
TCTGATTGAGCAGGCACCCCTGACCGTGGCCGAGAAGCTGCAGCGCGACT
TTCTGACGGAGTGGCGCCGTGTGAGTAAGGCCCCGGAGGCCCTTTTCTTT
GTGCAATTTGAGAAGGGAGAGAGCTACTTCCACTTACACGTGCTCGTGGA
AACCACCGGGGTGAAATCCTTAGTTTTGGGACGTTTCCTGAGTCAGATTC
GCGAAAAACTGATTCAGAGAATTTACCGCGGGATCGAGCCGACTTTGCCA
AACTGGTTCGCGGTCACAAAGACCAGAAACGGCGCCGGAGGCGGGAACAA
GGTGGTGGACGAGTGCTACATCCCCAATTACTTGCTCCCCAAAACCCAGC
CTGAGCTCCAGTGGGCGTGGACTAATTTAGAACAGTATTTAAGCGCCTGT
TTGAATCTCACGGAGCGTAAACGGTTGGTGGCGCAGCATCTGACGCACGT
GTCGCAGACGCAGGAGCAGAACAAAGAGAATCAGAATCCCAATTCTGACG
CGCCGGTGATCAGATCAAAAACTTCAGCCAGGTACATGGAGCTGGTCGGG
TGGCTCGTGGACAAGGGGATTACCTCGGAGAAGCAGTGGATACAGGAGGA
CCAGGCCTCATACATCTCCTTCAATGCGGCCTCCAACTCGCGGTCCCAAA
TCAAGGCTGCCTTGGACAATGCGGGAAAGATTATGAGCCTGACTAAAACC
GCCCCCGACTACCTGGTGGGCCAGCAGCCCGTGGAGGACATTTCCAGCAA
TCGGATTTATAAAATTTTGGAACTAAACGGGTACGATCCCCAATATGCGG
CTTCCGTCTTTCTGGGATGGGCCACGAAAAAGTTCGGCAAGAGGAACACC
ATCTGGCTGTTTGGGCCTGCAACTACCGGGAAGACCAACATCGCGGAGGC
CATAGCCCACACTGTGCCCTTCTACGGGTGCGTAAACTGGACCAATGAGA
ACTTTCCCTTCAACGACTGTGTGGACAAGATGGTGATCTGGTGGGAGGAG
GGGAAGATGACCGCCAAGGTCGTGGAGTCGGCCAAAGCCATTCTCGGAGG
AAGCAAGGTGCGCGTGGACCAGAAATGCAAGTCCTCGGCCCAGATAGACC
CGACTCCCGTGATCGTCACCTCCAACACCAACATGTGCGCCGTGATTGAC
GGGAACTCAACGACCTTCGAACACCAGCAGCCGTTGCAAGACCGGATGTT
CAAATTTGAACTCACCCGCCGTCTGGATCATGACTTTGGGAAGGTCACCA
AGCAGGAAGTCAAAGACTTTTTCCGGTGGGCAAAGGATCACGTGGTTGAG
GTGGAGCATGAATTCTACGTCAAAAAGGGTGGAGCCAAGAAAAGACCCGC
CCCCAGTGACGCAGATATAAGTGAGCCCAAACGGGTGCGCGAGTCAGTTG
CGCAGCCATCGACGTCAGACGCGGAAGCATCGATCAACTACGCAGACAGG
TACCAAAACAAATGTTCTCGTCACGTGGGCATGAATCTGATGCTGTTTCC
CTGCAGACAATGCGAGAGAATGAATCAGAATTCAAATATCTGCTTCACTC
ACGGACAGAAAGACTGTTTAGAGTGCTTTCCCGTGTCAGAATCTCAACCC
GTTTCTGTCGTCAAAAAGGCGTATCAGAAACTGTGCTACATTCATCATAT
CATGGGAAAGGTGCCAGACGCTTGCACTGCCTGCGATCTGGTCAATGTGG
ATTTGGATGACTGCATCTTTGAACAATAA

SEQ ID NO: 6:ATCGATGTTGACCCCAACAAAAGATTTATAATTAATC
ATAATCACGAACAACAACAAGTCAATGAAACAAATAAACAAGTTGTCGAT
AAAACATTCATAAATGACACAGCAACATACAATTCTTGCATAATAAAAAT
TTAAATGACATCATATTTGAGAATAACAAATGACATTATCCCTCGATTGT
GTTTTACAAGTAGAATTCTACCCGTAAAGCGAGTTTAGTTTTGAAAAACA
AATGACATCATTTGTATAATGACATCATCCCCTGATTGTGTTTTACAAGT
AGAATTCTATCCGTAAAGCGAGTTCAGTTTTGAAAACAAATGAGTCATAC
CTAAACACGTTAATAATCTTCTGATATCAGCTTATGACTCAAGTTATGAG
CCGTGTGCAAAACATGAGATAAGTTTATGACATCATCCACTGATCGTGCG
TTACAAGTAGAATTCTACTCGTAAAGCCAGTTCGGTTATGAGCCGTGTGC
AAAACATGACATCAGCTTATGACTCATACTTGATTGTGTTTTACGCGTAG
AATTCTACTCGTAAAGCGAGTTCGGTTATGAGCCGTGTGCAAAACATGAC
ATCAGCTTATGAGTCATAATTAATCGTGCGTTACAAGTAGAATTCTACTC
GTAAAGCGAGTTGAAGGATCATATTTAGTTGCGTTTATGAGATAAGATTG
AAAGCACGTGTAAAATGTTTCCCGCGCGTTGGCACAACTATTTACAATGC
GGCCAAGTTATAAAAGATTCTAATCTGATATGTTTTAAAACACCTTTGCG
GCCCGAGTTGTTTGCGTACGTGACTAGCGAAGAAGATGTGTGGACCGCAG
AACAGATAGTAAAACAAAACCCTAGTATTGGAGCAATAATCGATGACGTC
GCTGACGTAGCGCTTTGAATTCCGCGCGCTTCGGACCGGGATCGTGACCA
AATTCCGTTTTGCGACGATGCAGAGTTTTTGAACAGGCTGCTCAAACACA
TAGATCCGTACCCGCTCAGTCGGATGTATTACAATGCAGCCAATACCATG
TTTTACACGACTATGGAAAACTATGCCGTGTCCAATTGCAAGTTCAACAT
TGAGGATTACAATAACATATTTAAGGTGATGGAAAATATTAGGAAACACA
GCAACAAAAATTCAAACGACCAAGACGAGTTAAACATATATTTGGGAGTT
CAGTCGTCGAATGCAAAGCGTAAAAAATATTAATAAGGTAAAAATTACAG
CTACATAAATTACACAATTTAAACTGCAGTCTGGAGATACGGACCTTTAA
TTCAACCCAACACAATATATTATAGTTAAATAAGAATTATTATCAAATCA
TTTGTATATTAATTAAAATACTATACTGTAAATTACATTTTATTTACAAT
C

SEQ ID NO: 7:AACTTGTTTATTGCAGCTTATAATGGTTACAAATAAA
GCAATAGCATCACAAATTTCACAAATAAAGCATTTTTTTCACTGCATTCT
AGTTGTGGTTTGTCCAAACTCATCAATGTATCTTATCATGTCTGGATCTG
ATCACTGCTTGAGCCTAGGAGATCCGATCATGGAGATAATTAAAATGATA
ACCATCTCGCAAATAAATAAGTATTTTACTGTTTTCGTAACAGTTTTGTA
ATAAAAAAACCTATAAATATTCCGGATTATTCATACCGTCCCACCATCGG
GCGCATGATCCGTACATCCAGCCACGTCCTGAACGTCCAAGAAAACATCA
TGACTTCCAACTGTGCTTCCAGCCCCTACTCCTGTGAGGCCACTTCAGCC
TGCGCTGAGGCCCAGCAACTGCAGGTGGACACAGGTGGCGATAAGATCGT
GAACAACCAGGTCACCATGACTCAAATCAACTTCAACGCTTCCTACACCT
CTGCCAGCACTCCCTCTCGTGCTAGCTTCGACAACTCATACTCGGAGTTC
TGCGACAAGCAACCTAACGATTACTTGTCTTACTACAACCACCCAACCCC
GGACGGAGCTGATACTGTCATCTCCGACTCTGAAACCGCTGCCGCTAGCA
ACTTCCTCGCCTCAGTTAACTCGCTCACTGACAACGATTTGGTGGAGTGT
CTGCTCAAGACCACTGACAACCTGGAGGAAGCTGTGTCCTCTGCCTACTA
CAGCGAGTCACTCGAACAGCCAGTGGTCGAACAACCCTCTCCTAGCTCAG
CTTACCACGCCGAGTCCTTCGAACACTCTGCTGGTGTCAACCAGCCGTCG
GCCACAGGCACCAAGAGGAAGTTGGACGAGTACCTGGATAACTCCCAGGG
AGTTGTGGGTCAATTCAACAAGATCAAGTTGAGACCTAAGTACAAGAAGA
GCACCATCCAGTCATGCGCTACACTGGAACAAACCATCAACCACAACACT
AACATCTGTACAGTGGCTTCCACCCAGGAGATCACTCACTACTTCACAAA
CGACTTCGCCCCCTACCTGATGAGGTTCGACGATAACGACTACAACTCGA
ACAGATTCTCCGATCACATGTCTGAAACCGGTTACTACATGTTCGTCGTT
AAGAAGTCCGAGGTGAAGCCTTTCGAAATCATCTTCGCCAAGTACGTCTC
TAACGTGGTCTACGAGTACACAAACAACTACTACATGGTTGACAACCGTG
TGTTCGTTGTGACCTTCGATAAGATCCGCTTCATGATCAGCTACAACCTG
GTTAAGGAGACTGGCATCGAAATCCCACACTCACAGGACGTCTGCAACGA
TGAGACCGCCGCTCAAAACTGCAAGAAGTGTCACTTCGTGGACGTCCACC
ACACATTCAAGGCCGCTCTGACCTCCTACTTCAACCTCGATATGTACTAC
GCTCAGACAACCTTCGTGACCTTGCTGCAATCACTCGGCGAGCGTAAGTG
TGGATTCCTCTTGTCGAAGTTGTACGAGATGTACCAGGACAAGAACCTCT
TCACTTTGCCCATCATGCTGAGCCGCAAGGAATCAAACGAGATCGAAACC
GCCTCTAACAACTTCTTCGTCTCGCCATACGTTTCCCAGATCCTCAAGTA
CTCGGAGTCCGTCCAATTCCCGGACAACCCTCCCAACAAGTACGTCGTTG
ATAACCTGAACCTCATCGTGAACAAGAAGAGCACTCTGACATACAAGTAC
TCGTCCGTCGCTAACCTGCTCTTCAACAACTACAAGTACCACGACAACAT
CGCTTCTAACAACAACGCCGAGAACCTCAAGAAGGTCAAGAAGGAAGACG
GAAGCATGCACATCGTTGAGCAGTACTTGACTCAAAACGTCGATAACGTT
AAGGGTCACAACTTCATCGTGTTGTCCTTCAAGAACGAGGAAAGGCTGAC
CATCGCTAAGAAGAACAAGGAGTTCTACTGGATCTCTGGCGAAATCAAGG
ACGTTGATGTGAGCCAGGTCATCCAAAAGTACAACAGATTCAAGCACCAC
ATGTTCGTGATCGGCAAGGTCAACCGTCGCGAGTCAACTACACTGCACAA
CAACTTGCTGAAGCTCTTGGCCTTGATCCTGCAGGGACTGGTGCCACTCT
CCGACGCCATCACATTCGCCGAGCAAAAGCTCAACTGCAAGTACAAGAAG
TTCGAGTTCAACTAA

SEQ ID NO: 8:TTCTCTGTCACAGAATGAAAATTTTTCTGTCATCTCT
TCGTTATTAATGTTTGTAATTGACTGAATATCAACGCTTATTTGCAGCCT
GAATGGCGAATGGGACGCGCCCTGTAGCGGCGCATTAAGCGCGGCGGGTG
TGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGT
GGGCCATCGCCCTGATAGACGGTTTTTCGCCCTTTGACGTTGGAGTCCAC
GTTCTTTAATAGTGGACTCTTGTTCCAAACTGGAACAACACTCAACCCTA
TCTCGGTCTATTCTTTTGATTTATAAGGGATTTTGCCGATTTCGGCCTAT
TGGTTAAAAAATGAGCTGATTTAACAAAAATTTAACGCGAATTTTAACAA
AATATTAACGTTTACAATTTCAGGTGGCACTTTTCGGGGAAATGTGCGCG
GAACCCCTATTTGTTTATTTTTCTAAATACATTCAAATATGTATCCGCTC
ATGAGACAATAACCCTGATAAATGCTTCAATAATATTGAAAAAGGAAGAG
TATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAA
CAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGAC
GCCGGGCAAGAGCAACTCGGTCGCCGCATACACTATTCTCAGAATGACTT
GGTTGAGTACTCACCAGTCACAGAAAAGCATCTTACGGATGGCATGACAG
TAAGAGAATTATGCAGTGCTGCCATAACCATGAGTGATAACACTGCGGCC
AACTTACTTCTGACAACGATCGGAGGACCGAAGGAGCTAACCGCTTTTTT
GCACAACATGGGGGATCATGTAACTCGCCTTGATCGTTGGGAACCGGAGC
TGAATGAAGCCATACCAAACGACGAGCGTGACACCACGATGCCTGTAGCA
ATGGCAACAACGTTGCGCAAACTATTAACTGGCGAACTACTTACTCTAGC
TTCCCGGCAACAATTAATAGACTGGATGGAGGCGGATAAAGTTGCAGGAC
CACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAA
CTATGGATGAACGAAATAGACAGATCGCTGAGATAGGTGCCTCACTGATT
AAGCATTGGTAACTGTCAGACCAAGTTTACTCATATATACTTTAGATTGA
TTTAAAACTTCATTTTTAATTTAAAAGGATCTAGGTGAAGATCCTTTTTG
ATAATCTCATGACCAAAATCCCTTAACGTGAGTTTTCGTTCCACTGAGCG
TCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTT
AGGCCACCACTTCAAGAACTCTGTAGCACCGCCTACATACCTCGCTCTGC
TAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTG
AACGGGGGGTTCGTGCACACAGCCCAGCTTGGAGCGAACGACCTACACCG
AACTGAGATACCTACAGCGTGAGCATTGAGAAAGCGCCACGCTTCCCGAA
GGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCC
CTGATTCTGTGGATAACCGTATTACCGCCTTTGAGTGAGCTGATACCGCT
CGCCGCAGCCGAACGACCGAGCGCAGCGAGTCAGTGAGCGAGGAAGCGGA
AGAGCGCCTGATGCGGTATTTTCTCCTTACGCATCTGTGCGGTATTTCAC
ACCGCAGACCAGCCGCGTAACCTGGCAAAATCGGTTACGGTTGAGTAATA
AATGGATGCCCTGCGTAAGCGGGTGTGGGCGGACAATAAAGTCTTAAACT
GAACAAAATAGATCTAAACTATGACAATAAAGTCTTAAACTAGACAGAAT
AGTTGTAAACTGAAATCAGTCCAGTTATGCTGTGAAAAAGCATACTGGAC
TTTTGTTATGGCTAAAGCAAACTCTTCATTTTCTGAAGTGCAAATTGCCC
GTCGTATTAAAGAGGGGCGTGGCCAAGGGCATGGTAAAGACTATATTCGC
GGCGTTGTGACAATTTACCGAACAACTCCGCGGCCGGGAAGCCGATCTCG
GCTTGAACGAATTGTTAGGTGGCGGTACTTGGGTCGATATCAAAGTGCAT
CACTTCTTCCCGTATGCCCAACTTTGTATAGAGAGCCACTGCGGGATCGT
CACCGTAATCTGCTTGCACGTAGATCACATAAGCACCAAGCGCGTTGGCC
TCATGCTTGAGGAGATTGATGAGCGCGGTGGCAATGCCCTGCCTCCGGTG
CTCGCCGGAGACTGCGAGATCATAGATATAGATCTCACTACGCGGCTGCT
CAAACCTGGGCAGAACGTAAGCCGCGAGAGCGCCAACAACCGCTTCTTGG
TCGAAGGCAGCAAGCGCGATGAATGTCTTACTACGGAGCAAGTTCCCGAG
GTAATCGGAGTCCGGCTGATGTTGGGAGTAGGTGGCTACGTCTCCGAACT
CACGACCGAAAAGATCAAGAGCAGCCCGCATGGATTTGACTTGGTCAGGG
CCGAGCCTACATGTGCGAATGATGCCCATACTTGAGCCACCTAACTTTGT
TTTAGGGCGACTGCCCTGCTGCGTAACATCGTTGCTGCTGCGTAACATCG
TTGCTGCTCCATAACATCAAACATCGACCCACGGCGTAACGCGCTTGCTG
CTTGGATGCCCGAGGCATAGACTGTACAAAAAAACAGTCATAACAAGCCA
TGAAAACCGCCACTGCGCCGTTACCACCGCTGCGTTCGGTCAAGGTTCTG
GACCAGTTGCGTGAGCGCATACGCTACTTGCATTACAGTTTACGAACCGA
ACAGGCTTATGTCAACTGGGTTCGTGCCTTCATCCGTTTCCACGGTGTGC
GTCACCCGGCAACCTTGGGCAGCAGCGAAGTCGAGGCATTTCTGTCCTGG
CTGGCGAACGAGCGCAAGGTTTCGGTCTCCACGCATCGTCAGGCATTGGC
GGCCTTGCTGTTCTTCTACGGCAAGGTGCTGTGCACGGATCTGCCCTGGC
TTCAGGAGATCGGTAGACCTCGGCCGTCGCGGCGCTTGCCGGTGGTGCTG
ACCCCGGATGAAGTGGTTCGCATCCTCGGTTTTCTGGAAGGCGAGCATCG
TTTGTTCGCCCAGGACTCTAGCTATAGTTCTAGTGGTTGGCCTACGTACC
CGTAGTGGCTATGGCAGGGCTTGCCGCCCCGACGTTGGCTGCGAGCCCTG
GGCCTTCACCCGAACTTGGGGGTTGGGGTGGGGAAAAGGAAGAAACGCGG
GCGTATTGGTCCCAATGGGGTCTCGGTGGGGTATCGACAGAGTGCCAGCC
CTGGGACCGAACCCCGCGTTTATGAACAAACGACCCAACACCCGTGCGTT
TTATTCTGTCTTTTTATTGCCGTCATAGCGCGGGTTCCTTCCGGTATTGT
CTCCTTCCGTGTTTCAGTTAGCCTCCCCCATCTCCCGGTACCGCATGCTT
ACAGATTACGAGTCAGGTATCTGGTGCCAATGGGGCGAGGCTCTGAATAC
ACGCCATTAGTGTCCACAGTAAAGTCCACATTAACAGACTTGTTGTAGTT
GGAAGTGTACTGAATTTCGGGATTCCAGCGTTTGCTGTTTTCCTTCTGCA
GCTCCCACTCGATCTCCACGCTGACCTGTCCCGTGGAGTACTGTGTGATG
AAGGAAGCAAACTTTGCCGCACTGAAGGTGGTCGAAGGATTCGCAGGTAC
CGGGGTGTTCTTGATGAGAATCTGTGGAGGAGGGTGTTTAAGTCCGAATC
CACCCATGAGGGGAGAGGGGTGAAAATGTCCGTCCGTGTGTGGAATCTTT
GCCCAGATGGGCCCCTGAAGGTACACATCTCTGTCCTGCCAGACCATGCC
TGGAAGAACGCCTTGTGTGTTGACATCTGCGGTAGCTGCTTGTCTGTTGC
CTCTCTGGAGGTTGGTAGATACAGAACCATACTGCTCCGTAGCCACGGGA
TTGGTTGTCCTGATTTCCTCTTCGTCTGTAATCATGACCTTTTCAATGTC
CACATTTGTTTTCTCTGAGCCTTGCTTCCCAAAGATGAGAACCCCGCTCT
GAGGAAAAAACTTTTCTTCATCGTCCTTGTGGCTTGCCATCGCCGGGCCC
GGATTCACCAGAGAGTCTCTGCCATTGAGGTGGTACTTGGTAGCTCCAGT
CCACGAGTATTCACTGTTGTTGTTATCCGCAGATGTCTTTGATACTCGCT
GCTGGCGGTAACAGGGTCCAGGAAGCCAGTTCCTAGACTGGTCCCGAATG
TCACTCGCTCCGGCCTGAGAAAACTGAAGCCTTGACTGCGTGGTGGTTCC
ACTTGGAGTGTTTGTTCTGCTCAAGTAATACAGGTACTGGTCGATGAGAG
GATTCATGAGACGGTCCAGACTCTGGCTGTGAGCGTAGCTGCTGTGGAAA
GGAACGTCCTCAAAAGTGTAGCTGAAGGTAAAGTTGTTTCCGGTACGCAG
CATCTGAGAAGGAAAGTACTCCAGGCAGTAAAATGAAGAGCGTCCTACTG
CCTGACTCCCGTTGTTCAGGGTGAGGTATCCATACTGTGGCACCATGAAG
ACGTCTGCTGGGAACGGCGGGAGGCATCCTTGATGCGCCGAGCCGAGGAC
GTACGGGAGCTGGTACTCCGAGTCAGTAAACACCTGAACCGTGCTGGTAA
GGTTATTGGCAATCGTCGTCGTACCGTCATTCTGCGTGACCTCTTTGACT
TGAATGTTAAAGAGCTTGAAGTTGAGTCTCTTGGGTCGGAATCCCCAGTT
GTTGTTGATGAGTCTTTGCCAGTCACGTGGTGAAAAGTGGCAGTGGAATC
TGTTGAAGTCAAAATACCCCCAAGGGGTGCTGTAGCCAAAGTAGTGATTG
TCGTTCGAGGCTCCTGATTGGCTGGAAATTTGTTTGTAGAGGTGGTTGTT
GTAGGTGGGCAGGGCCCAGGTTCGGGTGCTGGTGGTGATGACTCTGTCGC
CCATCCATGTGGAATCGCAATGCCAATTTCCCGAGGAATTACCCACTCCG
TCGGCGCCCTCGTTATTGTCTGCCATTGGTGCGCCACTGCCTGTAGCCAT
CGTATTAGTTCCCAGACCAGAGGGGGCTGCTGGTGGCTGTCCGAGAGGCT
GGGGGTCAGGTACTGAGTCTGCGTCTCCAGTCTGACCAAAATTCAATCTT
TTTCTTGCAGGCTGCTGGCCCGCCTTTCCGGTTCCCGAGGAGGAGTCTGG
CTCCACAGGAGAGTGCTCTACCGGCCTCTTTTTTCCCGGAGCCGTCTTAA
CAGGTTCCTCAACCAGGCCCAGAGGTTCAAGAACCCTCTTTTTCGCCTGG
AAGACTGCTCGTCCGAGGTTGCCCCCAAAAGACGTATCTTCTTTAAGGCG
CTCCTGGAACTCCGCGTCGGCGTGGTTGTACTTGAGGTAAGGGTTGTCTC
CGCTGTCGAGCTGCCGGTCGTAGGCTTTGTCGTGCTCGAGGGCAGCGGCG
TCTGCCTCGTTCACCGGCTCTCCCTTGTCCAGTCCGTTGAAGGGTCCGAG
GTACTTGTACCCGGGAAGCACAAGGCCCCTGCTGTCGTCCTTATGCCGCT
CTGCGGGCTTTGGTGGTGGTGGGCCGGGTTTGAGCTTCCACCACTGTCTT
ATTCCTTCAGAGAGAGTGTCCTCGAGCCAATCGGGTAGATAACCGTCGGC
AGCCGTGGCGGCGCTAGCACCATGGCTCGAGATCCCGGGTGATCAAGTCT
TCGTCGAGTGATTGTAAATAAAATGTAATTTACAGTATAGTATTTTAATT
AATATACAAATGATTTGATAATAATTCTTATTTAACTATAATATATTGTG
TTGGGTTGAATTAAAGGTCCGTATACGCCGCTACAGGGCGCGTACTATGG
TTGCTTTGACGTATGCGGTGTGAAATACCGCACAGATGCGTAAGGAGAAA
ATACCGCATCAGGCGCCCCTGCAGGCAGCTGCGCGCTCGCTCGCTCACTG
AGGCCGCCCGGGCAAAGCCCGGGCGTCGGGCGACCTTTGGTCGCCCGGCC
TCAGTGAGCGAGCGAGCGCGCAGAGAGGGAGTGGCCAACTCCATCACTAG
GGGTTCCTACGCGTTAGTTATTAATAGTAATCAATTACGGGGTCATTAGT
TCATAGCCCATATATGGAGTTCCGCGTTACATAACTTACGGTAAATGGCC
CGCCTGGCTGACCGCCCAACGACCCCCGCCCATTGACGTCAATAATGACG
TATGTTCCCATAGTAACGCCAATAGGGACTTTCCATTGACGTCAATGGGT
GGAGTATTTACGGTAAACTGCCCACTTGGCAGTACATCAAGTGTATCATA
TGCCAAGTACGCCCCCTATTGACGTCAATGACGGTAAATGGCCCGCCTGG
CATTATGCCCAGTACATGACCTTATGGGACTTTCCTACTTGGCAGTACAT
CTACGTATTAGTCATCGCTATTACCATGGTGATGCGGTTTTGGCAGTACA
TCAATGGGCGTGGATAGCGGTTTGACTCACGGGGATTTCCAAGTCTCCAC
CCCATTGACGTCAATGGGAGTTTGTTTTGGCACCAAAATCAACGGGACTT
TCCAAAATGTCGTAACAACTCCGCCCCATTGACGCAAATGGGCGGTAGGC
GTGTACGGTGGGAGGTCTATATAAGCAGAGCTGGTTTAGTGAACCGTCAG
ATCCGCTAGCGCTACCGGTCGCCACCATGGTGAGCAAGGGCGAGGAGCTG
TTCACCGGGGTGGTGCCCATCCTGGTCGAGCTGGACGGCGACGTAAACGG
CCACAAGTTCAGCGTGTCCGGCGAGGGCGAGGGCGATGCCACCTACGGCA
AGCTGACCCTGAAGTTCATCTGCACCACCGGCAAGCTGCCCGTGCCCTGG
CCCACCCTCGTGACCACCCTGACCTACGGCGTGCAGTGCTTCAGCCGCTA
CCCCGACCACATGAAGCAGCACGACTTCTTCAAGTCCGCCATGCCCGAAG
GCTACGTCCAGGAGCGCACCATCTTCTTCAAGGACGACGGCAACTACAAG
ACCCGCGCCGAGGTGAAGTTCGAGGGCGACACCCTGGTGAACCGCATCGA
GCTGAAGGGCATCGACTTCAAGGAGGACGGCAACATCCTGGGGCACAAGC
TGGAGTACAACTACAACAGCCACAACGTCTATATCATGGCCGACAAGCAG
AAGAACGGCATCAAGGTGAACTTCAAGATCCGCCACAACATCGAGGACGG
CAGCGTGCAGCTCGCCGACCACTACCAGCAGAACACCCCCATCGGCGACG
GCCCCGTGCTGCTGCCCGACAACCACTACCTGAGCACCCAGTCCGCCCTG
AGCAAAGACCCCAACGAGAAGCGCGATCACATGGTCCTGCTGGAGTTCGT
GACCGCCGCCGGGATCACTCTCGGCATGGACGAGCTGTACAAGTAGGTCG
ACGTTTATTTGTGAAATTTGTGATGCTATTGCTTTATTTGTAACCATCTA
GCTTTATTTGTGAAATTTGTGATGCTATTGCTTTATTTGTAACCATTATA
AGCTGCAATAAACAAGTTAACAACAACAATTGCATTCATTTTATGTTTCA
GGTTCAGGGGGAGATGTGGGAGGTTTTTTAAAGTTTAAACAGGAACCCCT
AGTGATGGAGTTGGCCACTCCCTCTCTGCGCGCTCGCTCGCTCACTGAGG
CCGGGCGACCAAAGGTCGCCCGACGCCCGGGCGGCCTCAGTGAGCGAGCG
AGCGCGCAGCTGCCTGCAGGACATGTGAGCAAAAGGCCAGCAAAAGGCCA
GGAACCGTAAAAAGGCCGCGTTGCTGGCGTTTTTCCATAGGCTCCGCCCC
CCTGACGAGCATCACAAAAATCGACGCTCGCGGCCGCTCTAGAGATCATG
GAGATAATTAAAATGATAACCATCTCGCAAATAAATAAGTATTTTACTGT
TTTCGTAACAGTTTTGTAATAAAAAAACCTATAAATATTCCGGATTATTC
ATACCGTCCCACCATCGGGCGCGGATCCGCCGCCCTGGCGGGGTTTTACG
AGATTGTGATTAAGGTCCCCAGCGACCTTGACGAGCATCTGCCCGGCATT
TCTGACAGCTTTGTGAACTGGGTGGCCGAGAAGGAGTGGGAGTTGCCGCC
AGATTCTGACTTGGATCTGAATCTGATTGAGCAGGCACCCCTGACCGTGG
CCGAGAAGCTGCAGCGCGACTTTCTGACGGAGTGGCGCCGTGTGAGTAAG
GCCCCGGAGGCCCTTTTCTTTGTGCAATTTGAGAAGGGAGAGAGCTACTT
CCACTTACACGTGCTCGTGGAAACCACCGGGGTGAAATCCTTAGTTTTGG
GACGTTTCCTGAGTCAGATTCGCGAAAAACTGATTCAGAGAATTTACCGC
GGGATCGAGCCGACTTTGCCAAACTGGTTCGCGGTCACAAAGACCAGAAA
CGGCGCCGGAGGCGGGAACAAGGTGGTGGACGAGTGCTACATCCCCAATT
ACTTGCTCCCCAAAACCCAGCCTGAGCTCCAGTGGGCGTGGACTAATTTA
GAACAGTATTTAAGCGCCTGTTTGAATCTCACGGAGCGTAAACGGTTGGT
GGCGCAGCATCTGACGCACGTGTCGCAGACGCAGGAGCAGAACAAAGAGA
ATCAGAATCCCAATTCTGACGCGCCGGTGATCAGATCAAAAACTTCAGCC
AGGTACATGGAGCTGGTCGGGTGGCTCGTGGACAAGGGGATTACCTCGGA
GAAGCAGTGGATACAGGAGGACCAGGCCTCATACATCTCCTTCAATGCGG
CCTCCAACTCGCGGTCCCAAATCAAGGCTGCCTTGGACAATGCGGGAAAG
ATTATGAGCCTGACTAAAACCGCCCCCGACTACCTGGTGGGCCAGCAGCC
CGTGGAGGACATTTCCAGCAATCGGATTTATAAAATTTTGGAACTAAACG
GGTACGATCCCCAATATGCGGCTTCCGTCTTTCTGGGATGGGCCACGAAA
AAGTTCGGCAAGAGGAACACCATCTGGCTGTTTGGGCCTGCAACTACCGG
GAAGACCAACATCGCGGAGGCCATAGCCCACACTGTGCCCTTCTACGGGT
GCGTAAACTGGACCAATGAGAACTTTCCCTTCAACGACTGTGTGGACAAG
ATGGTGATCTGGTGGGAGGAGGGGAAGATGACCGCCAAGGTCGTGGAGTC
GGCCAAAGCCATTCTCGGAGGAAGCAAGGTGCGCGTGGACCAGAAATGCA
AGTCCTCGGCCCAGATAGACCCGACTCCCGTGATCGTCACCTCCAACACC
AACATGTGCGCCGTGATTGACGGGAACTCAACGACCTTCGAACACCAGCA
GCCGTTGCAAGACCGGATGTTCAAATTTGAACTCACCCGCCGTCTGGATC
ATGACTTTGGGAAGGTCACCAAGCAGGAAGTCAAAGACTTTTTCCGGTGG
GCAAAGGATCACGTGGTTGAGGTGGAGCATGAATTCTACGTCAAAAAGGG
TGGAGCCAAGAAAAGACCCGCCCCCAGTGACGCAGATATAAGTGAGCCCA
AACGGGTGCGCGAGTCAGTTGCGCAGCCATCGACGTCAGACGCGGAAGCA
TCGATCAACTACGCAGACAGGTACCAAAACAAATGTTCTCGTCACGTGGG
CATGAATCTGATGCTGTTTCCCTGCAGACAATGCGAGAGAATGAATCAGA
ATTCAAATATCTGCTTCACTCACGGACAGAAAGACTGTTTAGAGTGCTTT
CCCGTGTCAGAATCTCAACCCGTTTCTGTCGTCAAAAAGGCGTATCAGAA
ACTGTGCTACATTCATCATATCATGGGAAAGGTGCCAGACGCTTGCACTG
CCTGCGATCTGGTCAATGTGGATTTGGATGACTGCATCTTTGAACAATAA
TCTAGAGCCTGCAGTCTCGACAAGCTTGTCGAGAAGTACTAGAGGATCAT
AATCAGCCATACCACATTTGTAGAGGTTTTACTTGCTTTAAAAAACCTCC
CACACCTCCCCCTGAACCTGAAACATAAAATGAATGCAATTGTTGTTGTT
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTATCATGTCTGGATCTGATCACTGCTTGAG
CCTAGGAGATCCGAACCAGATAAGTGAAATCTAGTTCCAAACTATTTTGT
CATTTTTAATTTTCGTATTAGCTTACGACGCTACACCCAGTTCCCATCTA
TTTTGTCACTCTTCCCTAAATAATCCTTAAAAACTCCATTTCCACCCCTC
CCAGTTCCCAACTATTTTGTCCGCCCACAGCGGGGCATTTTTCTTCCTGT
TATGTTTTTAATCAAACATCCTGCCAACTCCATGTGACAAACCGTCATCT
TCGGCTACTTT

SEQ ID NO: 9:TTCTCTGTCACAGAATGAAAATTTTTCTGTCATCTCT
TCGTTATTAATGTTTGTAATTGACTGAATATCAACGCTTATTTGCAGCCT
GAATGGCGAATGGGACGCGCCCTGTAGCGGCGCATTAAGCGCGGCGGGTG
TGGTGGTTACGCGCAGCGTGACCGCTACACTTGCCAGCGCCCTAGCGCCC
GCTCCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGT
GGGCCATCGCCCTGATAGACGGTTTTTCGCCCTTTGACGTTGGAGTCCAC
GTTCTTTAATAGTGGACTCTTGTTCCAAACTGGAACAACACTCAACCCTA
TCTCGGTCTATTCTTTTGATTTATAAGGGATTTTGCCGATTTCGGCCTAT
TGGTTAAAAAATGAGCTGATTTAACAAAAATTTAACGCGAATTTTAACAA
AATATTAACGTTTACAATTTCAGGTGGCACTTTTCGGGGAAATGTGCGCG
GAACCCCTATTTGTTTATTTTTCTAAATACATTCAAATATGTATCCGCTC
ATGAGACAATAACCCTGATAAATGCTTCAATAATATTGAAAAAGGAAGAG
TATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGAT
GCTGAAGATCAGTTGGGTGCACGAGTGGGTTACATCGAACTGGATCTCAA
CAGCGGTAAGATCCTTGAGAGTTTTCGCCCCGAAGAACGTTTTCCAATGA
TGAGCACTTTTAAAGTTCTGCTATGTGGCGCGGTATTATCCCGTATTGAC
GCCGGGCAAGAGCAACTCGGTCGCCGCATACACTATTCTCAGAATGACTT
GGTTGAGTACTCACCAGTCACAGAAAAGCATCTTACGGATGGCATGACAG
TAAGAGAATTATGCAGTGCTGCCATAACCATGAGTGATAACACTGCGGCC
AACTTACTTCTGACAACGATCGGAGGACCGAAGGAGCTAACCGCTTTTTT
GCACAACATGGGGGATCATGTAACTCGCCTTGATCGTTGGGAACCGGAGC
TGAATGAAGCCATACCAAACGACGAGCGTGACACCACGATGCCTGTAGCA
ATGGCAACAACGTTGCGCAAACTATTAACTGGCGAACTACTTACTCTAGC
TTCCCGGCAACAATTAATAGACTGGATGGAGGCGGATAAAGTTGCAGGAC
CACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAA
CTATGGATGAACGAAATAGACAGATCGCTGAGATAGGTGCCTCACTGATT
AAGCATTGGTAACTGTCAGACCAAGTTTACTCATATATACTTTAGATTGA
TTTAAAACTTCATTTTTAATTTAAAAGGATCTAGGTGAAGATCCTTTTTG
ATAATCTCATGACCAAAATCCCTTAACGTGAGTTTTCGTTCCACTGAGCG
TCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGG
TTTGTTTGCCGGATCAAGAGCTACCAACTCTTTTTCCGAAGGTAACTGGC
TTCAGCAGAGCGCAGATACCAAATACTGTCCTTCTAGTGTAGCCGTAGTT
AGGCCACCACTTCAAGAACTCTGTAGCACCGCCTACATACCTCGCTCTGC
TAATCCTGTTACCAGTGGCTGCTGCCAGTGGCGATAAGTCGTGTCTTACC
GGGTTGGACTCAAGACGATAGTTACCGGATAAGGCGCAGCGGTCGGGCTG
AACGGGGGGTTCGTGCACACAGCCCAGCTTGGAGCGAACGACCTACACCG
AACTGAGATACCTACAGCGTGAGCATTGAGAAAGCGCCACGCTTCCCGAA
GGGAGAAAGGCGGACAGGTATCCGGTAAGCGGCAGGGTCGGAACAGGAGA
GCGCACGAGGGAGCTTCCAGGGGGAAACGCCTGGTATCTTTATAGTCCTG
TCGGGTTTCGCCACCTCTGACTTGAGCGTCGATTTTTGTGATGCTCGTCA
GGGGGGCGGAGCCTATGGAAAAACGCCAGCAACGCGGCCTTTTTACGGTT
CCTGGCCTTTTGCTGGCCTTTTGCTCACATGTTCTTTCCTGCGTTATCCC
CTGATTCTGTGGATAACCGTATTACCGCCTTTGAGTGAGCTGATACCGCT
CGCCGCAGCCGAACGACCGAGCGCAGCGAGTCAGTGAGCGAGGAAGCGGA
AGAGCGCCTGATGCGGTATTTTCTCCTTACGCATCTGTGCGGTATTTCAC
ACCGCAGACCAGCCGCGTAACCTGGCAAAATCGGTTACGGTTGAGTAATA
AATGGATGCCCTGCGTAAGCGGGTGTGGGCGGACAATAAAGTCTTAAACT
GAACAAAATAGATCTAAACTATGACAATAAAGTCTTAAACTAGACAGAAT
AGTTGTAAACTGAAATCAGTCCAGTTATGCTGTGAAAAAGCATACTGGAC
TTTTGTTATGGCTAAAGCAAACTCTTCATTTTCTGAAGTGCAAATTGCCC
GTCGTATTAAAGAGGGGCGTGGCCAAGGGCATGGTAAAGACTATATTCGC
GGCGTTGTGACAATTTACCGAACAACTCCGCGGCCGGGAAGCCGATCTCG
GCTTGAACGAATTGTTAGGTGGCGGTACTTGGGTCGATATCAAAGTGCAT
CACTTCTTCCCGTATGCCCAACTTTGTATAGAGAGCCACTGCGGGATCGT
CACCGTAATCTGCTTGCACGTAGATCACATAAGCACCAAGCGCGTTGGCC
TCATGCTTGAGGAGATTGATGAGCGCGGTGGCAATGCCCTGCCTCCGGTG
CTCGCCGGAGACTGCGAGATCATAGATATAGATCTCACTACGCGGCTGCT
CAAACCTGGGCAGAACGTAAGCCGCGAGAGCGCCAACAACCGCTTCTTGG
TCGAAGGCAGCAAGCGCGATGAATGTCTTACTACGGAGCAAGTTCCCGAG
GTAATCGGAGTCCGGCTGATGTTGGGAGTAGGTGGCTACGTCTCCGAACT
CACGACCGAAAAGATCAAGAGCAGCCCGCATGGATTTGACTTGGTCAGGG
CCGAGCCTACATGTGCGAATGATGCCCATACTTGAGCCACCTAACTTTGT
TTTAGGGCGACTGCCCTGCTGCGTAACATCGTTGCTGCTGCGTAACATCG
TTGCTGCTCCATAACATCAAACATCGACCCACGGCGTAACGCGCTTGCTG
CTTGGATGCCCGAGGCATAGACTGTACAAAAAAACAGTCATAACAAGCCA
TGAAAACCGCCACTGCGCCGTTACCACCGCTGCGTTCGGTCAAGGTTCTG
GACCAGTTGCGTGAGCGCATACGCTACTTGCATTACAGTTTACGAACCGA
ACAGGCTTATGTCAACTGGGTTCGTGCCTTCATCCGTTTCCACGGTGTGC
GTCACCCGGCAACCTTGGGCAGCAGCGAAGTCGAGGCATTTCTGTCCTGG
CTGGCGAACGAGCGCAAGGTTTCGGTCTCCACGCATCGTCAGGCATTGGC
GGCCTTGCTGTTCTTCTACGGCAAGGTGCTGTGCACGGATCTGCCCTGGC
TTCAGGAGATCGGTAGACCTCGGCCGTCGCGGCGCTTGCCGGTGGTGCTG
ACCCCGGATGAAGTGGTTCGCATCCTCGGTTTTCTGGAAGGCGAGCATCG
TTTGTTCGCCCAGGACTCTAGCTATAGTTCTAGTGGTTGGCCTACGTACC
CGTAGTGGCTATGGCAGGGCTTGCCGCCCCGACGTTGGCTGCGAGCCCTG
GGCCTTCACCCGAACTTGGGGGTTGGGGTGGGGAAAAGGAAGAAACGCGG
GCGTATTGGTCCCAATGGGGTCTCGGTGGGGTATCGACAGAGTGCCAGCC
CTGGGACCGAACCCCGCGTTTATGAACAAACGACCCAACACCCGTGCGTT
TTATTCTGTCTTTTTATTGCCGTCATAGCGCGGGTTCCTTCCGGTATTGT
CTCCTTCCGTGTTTCAGTTAGCCTCCCCCATCTCCCGGTACCGCATGCCT
TAGTTGAACTCGAACTTCTTGTACTTGCAGTTGAGCTTTTGCTCGGCGAA
TGTGATGGCGTCGGAGAGTGGCACCAGTCCCTGCAGGATCAAGGCCAAGA
GCTTCAGCAAGTTGTTGTGCAGTGTAGTTGACTCGCGACGGTTGACCTTG
CCGATCACGAACATGTGGTGCTTGAATCTGTTGTACTTTTGGATGACCTG
GCTCACATCAACGTCCTTGATTTCGCCAGAGATCCAGTAGAACTCCTTGT
TCTTCTTAGCGATGGTCAGCCTTTCCTCGTTCTTGAAGGACAACACGATG
AAGTTGTGACCCTTAACGTTATCGACGTTTTGAGTCAAGTACTGCTCAAC
GATGTGCATGCTTCCGTCTTCCTTCTTGACCTTCTTGAGGTTCTCGGCGT
TGTTGTTAGAAGCGATGTTGTCGTGGTACTTGTAGTTGTTGAAGAGCAGG
TTAGCGACGGACGAGTACTTGTATGTCAGAGTGCTCTTCTTGTTCACGAT
GAGGTTCAGGTTATCAACGACGTACTTGTTGGGAGGGTTGTCCGGGAATT
GGACGGACTCCGAGTACTTGAGGATCTGGGAAACGTATGGCGAGACGAAG
AAGTTGTTAGAGGCGGTTTCGATCTCGTTTGATTCCTTGCGGCTCAGCAT
GATGGGCAAAGTGAAGAGGTTCTTGTCCTGGTACATCTCGTACAACTTCG
ACAAGAGGAATCCACACTTACGCTCGCCGAGTGATTGCAGCAAGGTCACG
AAGGTTGTCTGAGCGTAGTACATATCGAGGTTGAAGTAGGAGGTCAGAGC
GGCCTTGAATGTGTGGTGGACGTCCACGAAGTGACACTTCTTGCAGTTTT
GAGCGGCGGTCTCATCGTTGCAGACGTCCTGTGAGTGTGGGATTTCGATG
CCAGTCTCCTTAACCAGGTTGTAGCTGATCATGAAGCGGATCTTATCGAA
GGTCACAACGAACACACGGTTGTCAACCATGTAGTAGTTGTTTGTGTACT
CGTAGACCACGTTAGAGACGTACTTGGCGAAGATGATTTCGAAAGGCTTC
ACCTCGGACTTCTTAACGACGAACATGTAGTAACCGGTTTCAGACATGTG
ATCGGAGAATCTGTTCGAGTTGTAGTCGTTATCGTCGAACCTCATCAGGT
AGGGGGCGAAGTCGTTTGTGAAGTAGTGAGTGATCTCCTGGGTGGAAGCC
ACTGTACAGATGTTAGTGTTGTGGTTGATGGTTTGTTCCAGTGTAGCGCA
TGACTGGATGGTGCTCTTCTTGTACTTAGGTCTCAACTTGATCTTGTTGA
ATTGACCCACAACTCCCTGGGAGTTATCCAGGTACTCGTCCAACTTCCTC
TTGGTGCCTGTGGCCGACGGCTGGTTGACACCAGCAGAGTGTTCGAAGGA
CTCGGCGTGGTAAGCTGAGCTAGGAGAGGGTTGTTCGACCACTGGCTGTT
CGAGTGACTCGCTGTAGTAGGCAGAGGACACAGCTTCCTCCAGGTTGTCA
GTGGTCTTGAGCAGACACTCCACCAAATCGTTGTCAGTGAGCGAGTTAAC
TGAGGCGAGGAAGTTGCTAGCGGCAGCGGTTTCAGAGTCGGAGATGACAG
TATCAGCTCCGTCCGGGGTTGGGTGGTTGTAGTAAGACAAGTAATCGTTA
GGTTGCTTGTCGCAGAACTCCGAGTATGAGTTGTCGAAGCTAGCACGAGA
GGGAGTGCTGGCAGAGGTGTAGGAAGCGTTGAAGTTGATTTGAGTCATGG
TGACCTGGTTGTTCACGATCTTATCGCCACCTGTGTCCACCTGCAGTTGC
TGGGCCTCAGCGCAGGCTGAAGTGGCCTCACAGGAGTAGGGGCTGGAAGC
ACAGTTGGAAGTCATGATGTTTTCTTGGACGTTCAGGACGTGGCTGGATG
TACGGATCATGCGCCCGATGGTGGGACGGTATGAATAATCCGGAATATTT
ATAGGTTTTTTTATTACAAAACTGTTACGAAAACAGTAAAATACTTATTT
ATTTGCGAGATGGTTATCATTTTAATTATCTCCATGATCGGATCTCCTAG
GCTCAAGCAGTGATCAGATCCAGACATGATAAGATACATTGATGAGTTTG
GACAAACCACAACTAGAATGCAGTGAAAAAAATGCTTTATTTGTGAAATT
TGTGATGCTATTGCTTTATTTGTAACCATTATAAGCTGCAATAAACAAGT
TAACAACAACAATTGCATTTGTCTAGTACTTCTCGACGGTACCGCATGCT
ATGCATCAGCTGCTAGCACCATGGCTCGAGTTACAGATTACGAGTCAGGT
ATCTGGTGCCAATGGGGCGAGGCTCTGAATACACGCCATTAGTGTCCACA
GTAAAGTCCACATTAACAGACTTGTTGTAGTTGGAAGTGTACTGAATTTC
GGGATTCCAGCGTTTGCTGTTTTCCTTCTGCAGCTCCCACTCGATCTCCA
CGCTGACCTGTCCCGTGGAGTACTGTGTGATGAAGGAAGCAAACTTTGCC
GCACTGAAGGTGGTCGAAGGATTCGCAGGTACCGGGGTGTTCTTGATGAG
AATCTGTGGAGGAGGGTGTTTAAGTCCGAATCCACCCATGAGGGGAGAGG
GGTGAAAATGTCCGTCCGTGTGTGGAATCTTTGCCCAGATGGGCCCCTGA
AGGTACACATCTCTGTCCTGCCAGACCATGCCTGGAAGAACGCCTTGTGT
GTTGACATCTGCGGTAGCTGCTTGTCTGTTGCCTCTCTGGAGGTTGGTAG
ATACAGAACCATACTGCTCCGTAGCCACGGGATTGGTTGTCCTGATTTCC
TCTTCGTCTGTAATCATGACCTTTTCAATGTCCACATTTGTTTTCTCTGA
GCCTTGCTTCCCAAAGATGAGAACCCCGCTCTGAGGAAAAAACTTTTCTT
CATCGTCCTTGTGGCTTGCCATCGCCGGGCCCGGATTCACCAGAGAGTCT
CTGCCATTGAGGTGGTACTTGGTAGCTCCAGTCCACGAGTATTCACTGTT
GTTGTTATCCGCAGATGTCTTTGATACTCGCTGCTGGCGGTAACAGGGTC
CAGGAAGCCAGTTCCTAGACTGGTCCCGAATGTCACTCGCTCCGGCCTGA
GAAAACTGAAGCCTTGACTGCGTGGTGGTTCCACTTGGAGTGTTTGTTCT
GCTCAAGTAATACAGGTACTGGTCGATGAGAGGATTCATGAGACGGTCCA
GACTCTGGCTGTGAGCGTAGCTGCTGTGGAAAGGAACGTCCTCAAAAGTG
TAGCTGAAGGTAAAGTTGTTTCCGGTACGCAGCATCTGAGAAGGAAAGTA
CTCCAGGCAGTAAAATGAAGAGCGTCCTACTGCCTGACTCCCGTTGTTCA
GGGTGAGGTATCCATACTGTGGCACCATGAAGACGTCTGCTGGGAACGGC
GGGAGGCATCCTTGATGCGCCGAGCCGAGGACGTACGGGAGCTGGTACTC
CGAGTCAGTAAACACCTGAACCGTGCTGGTAAGGTTATTGGCAATCGTCG
TCGTACCGTCATTCTGCGTGACCTCTTTGACTTGAATGTTAAAGAGCTTG
AAGTTGAGTCTCTTGGGTCGGAATCCCCAGTTGTTGTTGATGAGTCTTTG
CCAGTCACGTGGTGAAAAGTGGCAGTGGAATCTGTTGAAGTCAAAATACC
CCCAAGGGGTGCTGTAGCCAAAGTAGTGATTGTCGTTCGAGGCTCCTGAT
TGGCTGGAAATTTGTTTGTAGAGGTGGTTGTTGTAGGTGGGCAGGGCCCA
GGTTCGGGTGCTGGTGGTGATGACTCTGTCGCCCATCCATGTGGAATCGC
AATGCCAATTTCCCGAGGAATTACCCACTCCGTCGGCGCCCTCGTTATTG
TCTGCCATTGGTGCGCCACTGCCTGTAGCCATCGTATTAGTTCCCAGACC
AGAGGGGGCTGCTGGTGGCTGTCCGAGAGGCTGGGGGTCAGGTACTGAGT
CTGCGTCTCCAGTCTGACCAAAATTCAATCTTTTTCTTGCAGGCTGCTGG
CCCGCCTTTCCGGTTCCCGAGGAGGAGTCTGGCTCCACAGGAGAGTGCTC
TACCGGCCTCTTTTTTCCCGGAGCCGTCTTAACAGGTTCCTCAACCAGGC
CCAGAGGTTCAAGAACCCTCTTTTTCGCCTGGAAGACTGCTCGTCCGAGG
TTGCCCCCAAAAGACGTATCTTCTTTAAGGCGCTCCTGGAACTCCGCGTC
GGCGTGGTTGTACTTGAGGTAAGGGTTGTCTCCGCTGTCGAGCTGCCGGT
CGTAGGCTTTGTCGTGCTCGAGGGCAGCGGCGTCTGCCTCGTTCACCGGC
TCTCCCTTGTCCAGTCCGTTGAAGGGTCCGAGGTACTTGTACCCGGGAAG
CACAAGGCCCCTGCTGTCGTCCTTATGCCGCTCTGCGGGCTTTGGTGGTG
GTGGGCCGGGTTTGAGCTTCCACCACTGTCTTATTCCTTCAGAGAGAGTG
TCCTCGAGCCAATCGGGTAGATAACCGTCGGCAGCCGTGGCGGCTCAAGT
CTTCGTCGAGTGATTGTAAATAAAATGTAATTTACAGTATAGTATTTTAA
TTAATATACAAATGATTTGATAATAATTCTTATTTAACTATAATATATTG
TGTTGGGTTGAATTAAAGGTCCGTATCTCCAGACTGCAGTTTAAATTGTG
TAATTTATGTAGCTGTAATTTTTACCTTATTAATATTTTTTACGCTTTGC
ATTCGACGACTGAACTCCCAAATATATGTTTAACTCGTCTTGGTCGTTTG
AATTTTTGTTGCTGTGTTTCCTAATATTTTCCATCACCTTAAATATGTTA
TTGTAATCCTCAATGTTGAACTTGCAATTGGACACGGCATAGTTTTCCAT
AGTCGTGTAAAACATGGTATTGGCTGCATTGTAATACATCCGACTGAGCG
GGTACGGATCTATGTGTTTGAGCAGCCTGTTCAAAAACTCTGCATCGTCG
CAAAACGGAATTTGGTCACGATCCCGGTCCGAAGCGCGCGGAATTCAAAG
CGCTACGTCAGCGACGTCATCGATTATTGCTCCAATACTAGGGTTTTGTT
TTACTATCTGTTCTGCGGTCCACACATCTTCTTCGCTAGTCACGTACGCA
AACAACTCGGGCCGCAAAGGTGTTTTAAAACATATCAGATTAGAATCTTT
TATAACTTGGCCGCATTGTAAATAGTTGTGCCAACGCGCGGGAAACATTT
TACACGTGCTTTCAATCTTATCTCATAAACGCAACTAAATATGATCCTTC
AACTCGCTTTACGAGTAGAATTCTACTTGTAACGCACGATTAATTATGAC
TCATAAGCTGATGTCATGTTTTGCACACGGCTCATAACCGAACTCGCTTT
ACGAGTAGAATTCTACGCGTAAAACACAATCAAGTATGAGTCATAAGCTG
ATGTCATGTTTTGCACACGGCTCATAACCGAACTGGCTTTACGAGTAGAA
TTCTACTTGTAACGCACGATCAGTGGATGATGTCATAAACTTATCTCATG
TTTTGCACACGGCTCATAACTTGAGTCATAAGCTGATATCAGAAGATTAT
TAACGTGTTTAGGTATGACTCATTTGTTTTCAAAACTGAACTCGCTTTAC
GGATAGAATTCTACTTGTAAAACACAATCAGGGGATGATGTCATTATACA
AATGATGTCATTTGTTTTTCAAAACTAAACTCGCTTTACGGGTAGAATTC
TACTTGTAAAACACAATCGAGGGATAATGTCATTTGTTATTCTCAAATAT
GATGTCATTTAAATTTTTATTATGCAAGAATTGTATGTTGCTGTGTCATT
TATGAATGTTTTATCGACAACTTGTTTATTTGTTTCATTGACTTGTTGTT
GTTCGTGATTATGATTAATTATAAATCTTTTGTTGGGGTCAACATCGATG
TATACGCCGCTACAGGGCGCGTACTATGGTTGCTTTGACGTATGCGGTGT
GAAATACCGCACAGATGCGTAAGGAGAAAATACCGCATCAGGCGCCCCTG
CAGGCAGCTGCGCGCTCGCTCGCTCACTGAGGCCGCCCGGGCAAAGCCCG
GGCGTCGGGCGACCTTTGGTCGCCCGGCCTCAGTGAGCGAGCGAGCGCGC
AGAGAGGGAGTGGCCAACTCCATCACTAGGGGTTCCTACGCGTTAGTTAT
TAATAGTAATCAATTACGGGGTCATTAGTTCATAGCCCATATATGGAGTT
CCGCGTTACATAACTTACGGTAAATGGCCCGCCTGGCTGACCGCCCAACG
ACCCCCGCCCATTGACGTCAATAATGACGTATGTTCCCATAGTAACGCCA
ATAGGGACTTTCCATTGACGTCAATGGGTGGAGTATTTACGGTAAACTGC
CCACTTGGCAGTACATCAAGTGTATCATATGCCAAGTACGCCCCCTATTG
ACGTCAATGACGGTAAATGGCCCGCCTGGCATTATGCCCAGTACATGACC
TTATGGGACTTTCCTACTTGGCAGTACATCTACGTATTAGTCATCGCTAT
TACCATGGTGATGCGGTTTTGGCAGTACATCAATGGGCGTGGATAGCGGT
TTGACTCACGGGGATTTCCAAGTCTCCACCCCATTGACGTCAATGGGAGT
TTGTTTTGGCACCAAAATCAACGGGACTTTCCAAAATGTCGTAACAACTC
CGCCCCATTGACGCAAATGGGCGGTAGGCGTGTACGGTGGGAGGTCTATA
TAAGCAGAGCTGGTTTAGTGAACCGTCAGATCCGCTAGCGCTACCGGTCG
CCACCATGGTGAGCAAGGGCGAGGAGCTGTTCACCGGGGTGGTGCCCATC
CTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCT
GCACCACCGGCAAGCTGCCCGTGCCCTGGCCCACCCTCGTGACCACCCTG
ACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTC
GAGGGCGACACCCTGGTGAACCGCATCGAGCTGAAGGGCATCGACTTCAA
GGAGGACGGCAACATCCTGGGGCACAAGCTGGAGTACAACTACAACAGCC
ACAACGTCTATATCATGGCCGACAAGCAGAAGAACGGCATCAAGGTGAAC
TTCAAGATCCGCCACAACATCGAGGACGGCAGCGTGCAGCTCGCCGACCA
CTACCAGCAGAACACCCCCATCGGCGACGGCCCCGTGCTGCTGCCCGACA
ACCACTACCTGAGCACCCAGTCCGCCCTGAGCAAAGACCCCAACGAGAAG
CGCGATCACATGGTCCTGCTGGAGTTCGTGACCGCCGCCGGGATCACTCT
CGGCATGGACGAGCTGTACAAGTAGGTCGACGTTTATTTGTGAAATTTGT
GATGCTATTGCTTTATTTGTAACCATCTAGCTTTATTTGTGAAATTTGTG
ATGCTATTGCTTTATTTGTAACCATTATAAGCTGCAATAAACAAGTTAAC
AACAACAATTGCATTCATTTTATGTTTCAGGTTCAGGGGGAGATGTGGGA
GGTTTTTTAAAGTTTAAACAGGAACCCCTAGTGATGGAGTTGGCCACTCC
CTCTCTGCGCGCTCGCTCGCTCACTGAGGCCGGGCGACCAAAGGTCGCCC
GACGCCCGGGCGGCCTCAGTGAGCGAGCGAGCGCGCAGCTGCCTGCAGGA
CATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAAT
CGACGCTCGCGGCCGCTCTAGAGATCATGGAGATAATTAAAATGATAACC
ATCTCGCAAATAAATAAGTATTTTACTGTTTTCGTAACAGTTTTGTAATA
AAAAAACCTATAAATATTCCGGATTATTCATACCGTCCCACCATCGGGCG
CGGATCCGCCGCCCTGGCGGGGTTTTACGAGATTGTGATTAAGGTCCCCA
GCGACCTTGACGAGCATCTGCCCGGCATTTCTGACAGCTTTGTGAACTGG
GTGGCCGAGAAGGAGTGGGAGTTGCCGCCAGATTCTGACTTGGATCTGAA
TCTGATTGAGCAGGCACCCCTGACCGTGGCCGAGAAGCTGCAGCGCGACT
TTCTGACGGAGTGGCGCCGTGTGAGTAAGGCCCCGGAGGCCCTTTTCTTT
GTGCAATTTGAGAAGGGAGAGAGCTACTTCCACTTACACGTGCTCGTGGA
AACCACCGGGGTGAAATCCTTAGTTTTGGGACGTTTCCTGAGTCAGATTC
GCGAAAAACTGATTCAGAGAATTTACCGCGGGATCGAGCCGACTTTGCCA
AACTGGTTCGCGGTCACAAAGACCAGAAACGGCGCCGGAGGCGGGAACAA
GGTGGTGGACGAGTGCTACATCCCCAATTACTTGCTCCCCAAAACCCAGC
CTGAGCTCCAGTGGGCGTGGACTAATTTAGAACAGTATTTAAGCGCCTGT
TTGAATCTCACGGAGCGTAAACGGTTGGTGGCGCAGCATCTGACGCACGT
GTCGCAGACGCAGGAGCAGAACAAAGAGAATCAGAATCCCAATTCTGACG
CGCCGGTGATCAGATCAAAAACTTCAGCCAGGTACATGGAGCTGGTCGGG
TGGCTCGTGGACAAGGGGATTACCTCGGAGAAGCAGTGGATACAGGAGGA
CCAGGCCTCATACATCTCCTTCAATGCGGCCTCCAACTCGCGGTCCCAAA
TCAAGGCTGCCTTGGACAATGCGGGAAAGATTATGAGCCTGACTAAAACC
GCCCCCGACTACCTGGTGGGCCAGCAGCCCGTGGAGGACATTTCCAGCAA
TCGGATTTATAAAATTTTGGAACTAAACGGGTACGATCCCCAATATGCGG
CTTCCGTCTTTCTGGGATGGGCCACGAAAAAGTTCGGCAAGAGGAACACC
ATCTGGCTGTTTGGGCCTGCAACTACCGGGAAGACCAACATCGCGGAGGC
CATAGCCCACACTGTGCCCTTCTACGGGTGCGTAAACTGGACCAATGAGA
ACTTTCCCTTCAACGACTGTGTGGACAAGATGGTGATCTGGTGGGAGGAG
GGGAAGATGACCGCCAAGGTCGTGGAGTCGGCCAAAGCCATTCTCGGAGG
AAGCAAGGTGCGCGTGGACCAGAAATGCAAGTCCTCGGCCCAGATAGACC
CGACTCCCGTGATCGTCACCTCCAACACCAACATGTGCGCCGTGATTGAC
GGGAACTCAACGACCTTCGAACACCAGCAGCCGTTGCAAGACCGGATGTT
CAAATTTGAACTCACCCGCCGTCTGGATCATGACTTTGGGAAGGTCACCA
AGCAGGAAGTCAAAGACTTTTTCCGGTGGGCAAAGGATCACGTGGTTGAG
GTGGAGCATGAATTCTACGTCAAAAAGGGTGGAGCCAAGAAAAGACCCGC
CCCCAGTGACGCAGATATAAGTGAGCCCAAACGGGTGCGCGAGTCAGTTG
CGCAGCCATCGACGTCAGACGCGGAAGCATCGATCAACTACGCAGACAGG
TACCAAAACAAATGTTCTCGTCACGTGGGCATGAATCTGATGCTGTTTCC
CTGCAGACAATGCGAGAGAATGAATCAGAATTCAAATATCTGCTTCACTC
ACGGACAGAAAGACTGTTTAGAGTGCTTTCCCGTGTCAGAATCTCAACCC
GTTTCTGTCGTCAAAAAGGCGTATCAGAAACTGTGCTACATTCATCATAT
CATGGGAAAGGTGCCAGACGCTTGCACTGCCTGCGATCTGGTCAATGTGG
ATTTGGATGACTGCATCTTTGAACAATAATCTAGAGCCTGCAGTCTCGAC
AAGCTTGTCGAGAAGTACTAGAGGATCATAATCAGCCATACCACATTTGT
AGAGGTTTTACTTGCTTTAAAAAACCTCCCACACCTCCCCCTGAACCTGA
AACATAAAATGAATGCAATTGTTGTTGTTAACTTGTTTATTGCAGCTTAT
AATGGTTACAAATAAAGCAATAGCATCACAAATTTCACAAATAAAGCATT
TTTTTCACTGCATTCTAGTTGTGGTTTGTCCAAACTCATCAATGTATCTT
ATCATGTCTGGATCTGATCACTGCTTGAGCCTAGGAGATCCGAACCAGAT
AAGTGAAATCTAGTTCCAAACTATTTTGTCATTTTTAATTTTCGTATTAG
CTTACGACGCTACACCCAGTTCCCATCTATTTTGTCACTCTTCCCTAAAT
AATCCTTAAAAACTCCATTTCCACCCCTCCCAGTTCCCAACTATTTTGTC
CGCCCACAGCGGGGCATTTTTCTTCCTGTTATGTTTTTAATCAAACATCC
TGCCAACTCCATGTGACAAACCGTCATCTTCGGCTACTTT

The two recombinant vectors were transformed into DH10Bac competent cells (Invitrogen) for blue-white screening, respectively. The white positive clones were picked and cultured, and the bacmids were extracted using PureLink™ HiPure Plasmid DNA Purification Kits (Invitrogen). The two types of bacmids were transfected into adherent Sf9 cells (Gibco), and the primary recombinant baculoviruses were harvested 3 days later and amplified to the P2 generation. Finally, the recombinant baculoviruses of P2 generation were named BV-Cap-ITR-Rep and BV-IE-hr1Cap-ITR-Rep, respectively, and the virus titers were determined by TCID50 method. For details of this part, refer to the Bac-to-Bac Expression System Operation Manual (Invitrogen).

### Example 2. Packaging and Titer Determination of rAAV2

The two types of recombinant baculoviruses of P2-generation were used to infect 25 mL of suspension Sf9 cells (density 3.0E+06 cell/mL) at MOI=0.1, and the cells were harvested after 4 days. 5 mL of TNT lysis solution (20 mM Tris-HCl, pH 7.5, 150 mM NaCl, 1% Triton X-100, 10 mmol/L MgCl₂) was used and incubated at room temperature for 1 h to lyse the cells. Add nuclease at a final concentration of 50 U/mL to digest the cell lysate at 37° C. for 2 h, take samples and use proteinase K (Tiangen) to digest the capsid at 56° C. for 1 h, and determine the titer by qPCR. The detection steps are as follows:

The standard used in the quantitative PCR is the pAAV-EGFP plasmid linearized by single restriction enzyme digestion with PvuI-HF (NEB) (the plasmid sequence is shown in SEQ ID NO: 22).

SEQ ID NO.22:ACATGTGAGCAAAAGGCCAGCAAAAGGCCAGGAACCG
TAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAA
GACACGACTTATCGCCACTGGCAGCAGCCACTGGTAACAGGATTAGCAGA
GCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAG
TTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAA
AAAAGGATCTCAAGAAGATCCTTTGATCTTTTCTACGGGGTCTGACGCTC
AGTGGAACGAAAACTCACGTTAAGGGATTTTGGTCATGAGATTATCAAAA
AGGATCTTCACCTAGATCCTTTTAAATTAAAAATGAAGTTTTAAATCAAT
CTAAAGTATATATGAGTAAACTTGGTCTGACAGTTACCAATGCTTAATCA
GTGAGGCACCTATCTCAGCGATCTGTCTATTTCGTTCATCCATAGTTGCC
TGACTCCCCGTCGTGTAGATAACTACGATACGGGAGGGCTTACCATCTGG
CCCCAGTGCTGCAATGATACCGCGAGACCCACGCTCACCGGCTCCAGATT
TATCAGCAATAAACCAGCCAGCCGGAAGGGCCGAGCGCAGAAGTGGTCCT
GCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCC
GGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCG
CAGTGTTATCACTCATGGTTATGGCAGCACTGCATAATTCTCTTACTGTC
ATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAA
TACGGGATAATACCGCGCCACATAGCAGAACTTTAAAAGTGCTCATCATT
GGAAAACGTTCTTCGGGGCGAAAACTCTCAAGGATCTTACCGCTGTTGAG
ATCCAGTTCGATGTAACCCACTCGTGCACCCAACTGATCTTCAGCATCTT
TTACTTTCACCAGCGTTTCTGGGTGAGCAAAAACAGGAAGGCAAAATGCC
GCAAAAAAGGGAATAAGGGCGACACGGAAATGTTGAATACTCATACTCTT
CCTTTTTCAATATTATTGAAGCATTTATCAGGGTTATTGTCTCATGAGCG
GATACATATTTGAATGTATTTAGAAAAATAAACAAATAGGGGTTCCGCGC
ACATTTCCCCGAAAAGTGCCACCTGACGTCTAAGAAACCATTATTATCAT
GACATTAACCTATAAAAATAGGCGTATCACGAGGCCCTTTCGTCTCGCGC
GTTTCGGTGATGACGGTGAAAACCTCTGACACATGCAGCTCCCGGAGACG
GTCACAGCTTGTCTGTAAGCGGATGCCGGGAGCAGACAAGCCCGTCAGGG
CGCGTCAGCGGGTGTTGGCGGGTGTCGGGGCTGGCTTAACTATGCGGCAT
CAGAGCAGATTGTACTGAGAGTGCACCATAAAATTGTAAACGTTAATATT
TTGTTAAAATTCGCGTTAAATTTTTGTTAAATCAGCTCATTTTTTAACCA
ATAGGCCGAAATCGGCAAAATCCCTTATAAATCAAAAGAATAGCCCGAGA
TAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCC
ACTACGTGAACCATCACCCAAATCAAGTTTTTTGGGGTCGAGGTGCCGTA
AAGCACTAAATCGGAACCCTAAAGGGAGCCCCCGATTTAGAGCTTGACGG
GGAAAGCCGGCGAACGTGGCGAGAAAGGAAGGGAAGAAAGCGAAAGGAGC
GGGCGCTAGGGCGCTGGCAAGTGTAGCGGTCACGCTGCGCGTAACCACCA
CACCCGCCGCGCTTAATGCGCCGCTACAGGGCGCGTACTATGGTTGCTTT
GACGTATGCGGTGTGAAATACCGCACAGATGCGTAAGGAGAAAATACCGC
ATCAGGCGCCCCTGCAGGCAGCTGCGCGCTCGCTCGCTCACTGAGGCCGC
CCGGGCAAAGCCCGGGCGTCGGGCGACCTTTGGTCGCCCGGCCTCAGTGA
GCGAGCGAGCGCGCAGAGAGGGAGTGGCCAACTCCATCACTAGGGGTTCC
TACGCGTTAGTTATTAATAGTAATCAATTACGGGGTCATTAGTTCATAGC
CCATATATGGAGTTCCGCGTTACATAACTTACGGTAAATGGCCCGCCTGG
CTGACCGCCCAACGACCCCCGCCCATTGACGTCAATAATGACGTATGTTC
CCATAGTAACGCCAATAGGGACTTTCCATTGACGTCAATGGGTGGAGTAT
TTACGGTAAACTGCCCACTTGGCAGTACATCAAGTGTATCATATGCCAAG
TACGCCCCCTATTGACGTCAATGACGGTAAATGGCCCGCCTGGCATTATG
CCCAGTACATGACCTTATGGGACTTTCCTACTTGGCAGTACATCTACGTA
TTAGTCATCGCTATTACCATGGTGATGCGGTTTTGGCAGTACATCAATGG
GCGTGGATAGCGGTTTGACTCACGGGGATTTCCAAGTCTCCACCCCATTG
ACGTCAATGGGAGTTTGTTTTGGCACCAAAATCAACGGGACTTTCCAAAA
TGTCGTAACAACTCCGCCCCATTGACGCAAATGGGCGGTAGGCGTGTACG
GTGGGAGGTCTATATAAGCAGAGCTGGTTTAGTGAACCGTCAGATCCGCT
AGCGCTACCGGTCGCCACCATGGTGAGCAAGGGCGAGGAGCTGTTCACCG
GGGTGGTGCCCATCCTGGTCGAGCTGGACGGCGACGTAAACGGCCACAAG
TTCAGCGTGTCCGGCGAGGGCGAGGGCGATGCCACCTACGGCAAGCTGAC
CCTGAAGTTCATCTGCACCACCGGCAAGCTGCCCGTGCCCTGGCCCACCC
TCGTGACCACCCTGACCTACGGCGTGCAGTGCTTCAGCCGCTACCCCGAC
CACATGAAGCAGCACGACTTCTTCAAGTCCGCCATGCCCGAAGGCTACGT
CCAGGAGCGCACCATCTTCTTCAAGGACGACGGCAACTACAAGACCCGCG
CCGAGGTGAAGTTCGAGGGCGACACCCTGGTGAACCGCATCGAGCTGAAG
GGCATCGACTTCAAGGAGGACGGCAACATCCTGGGGCACAAGCTGGAGTA
CAACTACAACAGCCACAACGTCTATATCATGGCCGACAAGCAGAAGAACG
GCATCAAGGTGAACTTCAAGATCCGCCACAACATCGAGGACGGCAGCGTG
CAGCTCGCCGACCACTACCAGCAGAACACCCCCATCGGCGACGGCCCCGT
GCTGCTGCCCGACAACCACTACCTGAGCACCCAGTCCGCCCTGAGCAAAG
ACCCCAACGAGAAGCGCGATCACATGGTCCTGCTGGAGTTCGTGACCGCC
GCCGGGATCACTCTCGGCATGGACGAGCTGTACAAGTAGGTCGACGTTTA
TTTGTGAAATTTGTGATGCTATTGCTTTATTTGTAACCATCTAGCTTTAT
TTGTGAAATTTGTGATGCTATTGCTTTATTTGTAACCATTATAAGCTGCA
ATAAACAAGTTAACAACAACAATTGCATTCATTTTATGTTTCAGGTTCAG
GGGGAGATGTGGGAGGTTTTTTAAAGTTTAAACAGGAACCCCTAGTGATG
GAGTTGGCCACTCCCTCTCTGCGCGCTCGCTCGCTCACTGAGGCCGGGCG
ACCAAAGGTCGCCCGACGCCCGGGCGGCCTCAGTGAGCGAGCGAGCGCGC
AGCTGCCTGCAGG

The sequences of the primers used in quantitative PCR are:
ITR Forward primer 5′-GGAACCCCTAGTGATGGAGTT-3′ (SEQ ID NO: 2)
ITR Reverse primer 5′-CGGCCTCAGTGAGCGA-3′ (SEQ ID NO: 3)

The running program of quantitative PCR was: 95° C. for 60 s, (95° C. for 15 s, 60° C. for 30 s, 40 cycles). After plotting the standard curve according to the Ct value obtained by quantitative PCR and the concentrations of the standard, the titer of the sample was calculated. The yields of rAAVs packaged using BV-Cap-ITR-Rep and BV-IE-hr1Cap-ITR-Rep were determined to be 2.39E+04 VG/cell (equivalent to 7.18 E+10 VG/mL) and 8.67 E+05 VG/cell (equivalent to 2.60 E+12 VG/mL), respectively.

### Example 3. Comparison of the Expression of EGFP in the Process of Cell Infection and the Expression of Cap Proteins in the Cell Lysates

After 48 h, 72 h, and 96 h of infection, the cells infected by the two baculoviruses were examined under an inverted fluorescence microscope. The optimized recombinant baculovirus can greatly reduce the expression of the target gene (the target gene in this example is EGFP) (FIG. 5). The reduction of the expression of the target gene (EGFP) seems to weaken the interference to rAAV packaging in cells, making it more conducive to the expression of rAAV packaging-related structural and functional proteins.

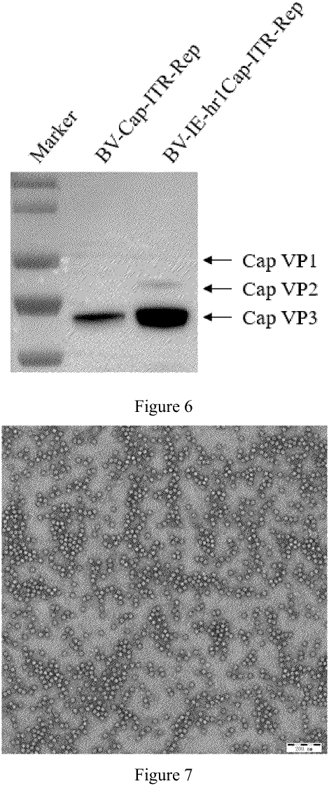

The same amount of cell lysates was assayed by Western blotting using Cap protein antibody (Progen) (FIG. 6). The results showed that after the optimized recombinant baculovirus infected the cells, the expression levels of Cap proteins in the cells were significantly increased.

### Example 4. Electron Microscopy Examination of Purified rAAV2 and Infectivity Determination

POROS™ CaptureSelect™ affinity chromatography media (Thermo) was used to purify rAAV2 from the cell lysate, and its titer was determined by fluorescent quantitative PCR (the determination method is the same as in Example 2). The purified rAAV2 was subject to negative staining treatment and examined using transmission electron microscopy (FIG. 7). The genome-packaged rAAV2 was solid particles, and the center of defective rAAV particles without nucleic acid was stained dark. Overall, the morphology of the purified rAAV2 is good, and the empty capsid rate is significantly reduced (~3%) compared with those obtained using traditional method (Benskey et al., 2016), suggesting that the optimized baculovirus vector construct significantly reduces the proportion of empty capsid viruses in the packaging product, which would significantly reduce the burden on downstream steps such as removal of empty capsid viruses.

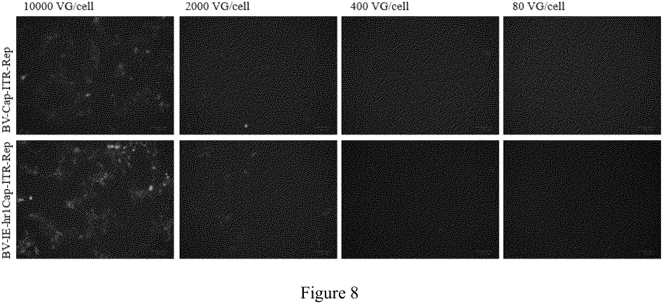

The purified rAAV2 was serially diluted and used to infect 293T cells cultured on 48-well plate (5.0 E+04 cells/well) at MOI=10000, 2000, 400, and 80, respectively. Two days after infection, the expression of EGFP was examined by fluorescence microscopy. The experimental results show that the rAAV2 prepared using this system has high in vitro infection activity (FIG. 8).

The above examples are intended to illustrate the disclosed embodiments of the present invention, and should not be construed as limiting the present invention. In addition, various modifications set forth herein, as well as changes in the method of the invention, will be obvious to those skilled in the art without departing from the scope and spirit of the invention. Although the invention has been specifically described in connection with various specific preferred embodiments of the invention, it should be understood that the invention should not be limited to these specific embodiments. In fact, various modifications as mentioned above which are obvious to those skilled in the art to obtain the invention should be included in the scope of the present invention.

1. A nucleic acid construct, wherein the nucleic acid construct comprises: AAV elements and a polynucleotide encoding an IE protein, and the AAV elements comprise a polynucleotide encoding Cap proteins, a polynucleotide encoding Rep proteins, and AAV cis-acting elements.

2. The nucleic acid construct according to claim 1, wherein the nucleic acid construct further comprises a polynucleotide encoding a recombination homologous region of baculovirus.

3. The nucleic acid construct according to claim 2, wherein the IE protein is encoded by one or more of Acie0, Acie01, and Acie02 genes, and/or, the recombination homologous region of baculovirus is selected from one or more of hr1, hr2, hr3, hr4, and hr5.

4. The nucleic acid construct according to claim 1, wherein the nucleic acid construct further comprises a promoter of an IE protein-coding gene, and the promoter of the IE protein-coding gene is selected from one or more of Gp64, pH, p6.9, and p10.

5. The nucleic acid construct according to claim 1, wherein the nucleic acid construct further comprises a baculovirus promoter connected to a recombination homologous region of baculovirus .

6. The nucleic acid construct according to claim 1, wherein the AAV cis-acting elements are selected from ITR sequences.

7. The nucleic acid construct according to claim 1, wherein the nucleic acid construct further comprises an exogenous gene of interest embedded in between AAV elements.

8. The nucleic acid construct according to claim 7, wherein the nucleic acid construct has a structure: IE gene expression cassette-Cap gene expression cassette-ITR-exogenous gene of interest expression cassette-ITR- Rep gene expression cassette.

9. The nucleic acid construct according to claim 1, wherein the nucleotide sequence of the nucleic acid construct is as shown in SEQ ID NO.1.

10. The nucleic acid construct according to claim 1, wherein the nucleic acid construct is an adeno-associated virus vector or a recombinant baculovirus vector.

11. A recombinant baculovirus, wherein the recombinant baculovirus is obtained by constructing the nucleic acid construct of claim 1 through a baculovirus system, or by jointly constructing the nucleic acid constructs containing any element in the nucleic acid construct of claim 1 through the baculovirus system.

12. An adeno-associated virus, wherein the adeno-associated virus is obtained by infecting cells with the recombinant baculovirus of claim 11 and packaging.

13. A cell line, wherein the cell line is a cell line infected with the recombinant baculovirus of claim 11.

14. An adeno-associated virus vector system, wherein the adeno-associated virus vector system comprises a baculovirus system and the nucleic acid construct of claim 1.

15. A method for constructing the nucleic acid construct of claim 1, wherein the method comprises integrating AAV elements carrying an exogenous gene of interest, a polynucleotide encoding an IE protein, and a polynucleotide encoding a recombination homologous region of baculovirus into a backbone of a baculovirus vector.

16. The method according to claim 15, wherein the method includes one or more of:
1) the AAV elements include a polynucleotide encoding Cap proteins, a polynucleotide encoding Rep proteins, and AAV cis-acting elements ;
2) The polynucleotide encoding the IE protein is selected from one or more of Acie0, Acie01, and Acie02 genes;
3) The backbone of the baculovirus vector is selected from one of pFastBacdual, pFastBac1, pFastBacHTA, pFastBacHTB, and pFastBacHTC;
4) The recombination homologous region of baculovirus is selected from one or more of hr1, hr2, hr3, hr4, and hr5.

17. A method for producing an adeno-associated virus, wherein the method comprises the step of infecting an insect cell line with the recombinant baculovirus of claim 11.

18. The nucleic acid construct according to claim 5, wherein the baculovirus promoter is one or more of pH, Gp64, p6.9, and p10.

19. The nucleic acid construct according to claim 10, wherein the recombinant baculovirus vector is a recombinant baculovirus shuttle vector.

20. The method according to claim 16, wherein the AAV cis-acting elements are ITR sequences.


SEQUENCE LISTING (22 sequences)

SEQ ID NO: 1 - DNA, 14057 bp, Artificial sequence
  Nucleotide sequence of nucleic acid construct
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgccttagttgaactcgaacttcttgtacttgcagttgagcttttgctcggcgaatgtgatggcgtcggagagtggcaccagtccctgcaggatcaaggccaagagcttcagcaagttgttgtgcagtgtagttgactcgcgacggttgaccttgccgatcacgaacatgtggtgcttgaatctgttgtacttttggatgacctggctcacatcaacgtccttgatttcgccagagatccagtagaactccttgttcttcttagcgatggtcagcctttcctcgttcttgaaggacaacacgatgaagttgtgacccttaacgttatcgacgttttgagtcaagtactgctcaacgatgtgcatgcttccgtcttccttcttgaccttcttgaggttctcggcgttgttgttagaagcgatgttgtcgtggtacttgtagttgttgaagagcaggttagcgacggacgagtacttgtatgtcagagtgctcttcttgttcacgatgaggttcaggttatcaacgacgtacttgttgggagggttgtccgggaattggacggactccgagtacttgaggatctgggaaacgtatggcgagacgaagaagttgttagaggcggtttcgatctcgtttgattccttgcggctcagcatgatgggcaaagtgaagaggttcttgtcctggtacatctcgtacaacttcgacaagaggaatccacacttacgctcgccgagtgattgcagcaaggtcacgaaggttgtctgagcgtagtacatatcgaggttgaagtaggaggtcagagcggccttgaatgtgtggtggacgtccacgaagtgacacttcttgcagttttgagcggcggtctcatcgttgcagacgtcctgtgagtgtgggatttcgatgccagtctccttaaccaggttgtagctgatcatgaagcggatcttatcgaaggtcacaacgaacacacggttgtcaaccatgtagtagttgtttgtgtactcgtagaccacgttagagacgtacttggcgaagatgatttcgaaaggcttcacctcggacttcttaacgacgaacatgtagtaaccggtttcagacatgtgatcggagaatctgttcgagttgtagtcgttatcgtcgaacctcatcaggtagggggcgaagtcgtttgtgaagtagtgagtgatctcctgggtggaagccactgtacagatgttagtgttgtggttgatggtttgttccagtgtagcgcatgactggatggtgctcttcttgtacttaggtctcaacttgatcttgttgaattgacccacaactccctgggagttatccaggtactcgtccaacttcctcttggtgcctgtggccgacggctggttgacaccagcagagtgttcgaaggactcggcgtggtaagctgagctaggagagggttgttcgaccactggctgttcgagtgactcgctgtagtaggcagaggacacagcttcctccaggttgtcagtggtcttgagcagacactccaccaaatcgttgtcagtgagcgagttaactgaggcgaggaagttgctagcggcagcggtttcagagtcggagatgacagtatcagctccgtccggggttgggtggttgtagtaagacaagtaatcgttaggttgcttgtcgcagaactccgagtatgagttgtcgaagctagcacgagagggagtgctggcagaggtgtaggaagcgttgaagttgatttgagtcatggtgacctggttgttcacgatcttatcgccacctgtgtccacctgcagttgctgggcctcagcgcaggctgaagtggcctcacaggagtaggggctggaagcacagttggaagtcatgatgttttcttggacgttcaggacgtggctggatgtacggatcatgcgcccgatggtgggacggtatgaataatccggaatatttataggtttttttattacaaaactgttacgaaaacagtaaaatacttatttatttgcgagatggttatcattttaattatctccatgatcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcatttgtctagtacttctcgacggtaccgcatgctatgcatcagctgctagcaccatggctcgagttacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgacctgtcccgtggagtactgtgtgatgaaggaagcaaactttgccgcactgaaggtggtcgaaggattcgcaggtaccggggtgttcttgatgagaatctgtggaggagggtgtttaagtccgaatccacccatgaggggagaggggtgaaaatgtccgtccgtgtgtggaatctttgcccagatgggcccctgaaggtacacatctctgtcctgccagaccatgcctggaagaacgccttgtgtgttgacatctgcggtagctgcttgtctgttgcctctctggaggttggtagatacagaaccatactgctccgtagccacgggattggttgtcctgatttcctcttcgtctgtaatcatgaccttttcaatgtccacatttgttttctctgagccttgcttcccaaagatgagaaccccgctctgaggaaaaaacttttcttcatcgtccttgtggcttgccatcgccgggcccggattcaccagagagtctctgccattgaggtggtacttggtagctccagtccacgagtattcactgttgttgttatccgcagatgtctttgatactcgctgctggcggtaacagggtccaggaagccagttcctagactggtcccgaatgtcactcgctccggcctgagaaaactgaagccttgactgcgtggtggttccacttggagtgtttgttctgctcaagtaatacaggtactggtcgatgagaggattcatgagacggtccagactctggctgtgagcgtagctgctgtggaaaggaacgtcctcaaaagtgtagctgaaggtaaagttgtttccggtacgcagcatctgagaaggaaagtactccaggcagtaaaatgaagagcgtcctactgcctgactcccgttgttcagggtgaggtatccatactgtggcaccatgaagacgtctgctgggaacggcgggaggcatccttgatgcgccgagccgaggacgtacgggagctggtactccgagtcagtaaacacctgaaccgtgctggtaaggttattggcaatcgtcgtcgtaccgtcattctgcgtgacctctttgacttgaatgttaaagagcttgaagttgagtctcttgggtcggaatccccagttgttgttgatgagtctttgccagtcacgtggtgaaaagtggcagtggaatctgttgaagtcaaaatacccccaaggggtgctgtagccaaagtagtgattgtcgttcgaggctcctgattggctggaaatttgtttgtagaggtggttgttgtaggtgggcagggcccaggttcgggtgctggtggtgatgactctgtcgcccatccatgtggaatcgcaatgccaatttcccgaggaattacccactccgtcggcgccctcgttattgtctgccattggtgcgccactgcctgtagccatcgtattagttcccagaccagagggggctgctggtggctgtccgagaggctgggggtcaggtactgagtctgcgtctccagtctgaccaaaattcaatctttttcttgcaggctgctggcccgcctttccggttcccgaggaggagtctggctccacaggagagtgctctaccggcctcttttttcccggagccgtcttaacaggttcctcaaccaggcccagaggttcaagaaccctctttttcgcctggaagactgctcgtccgaggttgcccccaaaagacgtatcttctttaaggcgctcctggaactccgcgtcggcgtggttgtacttgaggtaagggttgtctccgctgtcgagctgccggtcgtaggctttgtcgtgctcgagggcagcggcgtctgcctcgttcaccggctctcccttgtccagtccgttgaagggtccgaggtacttgtacccgggaagcacaaggcccctgctgtcgtccttatgccgctctgcgggctttggtggtggtgggccgggtttgagcttccaccactgtcttattccttcagagagagtgtcctcgagccaatcgggtagataaccgtcggcagccgtggcggctcaagtcttcgtcgagtgattgtaaataaaatgtaatttacagtatagtattttaattaatatacaaatgatttgataataattcttatttaactataatatattgtgttgggttgaattaaaggtccgtatctccagactgcagtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttggtcacgatcccggtccgaagcgcgcggaattcaaagcgctacgtcagcgacgtcatcgattattgctccaatactagggttttgttttactatctgttctgcggtccacacatcttcttcgctagtcacgtacgcaaacaactcgggccgcaaaggtgttttaaaacatatcagattagaatcttttataacttggccgcattgtaaatagttgtgccaacgcgcgggaaacattttacacgtgctttcaatcttatctcataaacgcaactaaatatgatccttcaactcgctttacgagtagaattctacttgtaacgcacgattaattatgactcataagctgatgtcatgttttgcacacggctcataaccgaactcgctttacgagtagaattctacgcgtaaaacacaatcaagtatgagtcataagctgatgtcatgttttgcacacggctcataaccgaactggctttacgagtagaattctacttgtaacgcacgatcagtggatgatgtcataaacttatctcatgttttgcacacggctcataacttgagtcataagctgatatcagaagattattaacgtgtttaggtatgactcatttgttttcaaaactgaactcgctttacggatagaattctacttgtaaaacacaatcaggggatgatgtcattatacaaatgatgtcatttgtttttcaaaactaaactcgctttacgggtagaattctacttgtaaaacacaatcgagggataatgtcatttgttattctcaaatatgatgtcatttaaatttttattatgcaagaattgtatgttgctgtgtcatttatgaatgttttatcgacaacttgtttatttgtttcattgacttgttgttgttcgtgattatgattaattataaatcttttgttggggtcaacatcgatgtatacgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccgtcgacgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagtttaaacaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcgcggccgctctagagatcatggagataattaaaatgataaccatctcgcaaataaataagtattttactgttttcgtaacagttttgtaataaaaaaacctataaatattccggattattcataccgtcccaccatcgggcgcggatccgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcatcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 2 - DNA, 21 bp, Artificial sequence
  ITR forward primer
ggaacccctagtgatggagtt

SEQ ID NO: 3 - DNA, 16 bp, Artificial sequence
  ITR reverse primer
cggcctcagtgagcga

SEQ ID NO: 4 - DNA, 2208 bp, Artificial sequence
  Cap gene sequence
acggctgccgacggttatctacccgattggctcgaggacactctctctgaaggaataagacagtggtggaagctcaaacccggcccaccaccaccaaagcccgcagagcggcataaggacgacagcaggggccttgtgcttcccgggtacaagtacctcggacccttcaacggactggacaagggagagccggtgaacgaggcagacgccgctgccctcgagcacgacaaagcctacgaccggcagctcgacagcggagacaacccttacctcaagtacaaccacgccgacgcggagttccaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggcgatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 5 - DNA, 1866 bp, Artificial sequence
  Rep gene sequence
ctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcatcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 6 - DNA, 1388 bp, Artificial sequence
  Nucleotide sequence of hr1-p6.9-p10 promoter
atcgatgttgaccccaacaaaagatttataattaatcataatcacgaacaacaacaagtcaatgaaacaaataaacaagttgtcgataaaacattcataaatgacacagcaacatacaattcttgcataataaaaatttaaatgacatcatatttgagaataacaaatgacattatccctcgattgtgttttacaagtagaattctacccgtaaagcgagtttagttttgaaaaacaaatgacatcatttgtataatgacatcatcccctgattgtgttttacaagtagaattctatccgtaaagcgagttcagttttgaaaacaaatgagtcatacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgttacaagtagaattctactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatgacgtcgctgacgtagcgctttgaattccgcgcgcttcggaccgggatcgtgaccaaattccgttttgcgacgatgcagagtttttgaacaggctgctcaaacacatagatccgtacccgctcagtcggatgtattacaatgcagccaataccatgttttacacgactatggaaaactatgccgtgtccaattgcaagttcaacattgaggattacaataacatatttaaggtgatggaaaatattaggaaacacagcaacaaaaattcaaacgaccaagacgagttaaacatatatttgggagttcagtcgtcgaatgcaaagcgtaaaaaatattaataaggtaaaaattacagctacataaattacacaatttaaactgcagtctggagatacggacctttaattcaacccaacacaatatattatagttaaataagaattattatcaaatcatttgtatattaattaaaatactatactgtaaattacattttatttacaatc

SEQ ID NO: 7 - DNA, 2202 bp, Artificial sequence
  Nucleotide sequence of pH-Acie01
aacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgatcatggagataattaaaatgataaccatctcgcaaataaataagtattttactgttttcgtaacagttttgtaataaaaaaacctataaatattccggattattcataccgtcccaccatcgggcgcatgatccgtacatccagccacgtcctgaacgtccaagaaaacatcatgacttccaactgtgcttccagcccctactcctgtgaggccacttcagcctgcgctgaggcccagcaactgcaggtggacacaggtggcgataagatcgtgaacaaccaggtcaccatgactcaaatcaacttcaacgcttcctacacctctgccagcactccctctcgtgctagcttcgacaactcatactcggagttctgcgacaagcaacctaacgattacttgtcttactacaaccacccaaccccggacggagctgatactgtcatctccgactctgaaaccgctgccgctagcaacttcctcgcctcagttaactcgctcactgacaacgatttggtggagtgtctgctcaagaccactgacaacctggaggaagctgtgtcctctgcctactacagcgagtcactcgaacagccagtggtcgaacaaccctctcctagctcagcttaccacgccgagtccttcgaacactctgctggtgtcaaccagccgtcggccacaggcaccaagaggaagttggacgagtacctggataactcccagggagttgtgggtcaattcaacaagatcaagttgagacctaagtacaagaagagcaccatccagtcatgcgctacactggaacaaaccatcaaccacaacactaacatctgtacagtggcttccacccaggagatcactcactacttcacaaacgacttcgccccctacctgatgaggttcgacgataacgactacaactcgaacagattctccgatcacatgtctgaaaccggttactacatgttcgtcgttaagaagtccgaggtgaagcctttcgaaatcatcttcgccaagtacgtctctaacgtggtctacgagtacacaaacaactactacatggttgacaaccgtgtgttcgttgtgaccttcgataagatccgcttcatgatcagctacaacctggttaaggagactggcatcgaaatcccacactcacaggacgtctgcaacgatgagaccgccgctcaaaactgcaagaagtgtcacttcgtggacgtccaccacacattcaaggccgctctgacctcctacttcaacctcgatatgtactacgctcagacaaccttcgtgaccttgctgcaatcactcggcgagcgtaagtgtggattcctcttgtcgaagttgtacgagatgtaccaggacaagaacctcttcactttgcccatcatgctgagccgcaaggaatcaaacgagatcgaaaccgcctctaacaacttcttcgtctcgccatacgtttcccagatcctcaagtactcggagtccgtccaattcccggacaaccctcccaacaagtacgtcgttgataacctgaacctcatcgtgaacaagaagagcactctgacatacaagtactcgtccgtcgctaacctgctcttcaacaactacaagtaccacgacaacatcgcttctaacaacaacgccgagaacctcaagaaggtcaagaaggaagacggaagcatgcacatcgttgagcagtacttgactcaaaacgtcgataacgttaagggtcacaacttcatcgtgttgtccttcaagaacgaggaaaggctgaccatcgctaagaagaacaaggagttctactggatctctggcgaaatcaaggacgttgatgtgagccaggtcatccaaaagtacaacagattcaagcaccacatgttcgtgatcggcaaggtcaaccgtcgcgagtcaactacactgcacaacaacttgctgaagctcttggccttgatcctgcagggactggtgccactctccgacgccatcacattcgccgagcaaaagctcaactgcaagtacaagaagttcgagttcaactaa

SEQ ID NO: 8 - DNA, 11248 bp, Artificial sequence
  Nucleotide sequence of pFBd-Cap-ITR-Rep
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgcttacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgacctgtcccgtggagtactgtgtgatgaaggaagcaaactttgccgcactgaaggtggtcgaaggattcgcaggtaccggggtgttcttgatgagaatctgtggaggagggtgtttaagtccgaatccacccatgaggggagaggggtgaaaatgtccgtccgtgtgtggaatctttgcccagatgggcccctgaaggtacacatctctgtcctgccagaccatgcctggaagaacgccttgtgtgttgacatctgcggtagctgcttgtctgttgcctctctggaggttggtagatacagaaccatactgctccgtagccacgggattggttgtcctgatttcctcttcgtctgtaatcatgaccttttcaatgtccacatttgttttctctgagccttgcttcccaaagatgagaaccccgctctgaggaaaaaacttttcttcatcgtccttgtggcttgccatcgccgggcccggattcaccagagagtctctgccattgaggtggtacttggtagctccagtccacgagtattcactgttgttgttatccgcagatgtctttgatactcgctgctggcggtaacagggtccaggaagccagttcctagactggtcccgaatgtcactcgctccggcctgagaaaactgaagccttgactgcgtggtggttccacttggagtgtttgttctgctcaagtaatacaggtactggtcgatgagaggattcatgagacggtccagactctggctgtgagcgtagctgctgtggaaaggaacgtcctcaaaagtgtagctgaaggtaaagttgtttccggtacgcagcatctgagaaggaaagtactccaggcagtaaaatgaagagcgtcctactgcctgactcccgttgttcagggtgaggtatccatactgtggcaccatgaagacgtctgctgggaacggcgggaggcatccttgatgcgccgagccgaggacgtacgggagctggtactccgagtcagtaaacacctgaaccgtgctggtaaggttattggcaatcgtcgtcgtaccgtcattctgcgtgacctctttgacttgaatgttaaagagcttgaagttgagtctcttgggtcggaatccccagttgttgttgatgagtctttgccagtcacgtggtgaaaagtggcagtggaatctgttgaagtcaaaatacccccaaggggtgctgtagccaaagtagtgattgtcgttcgaggctcctgattggctggaaatttgtttgtagaggtggttgttgtaggtgggcagggcccaggttcgggtgctggtggtgatgactctgtcgcccatccatgtggaatcgcaatgccaatttcccgaggaattacccactccgtcggcgccctcgttattgtctgccattggtgcgccactgcctgtagccatcgtattagttcccagaccagagggggctgctggtggctgtccgagaggctgggggtcaggtactgagtctgcgtctccagtctgaccaaaattcaatctttttcttgcaggctgctggcccgcctttccggttcccgaggaggagtctggctccacaggagagtgctctaccggcctcttttttcccggagccgtcttaacaggttcctcaaccaggcccagaggttcaagaaccctctttttcgcctggaagactgctcgtccgaggttgcccccaaaagacgtatcttctttaaggcgctcctggaactccgcgtcggcgtggttgtacttgaggtaagggttgtctccgctgtcgagctgccggtcgtaggctttgtcgtgctcgagggcagcggcgtctgcctcgttcaccggctctcccttgtccagtccgttgaagggtccgaggtacttgtacccgggaagcacaaggcccctgctgtcgtccttatgccgctctgcgggctttggtggtggtgggccgggtttgagcttccaccactgtcttattccttcagagagagtgtcctcgagccaatcgggtagataaccgtcggcagccgtggcggcgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatacgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggtcgacgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagtttaaacaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcgcggccgctctagagatcatggagataattaaaatgataaccatctcgcaaataaataagtattttactgttttcgtaacagttttgtaataaaaaaacctataaatattccggattattcataccgtcccaccatcgggcgcggatccgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcatcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 9 - DNA, 14777 bp, Artificial sequence
  Nucleotide sequence of pFBd-IE-hr1Cap-ITR-Rep
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgccttagttgaactcgaacttcttgtacttgcagttgagcttttgctcggcgaatgtgatggcgtcggagagtggcaccagtccctgcaggatcaaggccaagagcttcagcaagttgttgtgcagtgtagttgactcgcgacggttgaccttgccgatcacgaacatgtggtgcttgaatctgttgtacttttggatgacctggctcacatcaacgtccttgatttcgccagagatccagtagaactccttgttcttcttagcgatggtcagcctttcctcgttcttgaaggacaacacgatgaagttgtgacccttaacgttatcgacgttttgagtcaagtactgctcaacgatgtgcatgcttccgtcttccttcttgaccttcttgaggttctcggcgttgttgttagaagcgatgttgtcgtggtacttgtagttgttgaagagcaggttagcgacggacgagtacttgtatgtcagagtgctcttcttgttcacgatgaggttcaggttatcaacgacgtacttgttgggagggttgtccgggaattggacggactccgagtacttgaggatctgggaaacgtatggcgagacgaagaagttgttagaggcggtttcgatctcgtttgattccttgcggctcagcatgatgggcaaagtgaagaggttcttgtcctggtacatctcgtacaacttcgacaagaggaatccacacttacgctcgccgagtgattgcagcaaggtcacgaaggttgtctgagcgtagtacatatcgaggttgaagtaggaggtcagagcggccttgaatgtgtggtggacgtccacgaagtgacacttcttgcagttttgagcggcggtctcatcgttgcagacgtcctgtgagtgtgggatttcgatgccagtctccttaaccaggttgtagctgatcatgaagcggatcttatcgaaggtcacaacgaacacacggttgtcaaccatgtagtagttgtttgtgtactcgtagaccacgttagagacgtacttggcgaagatgatttcgaaaggcttcacctcggacttcttaacgacgaacatgtagtaaccggtttcagacatgtgatcggagaatctgttcgagttgtagtcgttatcgtcgaacctcatcaggtagggggcgaagtcgtttgtgaagtagtgagtgatctcctgggtggaagccactgtacagatgttagtgttgtggttgatggtttgttccagtgtagcgcatgactggatggtgctcttcttgtacttaggtctcaacttgatcttgttgaattgacccacaactccctgggagttatccaggtactcgtccaacttcctcttggtgcctgtggccgacggctggttgacaccagcagagtgttcgaaggactcggcgtggtaagctgagctaggagagggttgttcgaccactggctgttcgagtgactcgctgtagtaggcagaggacacagcttcctccaggttgtcagtggtcttgagcagacactccaccaaatcgttgtcagtgagcgagttaactgaggcgaggaagttgctagcggcagcggtttcagagtcggagatgacagtatcagctccgtccggggttgggtggttgtagtaagacaagtaatcgttaggttgcttgtcgcagaactccgagtatgagttgtcgaagctagcacgagagggagtgctggcagaggtgtaggaagcgttgaagttgatttgagtcatggtgacctggttgttcacgatcttatcgccacctgtgtccacctgcagttgctgggcctcagcgcaggctgaagtggcctcacaggagtaggggctggaagcacagttggaagtcatgatgttttcttggacgttcaggacgtggctggatgtacggatcatgcgcccgatggtgggacggtatgaataatccggaatatttataggtttttttattacaaaactgttacgaaaacagtaaaatacttatttatttgcgagatggttatcattttaattatctccatgatcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcatttgtctagtacttctcgacggtaccgcatgctatgcatcagctgctagcaccatggctcgagttacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgacctgtcccgtggagtactgtgtgatgaaggaagcaaactttgccgcactgaaggtggtcgaaggattcgcaggtaccggggtgttcttgatgagaatctgtggaggagggtgtttaagtccgaatccacccatgaggggagaggggtgaaaatgtccgtccgtgtgtggaatctttgcccagatgggcccctgaaggtacacatctctgtcctgccagaccatgcctggaagaacgccttgtgtgttgacatctgcggtagctgcttgtctgttgcctctctggaggttggtagatacagaaccatactgctccgtagccacgggattggttgtcctgatttcctcttcgtctgtaatcatgaccttttcaatgtccacatttgttttctctgagccttgcttcccaaagatgagaaccccgctctgaggaaaaaacttttcttcatcgtccttgtggcttgccatcgccgggcccggattcaccagagagtctctgccattgaggtggtacttggtagctccagtccacgagtattcactgttgttgttatccgcagatgtctttgatactcgctgctggcggtaacagggtccaggaagccagttcctagactggtcccgaatgtcactcgctccggcctgagaaaactgaagccttgactgcgtggtggttccacttggagtgtttgttctgctcaagtaatacaggtactggtcgatgagaggattcatgagacggtccagactctggctgtgagcgtagctgctgtggaaaggaacgtcctcaaaagtgtagctgaaggtaaagttgtttccggtacgcagcatctgagaaggaaagtactccaggcagtaaaatgaagagcgtcctactgcctgactcccgttgttcagggtgaggtatccatactgtggcaccatgaagacgtctgctgggaacggcgggaggcatccttgatgcgccgagccgaggacgtacgggagctggtactccgagtcagtaaacacctgaaccgtgctggtaaggttattggcaatcgtcgtcgtaccgtcattctgcgtgacctctttgacttgaatgttaaagagcttgaagttgagtctcttgggtcggaatccccagttgttgttgatgagtctttgccagtcacgtggtgaaaagtggcagtggaatctgttgaagtcaaaatacccccaaggggtgctgtagccaaagtagtgattgtcgttcgaggctcctgattggctggaaatttgtttgtagaggtggttgttgtaggtgggcagggcccaggttcgggtgctggtggtgatgactctgtcgcccatccatgtggaatcgcaatgccaatttcccgaggaattacccactccgtcggcgccctcgttattgtctgccattggtgcgccactgcctgtagccatcgtattagttcccagaccagagggggctgctggtggctgtccgagaggctgggggtcaggtactgagtctgcgtctccagtctgaccaaaattcaatctttttcttgcaggctgctggcccgcctttccggttcccgaggaggagtctggctccacaggagagtgctctaccggcctcttttttcccggagccgtcttaacaggttcctcaaccaggcccagaggttcaagaaccctctttttcgcctggaagactgctcgtccgaggttgcccccaaaagacgtatcttctttaaggcgctcctggaactccgcgtcggcgtggttgtacttgaggtaagggttgtctccgctgtcgagctgccggtcgtaggctttgtcgtgctcgagggcagcggcgtctgcctcgttcaccggctctcccttgtccagtccgttgaagggtccgaggtacttgtacccgggaagcacaaggcccctgctgtcgtccttatgccgctctgcgggctttggtggtggtgggccgggtttgagcttccaccactgtcttattccttcagagagagtgtcctcgagccaatcgggtagataaccgtcggcagccgtggcggctcaagtcttcgtcgagtgattgtaaataaaatgtaatttacagtatagtattttaattaatatacaaatgatttgataataattcttatttaactataatatattgtgttgggttgaattaaaggtccgtatctccagactgcagtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttggtcacgatcccggtccgaagcgcgcggaattcaaagcgctacgtcagcgacgtcatcgattattgctccaatactagggttttgttttactatctgttctgcggtccacacatcttcttcgctagtcacgtacgcaaacaactcgggccgcaaaggtgttttaaaacatatcagattagaatcttttataacttggccgcattgtaaatagttgtgccaacgcgcgggaaacattttacacgtgctttcaatcttatctcataaacgcaactaaatatgatccttcaactcgctttacgagtagaattctacttgtaacgcacgattaattatgactcataagctgatgtcatgttttgcacacggctcataaccgaactcgctttacgagtagaattctacgcgtaaaacacaatcaagtatgagtcataagctgatgtcatgttttgcacacggctcataaccgaactggctttacgagtagaattctacttgtaacgcacgatcagtggatgatgtcataaacttatctcatgttttgcacacggctcataacttgagtcataagctgatatcagaagattattaacgtgtttaggtatgactcatttgttttcaaaactgaactcgctttacggatagaattctacttgtaaaacacaatcaggggatgatgtcattatacaaatgatgtcatttgtttttcaaaactaaactcgctttacgggtagaattctacttgtaaaacacaatcgagggataatgtcatttgttattctcaaatatgatgtcatttaaatttttattatgcaagaattgtatgttgctgtgtcatttatgaatgttttatcgacaacttgtttatttgtttcattgacttgttgttgttcgtgattatgattaattataaatcttttgttggggtcaacatcgatgtatacgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggtcgacgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagtttaaacaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcgcggccgctctagagatcatggagataattaaaatgataaccatctcgcaaataaataagtattttactgttttcgtaacagttttgtaataaaaaaacctataaatattccggattattcataccgtcccaccatcgggcgcggatccgccgccctggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccaggtacatggagctggtcgggtggctcgtggacaaggggattacctcggagaagcagtggatacaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtggacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcatcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 10 - DNA, 786 bp, Unknown
  Nucleotide sequence of Acie0
atgataagaaccagcagtcacgtgctgaacgtccaggaaaatataatgacgtcaaactgtgcgtcatcgccatattcgtgcgaggcaacgtccgcttgcgcagaagctcagcaggtaatgatcgataactttgttttctttcacatgtacaacgccgacatacaaattgacgcaaagctgcaatgcggcgtgcgctcggccgcgtttgcaatgatcgacgataaacatttggaaatgtacaagcatagaatagagaataaatttttttattactatgatcaatgtgccgacattgccaaacccgaccgtctgcccgatgacgacggcgcgtgctgtcaccattttatttttgatgcccaacgtattattcaatgtattaaagagattgaaagcgcgtacggcgtgcgtgatcgcggcaatgtaatagtgttttatccgtacttgaaacagttgcgagacgcgttgaagctaattaaaaactcttttgcgtgttgttttaaaattataaattctatgcaaatgtacgtgaacgagttaatatcaaattgcctgttgtttattgaaaagctggaaactattaataaaactgttaaagttatgaatttgtttgtagacaatttggttttgtacgaatgcaatgtttgtaaagaaatatctacggatgaaagatttttaaagccaaaagaatgttgcgaatacgctatatgcaacgcgtgctgcgttaacatgtggaagacggccaccacgcacgcaaaatgtccagcgtgcaggacatcgtataaataa

SEQ ID NO: 11 - protein, 261 aa, Unknown
  Amino acid sequence of IE0 protein
MIRTSSHVLNVQENIMTSNCASSPYSCEATSACAEAQQVMIDNFVFFHMYNADIQIDAKLQCGVRSAAFAMIDDKHLEMYKHRIENKFFYYYDQCADIAKPDRLPDDDGACCHHFIFDAQRIIQCIKEIESAYGVRDRGNVIVFYPYLKQLRDALKLIKNSFACCFKIINSMQMYVNELISNCLLFIEKLETINKTVKVMNLFVDNLVLYECNVCKEISTDERFLKPKECCEYAICNACCVNMWKTATTHAKCPACRTSYK

SEQ ID NO: 12 - DNA, 1911 bp, Unknown
  Nucleotide sequence of Acie01
atgatccgtacatccagccacgtcctgaacgtccaagaaaacatcatgacttccaactgtgcttccagcccctactcctgtgaggccacttcagcctgcgctgaggcccagcaactgcaggtggacacaggtggcgataagatcgtgaacaaccaggtcaccatgactcaaatcaacttcaacgcttcctacacctctgccagcactccctctcgtgctagcttcgacaactcatactcggagttctgcgacaagcaacctaacgattacttgtcttactacaaccacccaaccccggacggagctgatactgtcatctccgactctgaaaccgctgccgctagcaacttcctcgcctcagttaactcgctcactgacaacgatttggtggagtgtctgctcaagaccactgacaacctggaggaagctgtgtcctctgcctactacagcgagtcactcgaacagccagtggtcgaacaaccctctcctagctcagcttaccacgccgagtccttcgaacactctgctggtgtcaaccagccgtcggccacaggcaccaagaggaagttggacgagtacctggataactcccagggagttgtgggtcaattcaacaagatcaagttgagacctaagtacaagaagagcaccatccagtcatgcgctacactggaacaaaccatcaaccacaacactaacatctgtacagtggcttccacccaggagatcactcactacttcacaaacgacttcgccccctacctgatgaggttcgacgataacgactacaactcgaacagattctccgatcacatgtctgaaaccggttactacatgttcgtcgttaagaagtccgaggtgaagcctttcgaaatcatcttcgccaagtacgtctctaacgtggtctacgagtacacaaacaactactacatggttgacaaccgtgtgttcgttgtgaccttcgataagatccgcttcatgatcagctacaacctggttaaggagactggcatcgaaatcccacactcacaggacgtctgcaacgatgagaccgccgctcaaaactgcaagaagtgtcacttcgtggacgtccaccacacattcaaggccgctctgacctcctacttcaacctcgatatgtactacgctcagacaaccttcgtgaccttgctgcaatcactcggcgagcgtaagtgtggattcctcttgtcgaagttgtacgagatgtaccaggacaagaacctcttcactttgcccatcatgctgagccgcaaggaatcaaacgagatcgaaaccgcctctaacaacttcttcgtctcgccatacgtttcccagatcctcaagtactcggagtccgtccaattcccggacaaccctcccaacaagtacgtcgttgataacctgaacctcatcgtgaacaagaagagcactctgacatacaagtactcgtccgtcgctaacctgctcttcaacaactacaagtaccacgacaacatcgcttctaacaacaacgccgagaacctcaagaaggtcaagaaggaagacggaagcatgcacatcgttgagcagtacttgactcaaaacgtcgataacgttaagggtcacaacttcatcgtgttgtccttcaagaacgaggaaaggctgaccatcgctaagaagaacaaggagttctactggatctctggcgaaatcaaggacgttgatgtgagccaggtcatccaaaagtacaacagattcaagcaccacatgttcgtgatcggcaaggtcaaccgtcgcgagtcaactacactgcacaacaacttgctgaagctcttggccttgatcctgcagggactggtgccactctccgacgccatcacattcgccgagcaaaagctcaactgcaagtacaagaagttcgagttcaactaa

SEQ ID NO: 13 - protein, 636 aa, Unknown
  Amino acid sequence of IE1 protein
MIRTSSHVLNVQENIMTSNCASSPYSCEATSACAEAQQLQVDTGGDKIVNNQVTMTQINFNASYTSASTPSRASFDNSYSEFCDKQPNDYLSYYNHPTPDGADTVISDSETAAASNFLASVNSLTDNDLVECLLKTTDNLEEAVSSAYYSESLEQPVVEQPSPSSAYHAESFEHSAGVNQPSATGTKRKLDEYLDNSQGVVGQFNKIKLRPKYKKSTIQSCATLEQTINHNTNICTVASTQEITHYFTNDFAPYLMRFDDNDYNSNRFSDHMSETGYYMFVVKKSEVKPFEIIFAKYVSNVVYEYTNNYYMVDNRVFVVTFDKIRFMISYNLVKETGIEIPHSQDVCNDETAAQNCKKCHFVDVHHTFKAALTSYFNLDMYYAQTTFVTLLQSLGERKCGFLLSKLYEMYQDKNLFTLPIMLSRKESNEIETASNNFFVSPYVSQILKYSESVQFPDNPPNKYVVDNLNLIVNKKSTLTYKYSSVANLLFNNYKYHDNIASNNNAENLKKVKKEDGSMHIVEQYLTQNVDNVKGHNFIVLSFKNEERLTIAKKNKEFYWISGEIKDVDVSQVIQKYNRFKHHMFVIGKVNRRESTTLHNNLLKLLALILQGLVPLSDAITFAEQKLNCKYKKFEFN

SEQ ID NO: 14 - DNA, 1227 bp, Unknown
  Nucleotide sequence of Acie02
atgagtcgccaaatcaacgccgccactcccagcagcagccgccgccacaggctgtctctcagccgtcgccgcatcaactttacaacatctcccgaagcccagccgtcttcaagcagtcgcagccagccgtcttcaagcagtcgcagccatcgccgtcaggagcggcgtcaggagcagcgtgtcagcgaagaaaacgtgcagattatcgggaacgtcaacgagccgttgacgcgcacctaccatcgtcagggtgtcacgtattacgtgcacggtcaggttaacattagcaatgacgatccgctattaagtcaagaggatgacgtcatactaattaatagtgaaaatgtggatcgtgaacggtttcccgacatcactgcccagcagtaccaggataacattgcgtcggagacagctgcgcagagggctctgcaacgaggtttagatcttgaggctcagctgatgaatgagattgccccaaggtctcccacttatagtccatcttattcgccgaattacgtaataccacagtcgccagatttgtttgcctcgccgcagtctccgcagccgcagcagcagcagcagcagcaatcagaacccgaagaagaagtagaggtttcgtgtaatatttgttttactacttttaaagacactaaaaacgtaaattcctcgtttgtgacttcgattcattgtaaccatgctgtgtgtttcaagtgttatgtcaagataattatggacaattctgtgtacaaatgtttttgcagcgctacttcatcagattgtcgcgtgtacaataagcacgggtatgtagaatttatgcccattaacgtcactcgtaaccaggattccatcaaacagcattggcgcgagcttttagaaaataacacggtcaacaatcacaccacggacttgaactatgtggagcaattgcaaaaagaactgtccgagctgcgagccaagaccagccaagttgaacataaaatgaccatgttaaacagcgactacattatgcttaaacacaagcatgctgtcgccgaattagatttacaaaaggcaaactatgacttgcaagaatctaccaagaaatcagaagagttgcaatcgactgtgaataatctgcaagaacaattgcgtaagcaggtggccgagtctcaagccaaattttcagagtttgagcgcagtaactctgatttagtttctaagttacaaactgttatgtctagacgttaa

SEQ ID NO: 15 - protein, 408 aa, Unknown
  Amino acid sequence of IE2 protein
MSRQINAATPSSSRRHRLSLSRRRINFTTSPEAQPSSSSRSQPSSSSRSHRRQERRQEQRVSEENVQIIGNVNEPLTRTYHRQGVTYYVHGQVNISNDDPLLSQEDDVILINSENVDRERFPDITAQQYQDNIASETAAQRALQRGLDLEAQLMNEIAPRSPTYSPSYSPNYVIPQSPDLFASPQSPQPQQQQQQQSEPEEEVEVSCNICFTTFKDTKNVNSSFVTSIHCNHAVCFKCYVKIIMDNSVYKCFCSATSSDCRVYNKHGYVEFMPINVTRNQDSIKQHWRELLENNTVNNHTTDLNYVEQLQKELSELRAKTSQVEHKMTMLNSDYIMLKHKHAVAELDLQKANYDLQESTKKSEELQSTVNNLQEQLRKQVAESQAKFSEFERSNSDLVSKLQTVMSRR

SEQ ID NO: 16 - DNA, 881 bp, Unknown
  Nucleotide sequence of hr1 of baculovirus
atcgatgttgaccccaacaaaagatttataattaatcataatcacgaacaacaacaagtcaatgaaacaaataaacaagttgtcgataaaacattcataaatgacacagcaacatacaattcttgcataataaaaatttaaatgacatcatatttgagaataacaaatgacattatccctcgattgtgttttacaagtagaattctacccgtaaagcgagtttagttttgaaaaacaaatgacatcatttgtataatgacatcatcccctgattgtgttttacaagtagaattctatccgtaaagcgagttcagttttgaaaacaaatgagtcatacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgttacaagtagaattctactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgat

SEQ ID NO: 17 - DNA, 990 bp, Unknown
  Nucleotide sequence of hr2 of baculovirus
tgagcaaaacacaaccggcaaattctcggcggccgtttgggaatgcggaataattgccatatgtaaatgatgtcatcggttctaactcgctttacgagtagaattctacgtgtaaaacataatcaagagatgatgtcatttgtttttcaaaactgaactcaagaaatgatgtcatttgtttttcaaaactgaactggctttacgagtagaattctacttgtaaaacacaatcgagagatgatgtcatattttgcacacggctctaattaaactcgctttacgagtaaaattctacttgtaacgcatgatcaagggatgatgtcattggatgagtcatttgtttttcaaaactaaactcgctttacgagtagaattctacttgtaaaacacaatcaagggatgatgtcattatacaaatgatgtcatttgtttttcaaaactaaactcgctttacgggtagaattctacttgtaaaacagcaactcgagggatgatgtcatcctttactcgatgattataaacgtgtttatgtatgactcatttgtttttcaaaactaaactcgctttacgagtagaattctacttgtaacgcacgatcaagggatgatgtcatttatttgtgcaaagctcgatgtcatcttttgcacacgattataaacacaatccaaataatgactcatttgttttcaaaactgaactcgctttacgagtagaattctacttgtaaaacacaatcaagggatgatgtcattttcaaaatgatgtcatttgtttttcaaaactaaactcgctttacgagtagaattctacttgtaaaacacaatcaagggatgatgtcattttaaaaatgatcatttgtttttcaaaactaaactcgctttacgagtagaattctacgtgtaaaacacaatcaagggatgatgtcatttactaaataaaataattatttaaataaaactgttttttattgtcaaatacacattgattcac

SEQ ID NO: 18 - DNA, 684 bp, Unknown
  Nucleotide sequence of hr3 of baculovirus
acgcgtagaattctacttgtaaagcaagttaaaataagccgtgtgcaaaaatgacatcagacaaatgacatcatctacctatcatgatcatgttaataatcatgttttaaaatgacatcagcttatgactaataattgatcgtgcgttacaagtagaattctactcgtaaagcgagtttagttttgaaaacaaatgagtcatcattaaacatgttaataatcgtgtataaaggatgacatcatccactaatcgtgcgttacaagtagaattctactcgtaaagcgagttcggttttgaaaaacaaatgacatcatttcttgattgtgttttacacgtagaattctactcgtaaagtatgttcagtttaaaaaacaaatgacatcattttacagatgacatcatttcttgattatgttttacaagtagaattctactcgtaaagcgagtttagttttaaaaaacaaatgacatcatctcttgattatgttttacaagtagaattctactcgtaaagcgagtttagttttgaaaaacaaatgacatcatctcttgattatgttttacaagtagaattctactcgtaaagcgagtttagttttgaaaaacaaatgacatcatcccttgatcatgcgttacaagtagaattctactcgtaaagcgagttgaattttgattacaatatt

SEQ ID NO: 19 - DNA, 675 bp, Unknown
  Nucleotide sequence of hr4 left of baculovirus
atgcatataattgtgtacaaaatatgactcattaatcgatcgtgcgttacaagtagaattctactggtaaagcaagttcggttgtgagccgtgtgcaaaacatgacatcataactaatcatgtttataatcatgtgcaaaatatgacatcatccgacgattgtgttttacaagtagaattctactcgtaaagcgagtttaaaaattttgtgacgtcaatgaaacaacgtgtaatattttttacaatatttaagtgaaacattatgacttccaataattttgtggatgtggatacgtttgcaagacaattgattacagataaatgtagtgctctaatcgaaagatgcggatctgttgccggcaaacattttagagattagtagagaaaggccagagacaagtattttgaggtgccaactcaaaaaaactatgaatacattaaaaaattatttttacgaacaaaatatatggacgattcgatagattataaagattttaacagacgcatcctattgatagtttttaaattcgctttaaacaagagcaccaactactttccatcgtactaaagagatcatcgaggtggccattaaacgtttaaacaaaattaaccccgatttaaagagttctccgcgcaatgcttcagcattacaaatgaatgtttggaaaatctaga

SEQ ID NO: 20 - DNA, 418 bp, Unknown
  Nucleotide sequence of hr4 right of baculovirus
aactggctttacgagtagaattctacttgtaaaacacaatcaagaaatgatgtcatttttgtacgtgattataaacatgtttaaacatggtacattgaacttaatttttgcaagttgataaactagattaatgtatgactcatttgtttgtgcaagttgataaacgtgattaatatatgactcatatgtttgtgcaaaaatggtgtcatcgtacaaactcgctttacgagtagaattctacttgtaaaacacaatcgagggatgatgtcatttgtagaatgatgtcatttgttttttcaaaaccgaactcgctttacgagtagaattctacttgtaaaacacaatcgagggatgatgtcatttgtagaatgatgtcatcgtacaaactcgctttacgagtagaattctagtaaaacac

SEQ ID NO: 21 - DNA, 725 bp, Unknown
  Nucleotide sequence of hr5 of baculovirus
ttgaaaatttattgcctaatattatttttgtcagttcgttgtcatttattaatttggatgatgtccatttgtttttaaaattgaactggctttacgagtagaattctacgcgtaaaacacaatcaagtatgagtcataagctgatgtcatgttttgcacacggctcataaccgaactggctttacgagtagaattctacttgtaacgcacgatcgagtggatgatggtcatttgtttttcaaatcgagatgatgtcatgttttgcacacgggctcataaactgctttacgagtagaattctacgtgtaacgcacgatcgattgatgagtcatttgttttgcaatatgatatcatacaatatgactcatttgtttttcaaaaccgaacttgatttacgggtagaattctactcgtaaagcacaatcaaaaagatgatgtcatttgtttttcaaaactgaactctcggctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaaagctatacgttaaatcaaacggacgttatggaattgtataatattaaatatgcaattgatccaacaaataaaattataatagagcaagtcgac

SEQ ID NO: 22 - DNA, 4400 bp, Artificial sequence
  pAAV-EGFP plasmid
acatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaggtcgacgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagtttaaacaggaacccctagtgatggagttggccactccctctctgcgcgctcgctcgctcactgaggccgggcgaccaaaggtcgcccgacgcccgggcggcctcagtgagcgagcgagcgcgcagctgcctgcagg